(19) United States
(12) Patent Application Publication
(10) Pub. No.: US 2023/0399287 A1
(43) Pub. Date: Dec. 14, 2023

(54) NOVEL COMPOUNDS

(71) Applicant: Sitryx Therapeutics Limited, Oxford (GB)

(72) Inventors: Matthew Colin Thor FYFE, Oxford (GB); Barry John TEOBALD, Oxford (GB)

(73) Assignee: Sitryx Therapeutics Limited, Oxford (GB)

(21) Appl. No.: 18/034,501

(22) PCT Filed: Oct. 29, 2021
(86) PCT No.: PCT/GB2021/052803

§ 371 Date: Apr. 28, 2023

(30) Foreign Application Priority Data
Oct. 29, 2020 (EP) 20204693.4
Nov. 4, 2020 (EP) 20205739.4
Mar. 8, 2021 (EP) 21161338.5
Aug. 17, 2021 (EP) 21191720.8

(51) Int. Cl. C07C 69/65; C07D 213/26; C07D 305/06; C07D 333/12; C07D 331/04; C07D 241/12; C07D 237/08; C07D 405/04; A61P 1/16

(52) U.S. Cl. CPC C07C 69/65; C07D 213/26; C07D 305/06; C07D 333/12; C07D 331/04; C07D 241/12; C07D 237/08; C07D 405/04; A61P 1/16

(57) ABSTRACT

The invention relates to compounds of formula (I) and to their use in treating or preventing an inflammatory disease or a disease associated with an undesirable immune response:

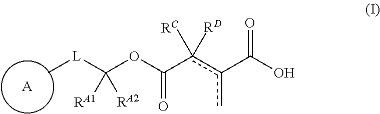

wherein A, L, R^{A1}, R^{A2}, R^{C} and R^{D} are as defined herein.

### CROSS-REFERENCE TO RELATED APPLICATIONS

This application is the National Stage of International Application No. PCT/GB2021/052803 filed Oct. 29, 2021, which claims priority to and benefit of European Application Nos. 20204693.4 filed Oct. 29, 2020, 20205739.4 filed Nov. 4, 2020, 21161338.5 filed Mar. 8, 2021 and 21191720.8 filed Aug. 17, 2021, each of which is herein incorporated by reference in its entirety.

### FIELD OF THE INVENTION

The present invention relates to compounds and their use in treating or preventing inflammatory diseases or diseases associated with an undesirable immune response, and to related compositions, methods and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (BrQck J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) preventing the association of PKCθ with the costimulatory receptor CD28 to reduce the production of IL-2 and block T-cell activation (Blewett M. M. et al., 2016); 6) reaction of the electrophilic α,β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1α (HIF-1α) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

Recently, it has been discovered that, during inflammatory macrophage activation, the CAC becomes anaplerotic and is diverted such that the unsaturated diacid itaconic acid, “itaconate”, is generated (Murphy M. P. and O'Neill L. A. J., 2018; O'Neill L. A. J. and Artyomov M. N., 2019; Yu X.-H. et al., 2019). Instead of being hydrated to isocitrate by aconitate hydratase, the CAC intermediate aconitate is decarboxylated by the protein product of immune-responsive gene 1 (IRG1), one of the most highly upregulated genes in macrophages under proinflammatory conditions, subsequently named aconitate decarboxylase 1, to produce itaconic acid (Michelucci A. et al., 2013). This unsaturated diacid is an inhibitor of the bacterial enzyme isocitrate lyase and, as such, it exerts anti-bacterial activity. In addition, itaconic acid has been shown to inhibit the CAC enzyme succinate dehydrogenase (SDH) (Ackermann et al., 1949), leading accordingly to succinate accumulation (Cordes T. et al., 2016). By inhibiting SDH, an enzyme critical for the inflammatory response (E. L. Mills et al., 2016), itaconate ameliorates inflammation in vitro and in vivo during macrophage activation and ischemia-reperfusion injury (Lampropoulou V. et al., 2016).

Like fumaric acid, itaconic acid is an α,β-unsaturated carboxylic acid. As such, it is a Michael acceptor which induces a global electrophilic stress response. In this regard, the itaconic acid diester dimethyl itaconate (DMI), like DMF, produces an anti-inflammatory response, reducing the expression levels of pro-inflammatory cytokines IL-1P, IL-6, IL-12 and IL-18 in lipopolysaccharide (LPS)-stimulated bone marrow-derived macrophages (WO2017/142855A1, incorporated herein by reference). This response appears to be mediated, in part, by NRF2 activation, via alkylation of KEAP1 cysteine residues by the electrophilic α,β-unsaturated ester moiety (Mills E. L. et al., 2018), which enhances the expression of downstream genes with anti-oxidant and anti-inflammatory capacities. Nevertheless, not all of the pronounced immunoregulatory effects engendered by DMI can be attributed to NRF2 activation. In particular, the modulation of Iκζ by DMI is independent of NRF2 and is mediated via upregulation of ATF3, a global negative regulator of immune activation that downregulates various cytokines, such as IL-6 (Bambouskova M. et al., 2018). Moreover, by inhibiting IKBζ protein production, DMI ameliorates IL-17-mediated pathologies, highlighting the therapeutic potential of this regulatory pathway (WO2019/036509A1, incorporated herein by reference). Further highlighting its pharmacologic potential, DMI has recently been reported to 1) demonstrate a protective effect on cerebral ischemia/reperfusion injury, thereby offering potential for the treatment of ischemic stroke (Zhang D. et al., 2019); 2) provide protection from the cardiotoxic effects of doxorubicin (Shan Q. et al., 2019); and 3) protect against lippolysacchride-induced mastitis in mice by activating MAPKs and NRFrf2 while inhibiting NF-κB signaling pathways (Zhao C. et al., 2019). Furthermore, DMI is said to have utility in preventing and treating ulcerative colitis and canceration thereof (CN110731955, Sun Yat-sen University Cancer Center); and has been reported to protect against fungal keratitis by activating the NRF2/HO-1 signalling pathway (Gu L. et al., 2020). Nevertheless, it should be noted that DMI is not metabolised to itaconic acid intracellularly (ElAzzouny M. et al., 2017). Other α,β-unsaturated esters exhibit IL-1β-lowering effects in macrophages by inhibiting the NLRP3 inflammasome (Cocco M. et al., 2017 and 2014), and have been demonstrated to inhibit the TLR4 pathway, leading ultimately to suppression of LPS-induced stimulation of NF-κB, tumour necrosis factor (TNF)-α, IL-1β and nitric oxide release (Zhang S. et al., 2012).

Other itaconic acid derivatives have been demonstrated to elicit anti-inflammatory effects (Bagavant G. et al., 1994). A notable example is 4-octyl itaconic acid (4OI), an itaconate derivative with improved cellular uptake. Since the α,β-unsaturated carboxylic acid is not esterified in 4OI, this electrophile exhibits low reactivity with biological thiols (Schmidt T. J. et al., 2007), much like the situation encountered with itaconic acid itself. As a result of its low reactivity/electrophilicity, the NRF2-activating effects of 4OI are not attenuated by GSH, in contrast to the findings with the much more reactive DMI. In this latter case, the α,β-unsaturated carboxylic acid is esterified and, as a consequence, the IL-6-lowering and NRF2-activating effects of DMI are reversed by the thiols N-acetylcysteine and GSH, respectively. Through the reaction with KEAP1 and the resulting NRF2 activation, as well as GAPDH inhibition (Liao S.-T. et al., 2019), 4OI has been demonstrated to produce a wide range of interesting biological effects, including: 1) protection of neuronal cells from hydrogen peroxide (Liu H. et al., 2018); 2) inhibition of proinflammatory cytokine production in peripheral blood mononuclear cells of SLE patients (Tang C. et al., 2018); and 3) protection of human umbilical vein endothelial cells from high glucose (Tang C. et al., 2019); 4) inhibition of osteoclastogenesis by suppressing the E3 ubiquitin ligase Hrd1 and activating NRF2 signaling (Sun X. et al., 2019); 5) induction of repression of STING by NRF2 and type I IFN production in cells from patients with STING-dependent interferonopathies (Olagnier D. et al., 2018); 6) protection against renal fibrosis via inhibiting the TGF-beta/Smad pathway, autophagy and reducing generation of reactive oxygen species (Tian F. et al., 2020); 7) reduction of brain viral burden in mice intracranially injected with Zika virus (Daniels B. P. et al. 2019); and 8) protection against liver ischemia-reperfusion injury (Yi F. et al. 2020). Furthermore, itaconate has been reported to modulate tricarboxylic acid and redox metabolism to mitigate reperfusion injury (Cordes T. et al., 2020). In addition, raised plasma itaconate levels demonstrate a clear correlation with reduction in rheumatoid arthritis disease activity scores following commencement of therapy with conventional disease modifying anti-rheumatic drug (cDMARD) therapy (Daly R. et al. 2019).

Artyomov et al. (WO2017/142855; WO2019/036509) disclose the use of itaconate, malonate or a derivative thereof as an immunomodulatory agent.

In spite of the above findings, there remains a need to identify and develop new itaconate derivatives possessing enhanced properties compared to currently marketed anti-inflammatory agents, such as DMF. The present inventors have now discovered, surprisingly, that certain itaconate monoesters are highly effective at reducing cytokine release in cells. These properties, amongst others, make them potentially more effective than DMI and/or dimethyl fumarate. Such compounds therefore appear to possess excellent anti-inflammatory properties.

### SUMMARY OF THE INVENTION

The present invention provides a compound of formula (I):

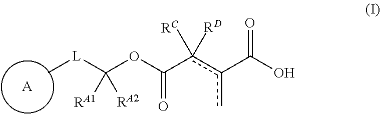

wherein,

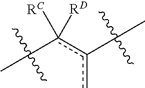

in the compound of formula (I) represents:

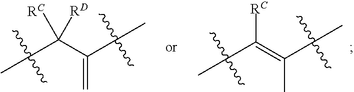

L is a bond, C₁₋₂alkylene or C≡C;
R^{A1} is H, C₁₋₄ alkyl, C₁₋₄ haloalkyl or C≡CH; and
R^{A2} is H, C₁₋₄ alkyl or C₁₋₄ haloalkyl; or
R^{A1} and R^{A2} join to form a C₃₋₆ cycloalkyl ring:

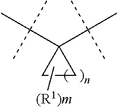

wherein:
R¹ is independently fluoro, methyl, cyano, OCH₃ or CF₃, or two R¹ groups which are attached to the same carbon atom join to form a C₃₋₄ cycloalkyl ring;
m is 0, 1 or 2;
n is 1, 2, 3 or 4; or
R^{A1} and R^{A2} join to form a 4-6 membered heterocyclic ring wherein the 4-6 membered heterocyclic ring is optionally substituted by one or more groups selected from oxo, fluoro or methyl;
A is phenyl, naphthyl or 5-6 membered heteroaryl wherein A is optionally substituted by one or more R², wherein A is other than pyrazolyl;
R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, halo, SF₅, SC₁₋₄ alkyl, SC₁₋₄ haloalkyl, —C≡C—C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring;
   wherein when A is phenyl, L is a bond or C≡C, and R² is R^{2a}, R^{2b}, R^{2c}, R^{2d} and R^{2e} the compound of formula (I) is:

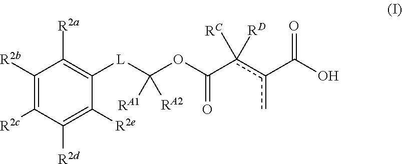

wherein:
   R^{2a} is H or halo;
   R^{2b} is H or halo;
   R^{2c} is C₁₋₄ alkyl, C₁₋₄ alkoxy, C₁₋₄ haloalkyl, C₁₋₄ haloalkoxy, cyano, Cl, Br, SF₅, SC₁₋₄ haloalkyl, —C≡C—C₁₋₂haloalkyl and phenyl, wherein the phenyl is optionally substituted by halo, or C₁₋₂ haloalkyl, and wherein the C₁₋₄ haloalkyl is optionally substituted by phenyl;
   R^{2d} is H or fluoro; and
   R^{2e} is H;
wherein when R^{2a}, R^{2d} and R^{2e} are H, R^{2b} is Cl and R^{2c} is CF₃, R^{A2} is other than H;
wherein when A is naphthyl, R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, chloro, bromo, SF₅, SC₁₋₄ alkyl, SC₁₋₄ haloalkyl, —C≡C—C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring; and
   wherein when A is thienyl, R² is C₁₋₄ haloalkyl;
R^{C} and R^{D} are independently selected from the group consisting of H, C₁₋₂ alkyl, hydroxy, methoxy and fluoro or, taken together, R^{C} and R^{D} combine to form a C₃₋₄ cycloalkyl ring;
   or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an undesirable immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

Compounds of formula (I)

Embodiments and preferences set out herein with respect to the compound of formula (I) apply equally to the pharmaceutical composition, compound for use, use and method aspects of the invention.

In one embodiment there is provided a compound of formula (I) as described above.

Suitably, there is provided a compound of formula (Ia):

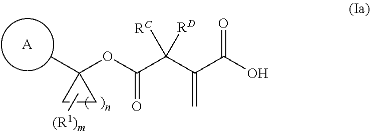

wherein,
R¹ is independently fluoro or methyl;
A is phenyl, naphthyl or 5-6 membered heteroaryl wherein A is optionally substituted by one or more R²
R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, halo, SF₅, SC₁₋₄ alkyl, and SC₁₋₄ haloalkyl; or, two R² groups are attached to adjacent carbon atoms and are joined to form a C₃₋₈ cycloalkyl or 4-7 membered heterocyclic ring;
R^{C} and R^{D} are independently selected from the group consisting of H, C₁₋₂ alkyl, hydroxy, methoxy and fluoro;
m is 0, 1 or 2;
n is 1, 2, 3 or 4;
   or a pharmaceutically acceptable salt and/or solvate thereof.

In another embodiment, there is provided a compound of formula (Ib):

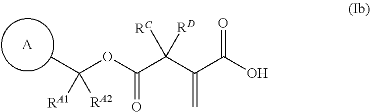

wherein,
R^{A1} is H, C₁₋₄ alkyl or C₁₋₄ haloalkyl; and
R^{A2} is C₁₋₄ alkyl or C₁₋₄ haloalkyl; or
R^{A1} and R^{A2} join to form a C₃₋₆ cycloalkyl ring:

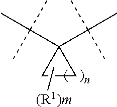

wherein:
R¹ is independently fluoro or methyl;
m is 0, 1 or 2;
n is 1, 2, 3 or 4;
A is phenyl, naphthyl or 5-6 membered heteroaryl wherein A is optionally substituted by one or more R²
R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, halo, SF₅, SC₁₋₄ alkyl, and SC₁₋₄ haloalkyl; or, two R² groups are attached to adjacent carbon atoms and are joined to form a C₃₋₈ cycloalkyl or 4-7 membered heterocyclic ring;
R^{C} and R^{D} are independently selected from the group consisting of H, C₁₋₂ alkyl, hydroxy, methoxy and fluoro;
   or a pharmaceutically acceptable salt and/or solvate thereof.

In another embodiment, there is provided a compound of formula (Ic):

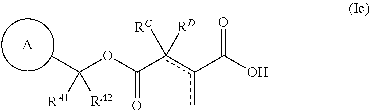

wherein,

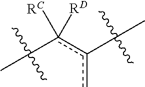

in the compound of formula (I) represents:

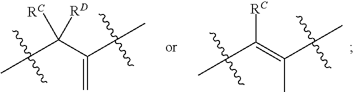

R^{A1} is H, C₁₋₄ alkyl, C₁₋₄ haloalkyl or C≡CH; and
R^{A2} is H, C₁₋₄ alkyl or C₁₋₄ haloalkyl; or
R^{A1} and R^{A2} join to form a C₃₋₆ cycloalkyl ring:

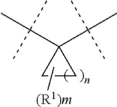

wherein:
R¹ is independently fluoro, methyl or cyano, or two R¹ groups which are attached to the same carbon atom join to form a C₃₋₄cycloalkyl ring;
m is 0, 1 or 2;
n is 1, 2, 3 or 4; or
R^{A1} and R^{A2} join to form a 4-6 membered heterocyclic ring;
A is phenyl, naphthyl or 5-6 membered heteroaryl wherein A is optionally substituted by one or two R²; wherein when A is phenyl, naphthyl or 6-membered heteroaryl, one R² is in the 4-position with respect to C(R^{A1})(R^{A2}) and wherein the R² group in the 4-position is other than fluoro;
R² is independently selected from the group consisting of C₁₋₄ alkyl, C₁₋₄ alkoxy, C₁₋₄ haloalkyl, wherein the C₁₋₄ haloalkyl is optionally substituted by phenyl, C₁₋₄ haloalkoxy, cyano, halo, SF₅, SC₁₋₄ haloalkyl, —C═C—C₁₋₂haloalkyl, and phenyl, wherein the phenyl is optionally substituted by halo or C₁₋₂ haloalkyl; or, two R² groups are attached to adjacent carbon atoms and are joined to form a C₃₋₈ cycloalkyl or 4-7 membered heterocyclic ring;
R^{C} and R^{D} are independently selected from the group consisting of H, C₁₋₂ alkyl, hydroxy, methoxy and fluoro or, taken together, R^{C} and R^{D} combine to form a C₃₋₄ cycloalkyl ring;
   or a pharmaceutically acceptable salt and/or solvate thereof.

In another embodiment, there is provided a compound of formula (If):

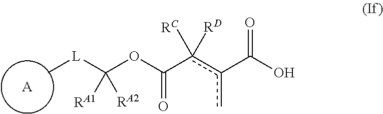

wherein,

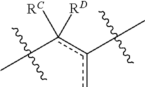

in the compound of formula (I) represents:

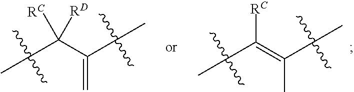

R^{A1} is H, C₁₋₄ alkyl, C₁₋₄ haloalkyl or C═CH; and
R^{A2} is H, C₁₋₄ alkyl or C₁₋₄ haloalkyl; or
R^{A1} and R^{A2} join to form a C₃₋₆ cycloalkyl ring:

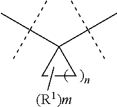

wherein:
R¹ is independently fluoro, methyl or cyano, or two R¹ groups which are attached to the same carbon atom join to form a C₃₋₄ cycloalkyl ring;
m is 0, 1 or 2;
n is 1, 2, 3 or 4; or
R^{A1} and R^{A2} join to form a 4-6 membered heterocyclic ring;
A is phenyl, naphthyl or 5-6 membered heteroaryl wherein A is optionally substituted by one or more R²
R² is independently selected from the group consisting of C₁₋₄ alkyl, C₁₋₄ alkoxy, C₁₋₄ haloalkyl, wherein the C₁₋₄ haloalkyl is optionally substituted by phenyl, C₁₋₄ haloalkoxy, hydroxy, CO₂H, cyano, methanesulfonyl, halo, SF₅, SC₁₋₄ alkyl, SC₁₋₄ haloalkyl, —C═C—C₁₋₂haloalkyl, and phenyl, wherein the phenyl is optionally substituted by halo or C₁₋₂ haloalkyl; or, two R² groups are attached to adjacent carbon atoms and are joined to form a C₃₋₈ cycloalkyl or 4-7 membered heterocyclic ring;
L is a bond, C₁₋₂alkylene or C≡C;
R^{C} and R^{D} are independently selected from the group consisting of H, C₁₋₂ alkyl, hydroxy, methoxy and fluoro or, taken together, R^{C} and R^{D} combine to form a C₃₋₄ cycloalkyl ring;
   or a pharmaceutically acceptable salt and/or solvate thereof.

In another embodiment there is provided a compound of formula (Ig):

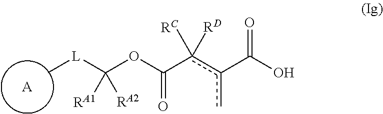

wherein,

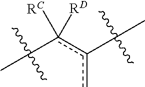

in the compound of formula (I) represents:

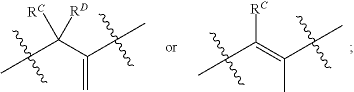

L is a bond, C₁₋₂alkylene or C≡C;
R^{A1} is H, C₁₋₄ alkyl, C₁₋₄ haloalkyl or C≡CH; and
R^{A2} is H, C₁₋₄ alkyl or C₁₋₄ haloalkyl; or
R^{A1} and R^{A2} join to form a C₃₋₆ cycloalkyl ring:

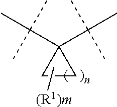

wherein:
R¹ is independently fluoro, methyl, cyano, OCH₃ or CF₃, or two R¹ groups which are attached to the same carbon atom join to form a C₃₋₄ cycloalkyl ring;
m is 0, 1 or 2;
n is 1, 2, 3 or 4; or
R^{A1} and R^{A2} join to form a 4-6 membered heterocyclic ring wherein the 4-6 membered heterocyclic ring is optionally substituted by one or more groups selected from oxo, fluoro or methyl;
A is phenyl, naphthyl or 5-6 membered heteroaryl wherein A is optionally substituted by one or more R², wherein A is other than pyrazinyl and pyrazolyl;
R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, halo, SF₅, SC₁₋₄ alkyl, SC₁₋₄ haloalkyl, —C≡C—C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring;
   wherein when A is phenyl, L is a bond or C≡C, and R² is R^{2a}, R^{2b}, R^{2c}, R^{2d} and R^{2e} the compound of formula (I) is:

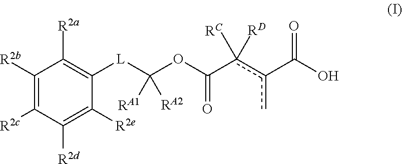

wherein:
   R^{2a} is H or halo;
   R^{2b} is H or halo;
   R^{2c} is C₁₋₄ alkyl, C₁₋₄ alkoxy, C₁₋₄ haloalkyl, C₁₋₄ haloalkoxy, cyano, Cl, Br, SF₅, SC₁₋₄ haloalkyl, —C≡C—C₁₋₂haloalkyl and phenyl, wherein the phenyl is optionally substituted by halo, or C₁₋₂ haloalkyl, and wherein the C₁₋₄ haloalkyl is optionally substituted by phenyl;
   R^{2d} is H or fluoro; and
   R^{2e} is H;
wherein when R^{2a}, R^{2b}, R^{2d} and R^{2e} are H, R²¹ is Cl and R², is CF₃, R^{A2} is other than H;
wherein when A is naphthyl, R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, chloro, bromo, SF₅, SC₁₋₄ alkyl, SC₁₋₄ haloalkyl, —C≡C—C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring; and
   wherein when A is thienyl, R² is C₁₋₄ haloalkyl;
R^{C} and R^{D} are independently selected from the group consisting of H, C₁₋₂ alkyl, hydroxy, methoxy and fluoro or, taken together, R^{C} and R^{D} combine to form a C₃₋₄ cycloalkyl ring;
   or a pharmaceutically acceptable salt and/or solvate thereof.

The references and embodiments for compounds of formula (I) apply to compounds of formula (Ia), (Ib), (Ic), (Id), (le), (If) and (Ig), where relevant.

The term “C₁₋₄ alkyl” (e.g. C₁₋₃ alkyl group, C₁₋₂ alkyl group or C₁ alkyl group) refers to a straight or branched fully saturated hydrocarbon group having from 1 to 4 carbon atoms. The term encompasses methyl, ethyl, n-propyl, isopropyl, n-butyl, isobutyl, sec-butyl and tert-butyl. The term “C₁₋₄ alkyl” also encompasses “C₁₋₄ alkylene” which is a bifunctional straight or branched fully saturated hydrocarbon group having from 1 to 4 carbon atoms. Example “C₁₋₄ alkylene” groups include methylene, ethylene, n-propylene and n-butylene.

The term “C₁₋₄ alkoxy” refers to a C₁₋₄ alkyl group (e.g. C₁₋₃ alkyl group, C₁₋₂ alkyl group or C1 alkyl group) as defined above, singularly bonded to oxygen. The term encompasses methoxy, ethoxy, 1-propoxy and 2-propoxy, and is suitably methoxy.

The term “C₁₋₄ haloalkyl” (e.g. C₁₋₃ haloalkyl group, C₁₋₂ haloalkyl group or C1 haloalkyl group) as used herein refers to a straight or a branched fully saturated hydrocarbon chain containing the specified number of carbon atoms and at least one halogen atom, such as fluoro or chloro, especially fluoro. An example of haloalkyl is CF₃. Further examples of haloalkyl are CHF₂ and CH₂CF₃.

The term “C₁₋₄ haloalkoxy” refers to a C₁₋₄ haloalkyl group (e.g. C₁₋₃ haloalkyl group, C₁₋₂ haloalkyl group or C, haloalkyl group) as defined above, singularly bonded to oxygen. Examples of C₁₋₄ haloalkoxy include OCF₃, OCHF₂ and OCH₂CF₃.

The term “C₃₋₈ cycloalkyl” (e.g. C₃₋₆ cycloalkyl, C₃₋₄ cycloalkyl) refers to a fully saturated cyclic hydrocarbon group having from 3 to 8 carbon atoms (e.g. 3 to 6 or 3 to 4 carbon atoms). The term encompasses cyclopropyl, cyclobutyl, cyclopentyl, cyclohexyl, cycloheptyl and cyclooctyl as well as bridged systems such as bicyclo[1.1.1]pentyl.

The term “4-7 membered heterocyclyl” (e.g. 4-6 membered heterocyclyl) refers to a non-aromatic cyclic group having 4 to 7 ring atoms (e.g. 4 to 6 ring atoms) and at least one heteroatom selected from N, O, S and B. The term “heterocyclyl” is interchangeable with “heterocyclic ring”. The term encompasses oxetanyl, thietanyl, azetidinyl, pyrrolidinyl, tetrahydrofuranyl, tetrahydrothienyl, tetrahydropyranyl, piperidinyl, piperazinyl, morpholinyl, thiomorpholinyl and homomorpholinyl. Other heterocyclyl groups, for example, 4-6 membered heterocyclyl and 4-5 membered heterocyclyl are as defined above but contain different numbers of ring atoms. 4-7 membered heterocyclyl groups can typically be substituted by one or more oxo groups. Suitably, thietanyl is substituted by one or two oxo groups. Bicyclic heterocyclic compounds are also encompassed, such as the following:

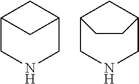

The term “hydroxy” (which may also be referred to as “hydroxyl”) refers to an —OH group.

The term “oxo” refers to a═O substituent, whereby an oxygen atom is doubly bonded to carbon (e.g. C═O) or another element (e.g. S═O, S(═O)₂). The carbon or other element is suitably an atom of an alkyl, cycloalkyl or heterocyclyl group.

The term “halo” refers to fluorine, chlorine, bromine or iodine. Particular examples of halo are fluorine and chlorine, especially fluorine.

The term “5-6 membered heteroaryl” refers to a cyclic group with aromatic character having 5-6 ring atoms, at least one of which is a heteroatom independently selected from N, O and S. The term encompasses pyrrolyl, furanyl, thienyl, imidazolyl, pyrazolyl, thiazolyl, oxadiazolyl, thiadiazolyl, triazolyl, oxazolyl, tetrazolyl, pyridyl, pyrimidinyl, pyradizinyl and pyrazinyl.

In one embodiment, R^{A1} is H. In a second embodiment, R^{A1} is C₁₋₄ alkyl such as methyl. In a third embodiment, R^{A1} is C₁₋₄ haloalkyl such as CF₃. In a fourth embodiment, R^{A1} is C≡CH.

In one embodiment, R^{A2} is H. In a second embodiment, R^{A2} is C₁₋₄ alkyl such as methyl. In a third embodiment, R^{A2} is C₁₋₄ haloalkyl such as CF₃.

In one embodiment, R^{A1} is C₁₋₄ alkyl such as methyl and R^{A2} is C₁₋₄ alkyl such as methyl. In a second embodiment, R^{A1} is H and R^{A2} is C₁₋₄ alkyl such as methyl. In a third embodiment, R^{A1} is C₁₋₄ haloalkyl such as CF₃ and R^{A2} is C₁₋₄ alkyl such as methyl. In a fourth embodiment, R^{A1} is H and R^{A2} is C₁₋₄ haloalkyl such as CF₃. In a fifth embodiment, R^{A1} is C≡CH and R^{A2} is H.

In one embodiment, and when R^{A1} and R^{A2} are different, the groups have the following stereochemical configuration:

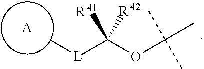

In another embodiment, and when R^{A1} and R^{A2} are different, the groups have the following stereochemical configuration:

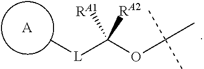

For example, when R^{A1} is CF₃ and R^{A2} is H, R^{A1} and R^{A2} have the above stereochemical configuration.

In one embodiment, R^{A1} and R^{A2} join to form a C₃₋₆ cycloalkyl ring:

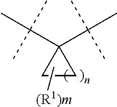

wherein m, n and R¹ are as defined herein.

Suitably, the C₃₋₆ cycloalkyl ring is cyclobutyl.

In one embodiment, R¹ is fluoro. In a second embodiment, R¹ is methyl. In a third embodiment, R¹ is cyano. In a fourth embodiment, R¹ is OCH₃. In a fifth embodiment, R¹ is CF₃. In a sixth embodiment, two R¹ groups which are attached to the same carbon atom join to form a C₃₋₄ cycloalkyl ring, e.g., a cyclobutyl ring.

In one embodiment, m is 0. In a second embodiment, m is 1. In a third embodiment, m is 2.

Suitably, m is 0.

In one embodiment, n is 1. In a second embodiment, n is 2. In a third embodiment, n is 3. In a fourth embodiment, n is 4. Suitably, n is 2.

Suitably, m is 2 and R¹ is fluoro.

When m is 2, suitably the two R¹ groups are attached to the same carbon. For example, when m is 2, and n is 2, such that a cyclobutyl ring forms, suitably the two R¹ groups are attached to the same carbon in the 3-position of the cyclobutyl ring.

Suitably, n is 2 and two R¹ groups which are attached to the same carbon atom join to form a cyclobutyl ring.

In one embodiment, R^{A1} and R^{A2} join to form a 4-6 membered heterocyclic ring, such as a 4-membered heterocyclic ring. The heterocyclic ring comprises one or more (such as one) heteroatom, such as O, N or S. A heteroatom is defined as an atom other than carbon or hydrogen. Suitably, the heteroatom is O or S.

In one embodiment, the 4-6 membered heterocyclic ring is not substituted. In another embodiment, the 4-6 membered heterocyclic ring is substituted by one or more (such as one, two or three, e.g. one) groups selected from oxo, fluoro or methyl.

In one embodiment, A is phenyl optionally substituted by one or more (such as one, two or three e.g. one) R². In a second embodiment, A is naphthyl optionally substituted by one or more (such as one, two or three e.g. one) R². In a third embodiment, A is 5-6 membered heteroaryl optionally substituted by one or more (such as one, two or three e.g. one) R². In some cases, in this embodiment, A is other than pyrazinyl and pyrazolyl. Suitably, A is other than pyrazolyl.

Suitably, A is phenyl optionally substituted by one or more (such as one, two or three e.g. one) R².

Alternatively, A is a 6 membered heteroaryl (such as pyridyl) optionally substituted by one or more (such as one, two or three e.g. one) R².

When A is phenyl, naphthyl or 6-membered heteroaryl, suitably one R² is in the 4-position with respect to C(R^{A1})(R^{A2}):

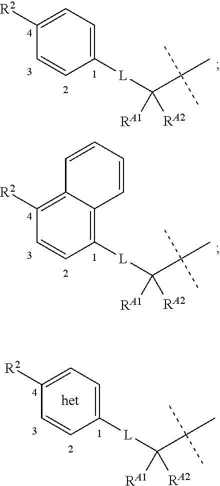

wherein

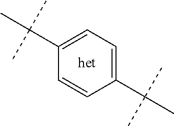

represents a 6-membered heteroaryl ring, the dashed lines indicate the bond connected to the rest of the compound of formula (I), and R^{A1}, R^{A2}, L and R² are as defined elsewhere herein. Although one R² is shown above, A is optionally substituted by one or more R² and thus there may be other R² groups attached to the ring.

Suitably, the R² group in the 4-position is other than fluoro.

In one embodiment A is not substituted. In a second embodiment, A is substituted by one or more (such as one, two or three e.g. one) R². Suitably, A is substituted by one R². Alternatively, A is substituted by two R². Alternatively, A is substituted by three R².

When A is substituted by two R², the two R² groups may be the same or different, suitably different.

When A is substituted by three R², the three R² groups may be the same or different, suitably different.

When A is substituted by two R², and R² is halo or C₁₋₄ alkyl e.g. methyl, the two R² groups are suitably the same.

When A is substituted by three R², and R² is halo or C₁₋₄ alkyl e.g. methyl, the three R² groups are suitably the same.

In one embodiment, R² is C₁₋₄ alkyl, such as methyl. In a second embodiment, R² is C₁₋₄ alkoxy such as methoxy. In a third embodiment, R² is C₁₋₄ haloalkyl such as CF₃. In a fourth embodiment, R² is C₁₋₄ haloalkoxy such as OCF₃. In a fifth embodiment, R² is hydroxy. In a sixth embodiment, R² is CO₂H. In a seventh embodiment, R² is cyano. In an eighth embodiment, R² is methanesulfonyl. In a ninth embodiment, R² is halo such as chloro or fluoro, e.g., chloro. In a tenth embodiment, R² is SF₅. In an eleventh embodiment, R² is SC₁₋₄ alkyl e.g. SCH₃. In a twelfth embodiment, R² is SC₁₋₄ haloalkyl e.g. SCF₃. In a thirteenth embodiment, R² is —C≡C—C₁₋₂haloalkyl such as —C≡C—CF₃. In a fourteenth embodiment, R² is phenyl, wherein the phenyl is optionally substituted by halo or C₁₋₂ haloalkyl. Suitably, R² is unsubstituted phenyl. Alternatively, R² is phenyl substituted by halo or C₁₋₂ haloalkyl, e.g., CF₃.

Suitably, C₁₋₄ haloalkyl is optionally substituted by phenyl. When C₁₋₄ haloalkyl is substituted by phenyl, the following structure may form:

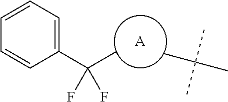

Suitably, one C—H group in the C₁₋₄ haloalkyl group is replaced with a C-phenyl group.

Most suitably, R² is CF₃.

In a fifteenth embodiment, taken together, two R² groups are attached to adjacent carbon atoms and are joined to form a C₃₋₈ cycloalkyl or 4-7 membered heterocyclic ring. Suitably, two R² groups are attached to adjacent carbon atoms and are joined to form a C₃₋₈ cycloalkyl ring. Suitably, two R² groups are attached to adjacent carbon atoms and are joined to form a 4-7 membered heterocyclic ring such as a 4-6 membered heterocyclic ring.

Suitably, A is substituted by one R² wherein R² is C₁₋₄ haloalkyl such as CF₃.

Suitably, when A is phenyl, L is a bond or C≡C, and R² is R^{2a}, R^{2b}, R^{2c}, R^{2d} and R^{2e}, the compound of formula (I) is:

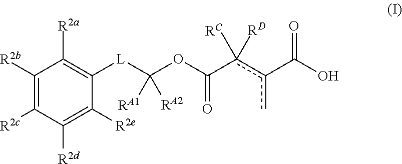

wherein:
   R^{2a} is H or halo;
   R^{2b} is H or halo;
   R^{2c} is C₁₋₄ alkyl, C₁₋₄ alkoxy, C₁₋₄ haloalkyl, C₁₋₄ haloalkoxy, cyano, Cl, Br, SF₅, SC₁₋₄ haloalkyl, —C≡C—C₁₋₂haloalkyl and phenyl, wherein the phenyl is optionally substituted by halo, or C₁₋₂ haloalkyl, and wherein the C₁₋₄ haloalkyl is optionally substituted by phenyl;
   R^{2d} is H or fluoro; and
   R^{2e} is H;
      wherein when R^{2a}, R^{2d} and R^{2e} are H, R^{2b} is Cl and R^{2c} is CF₃, R^{A2} is other than H.

In one embodiment, R^{2a} is H. In a second embodiment, R^{2a} is halo.

In one embodiment, R^{2b} is H. In a second embodiment, R^{2b} is halo.

In one embodiment, R^{2c} is C₁₋₄ alkyl such as methyl. In a second embodiment, R^{2c} is C₁₋₄ alkoxy such as OCH₃. In a third embodiment, R^{2c} is C₁₋₄ haloalkyl, such as CF₃, wherein C₁₋₄ haloalkyl is optionally substituted by phenyl. In a fourth embodiment, R^{2c} is C₁₋₄ haloalkoxy such as OCF₃. In a fifth embodiment, R^{2c} is cyano. In a sixth embodiment, R^{2c} is Cl. In a seventh embodiment, R^{2c} is Br. In an eighth embodiment, R^{2c} is SF₅. In a ninth embodiment, R^{2c} is SC₁₋₄ haloalkyl such as SCF₃. In a tenth embodiment, R^{2c} is —C≡C—C₁₋₂haloalkyl. In an eleventh embodiment, R^{2c} is phenyl, wherein the phenyl is optionally substituted by halo.

When R^{2a}, R^{2d}, R^{2d} and R^{2e} are H, R^{2b} is Cl and R^{2c} is CF₃, R^{A2} is other than H.

When A is naphthyl, R² is independently selected from the group consisting of C₁₋₄ alkyl, C₁_₄ alkoxy, C₁₋₄ haloalkyl, C₁₋₄ haloalkoxy, hydroxy, CO₂H, cyano, methanesulfonyl, chloro, bromo, SF₅, SC₁₋₄ alkyl, SC₁₋₄ haloalkyl, —C≡C—C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring.

When A is thienyl, R² is C₁₋₄ haloalkyl such as CF₃.

In one embodiment, L is a bond. In a second embodiment, L is C₁₋₂alkylene, e.g. CH₂. In a third embodiment, L is C≡C.

In one embodiment, R^{C} is H. In one embodiment, R^{C} is C₁₋₂ alkyl, in particular methyl. In one embodiment, R^{C} is hydroxy. In one embodiment, R^{C} is fluoro. In one embodiment, R^{C} is methoxy.

In one embodiment, R^{D} is H. In one embodiment, R^{D} is C₁₋₂ alkyl, in particular methyl. In one embodiment, R^{D} is hydroxy. In one embodiment, R^{D} is fluoro. In one embodiment, R^{D} is methoxy.

In one embodiment, taken together, R^{C} and R^{D} combine to form a C₃₋₄ cycloalkyl ring, e.g., a cyclopropyl ring.

In one embodiment, R^{C} is H, C₁₋₂ alkyl (in particular methyl), hydroxy or fluoro; and R^{D} is H, C₁₋₂ alkyl (in particular methyl), or fluoro. In one embodiment, R^{C} is H, C₁₋₂ alkyl (in particular methyl), hydroxy or fluoro; and R^{D} is H, C₁₋₂ alkyl (in particular methyl) or fluoro. In one embodiment, R^{C} is H, C₁₋₂ alkyl (in particular methyl), hydroxy or fluoro; and R^{D} is H or C₁₋₂ alkyl (in particular methyl). In one embodiment, R^{C} is H, C₁₋₂ alkyl (in particular methyl), hydroxy or fluoro; and R^{D} is H or fluoro. In one embodiment, R^{C} is H, C₁₋₂ alkyl (in particular methyl), hydroxy or fluoro; and R^{D} is H. In one embodiment, R^{C} is H and R^{D} is H or C₁₋₂ alkyl (in particular methyl). In one embodiment, R^{C} is H and R^{D} is H or fluoro. In one embodiment, R^{C} is H or C₁₋₂ alkyl (in particular methyl); and R^{D} is H, C₁₋₂ alkyl (in particular methyl), or fluoro. In one embodiment, R^{C} is H or C₁₋₂ alkyl (in particular methyl); and R^{D} is H or C₁₋₂ alkyl (in particular methyl). In one embodiment, R^{C} is H or C₁₋₂ alkyl (in particular methyl); and R^{D} is H. In one embodiment, R^{C} is H and R^{D} is H. In one embodiment, both of R^{C} and R^{D} are not hydroxy. In one embodiment, R^{C} is methoxy and R^{D} is H.

In one embodiment, the compound of formula (I) is:

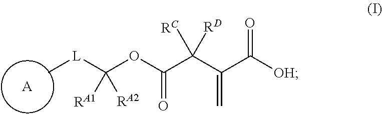

or a pharmaceutically acceptable salt and/or solvate thereof;
wherein A, L, R^{A1}, R^{A2}, R^{C} and R^{D} are as defined elsewhere herein. The carbon-carbon double bond in this structure is referred to as “exo”.

In another embodiment, the compound of formula (I) is:

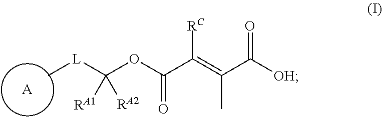

or a pharmaceutically acceptable salt and/or solvate thereof;
wherein A, L, R^{A1}, R^{A2} and R^{C} are as defined elsewhere herein. The carbon-carbon double bond in this structure is referred to as “endo”.

In the endo embodiment, the double bond may be cis or trans such that both of the following moieties are covered:

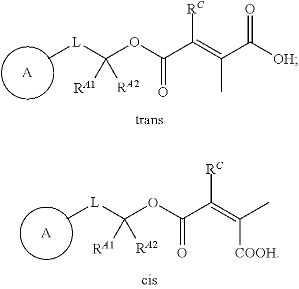

Similarly, as used herein, the following structure:

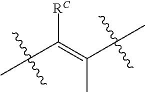

encompasses both cis and trans isomers:

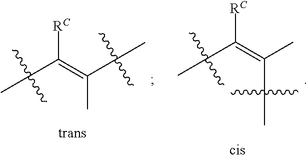

Suitably, the endo double bond in the compound of formula (I) is trans.

Typically, e.g., as shown in the Biological Examples section, the compounds of formula (I) in which the carbon-carbon double bond is exo are more potent (e.g., have a lower IC₅₀, lower EC₅₀ and/or higher Eₘₐₓ in the assays described herein) than the equivalent compounds of formula (I) in which the carbon-carbon double bond is endo.

The compounds of formula (I) in which the carbon-carbon double bond is endo can generally be obtained by isomerisation from compounds of formula (I) in which the carbon-carbon double bond is exo and such isomerisation may occur in in vitro assays or in vivo following administration of the exo compound. In some cases, isomerisation in in vitro assays, such as in vitro hepatocyte stability assays, or in vivo following administration of the exo compound, may be partial and thus lead to a mixture of the endo and exo compound resulting. In some cases, the mixture of endo and exo isomers may contribute to the activity observed in a particular assay. Suitably, compounds of formula (I), such as those in which the carbon-carbon double bond is exo, are stable to isomerisation.

In one embodiment,

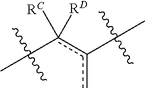

represents

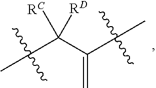

R^{A1} and R^{A2} join to form an oxetane ring, A is phenyl which is substituted by one R² wherein R² is CF₃, and R^{C} and R^{D} are H.

In one embodiment,

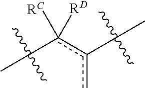

represents

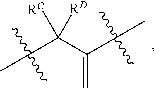

R^{A1} and R^{A2} join to form a thietane ring, A is phenyl which is substituted by one R² wherein R² is CF₃, and R^{C} and R^{D} are H.

In one embodiment,

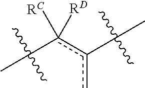

represents

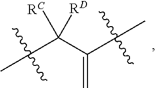

R^{A1} and R^{A2} join to form a cyclobutyl ring, A is 2-pyridyl which is substituted by one R² wherein R² is CF₃, and R^{C} and R^{D} are H.

In one embodiment, there is provided a compound of formula (Id):

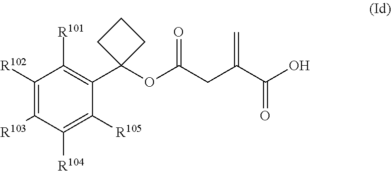

wherein:
R¹⁰¹ is H or Cl;
R¹⁰² is H or halo;
R¹⁰³ is H, halo, CH₃, CF₃ or OCF₃;
R¹⁰⁴ is H, halo or CF₃;
R¹⁰⁵ is H or Cl;
   wherein one, two or three of any of the R¹⁰¹ to R¹⁰⁵ groups are other than H and the remainder are H;
   or a pharmaceutically acceptable salt and/or solvate thereof.

In one embodiment, R¹⁰¹ is H. In a second embodiment, R¹⁰¹ is C.

In one embodiment, R¹⁰² is H. In a second embodiment, R¹⁰² is halo, e.g., C.

In one embodiment, R¹⁰³ is H. In a second embodiment, R¹⁰³ is halo, e.g., C or Br. In a third embodiment, R¹⁰³ is CH₃. In a fourth embodiment, R¹⁰³ is CF₃. In a fifth embodiment, R¹⁰³ is OCF₃.

In one embodiment, R¹⁰⁴ is H. In a second embodiment, R¹⁰⁴ is halo, e.g. C., In a third embodiment, R¹⁰⁴ is CF₃.

In one embodiment, R¹⁰⁵ is H. In a second embodiment, R¹⁰⁵ is C.

In one embodiment, there is provided a compound of formula (Ie):

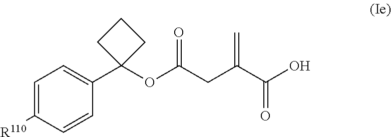

wherein:
R¹⁰ is SF₅, SC₁₋₄alkyl or SC₁₋₄haloalkyl;
   or a pharmaceutically acceptable salt and/or solvate thereof.

In one embodiment, R¹⁰ is SF₅. In a second embodiment, R¹⁰ is SC₁₋₄alkyl e.g. SCH₃. In a third embodiment, R¹⁰ is SC₁₋₄haloalkyl e.g. SCF₅.

In one embodiment there is provided a compound of formula (I), which is: 2-methylene-4-oxo-4-(1-(4-(trifluoromethyl)phenyl)cyclobutoxy)butanoic acid; or a pharmaceutically acceptable salt and/or solvate of any one thereof.

In another embodiment there is provided a compound of formula (I), which is selected from the group consisting of:
2-methylene-4-oxo-4-((2-(4-(trifluoromethyl)phenyl)propan-2-yl)oxy)butanoic acid;
(R)-2-methylene-4-oxo-4-(1-(4-(trifluoromethyl)phenyl)ethoxy)butanoic acid;
(S)-2-methylene-4-oxo-4-(1-(4-(trifluoromethyl)phenyl)ethoxy)butanoic acid;
4-(1-(4-bromophenyl)cyclobutoxy)-2-methylene-4-oxobutanoic acid;
4-(1-(2-chloro-4-(trifluoromethyl)phenyl)cyclobutoxy)-2-methylene-4-oxobutanoic acid;
2-methylene-4-oxo-4-((1-(4-(trifluoromethyl)phenyl)cyclopentyl)oxy)butanoic acid;
4-(1-(4-chlorophenyl)cyclobutoxy)-2-methylene-4-oxobutanoic acid;
2-methylene-4-oxo-4-(1-(4-((trifluoromethyl)thio)phenyl)cyclobutoxy)butanoic acid;
2-methylene-4-oxo-4-(1-(4-(trifluoromethyl)phenyl)propoxy)butanoic acid;
2-methylene-4-oxo-4-(1-(4-(trifluoromethyl)phenyl)cyclopropoxy)butanoic acid;
4-(1-(3-chloro-4-(trifluoromethyl)phenyl)cyclobutoxy)-2-methylene-4-oxobutanoic acid;
2-methylene-4-oxo-4-((4-(trifluoromethyl)benzyl)oxy)butanoic acid;
4-(1-(3-chloro-4-methylphenyl)cyclobutoxy)-2-methylene-4-oxobutanoic acid;
2-methylene-4-oxo-4-(1-(4-(trifluoromethoxy)phenyl)cyclobutoxy)butanoic acid;
4-(1-(4′-fluoro-[1,1-biphenyl]-4-yl)cyclobutoxy)-2-methylene-4-oxobutanoic acid;
4-(1-(3-fluoro-4-(trifluoromethyl)phenyl)cyclobutoxy)-2-methylene-4-oxobutanoic acid;
2-methylene-4-oxo-4-(1-(4-(pentafluoro-I⁶-sulfaneyl)phenyl)cyclobutoxy)butanoic acid;
4-(1-(4-(1,1-difluoroethyl)phenyl)cyclobutoxy)-2-methylene-4-oxobutanoic acid;
4-(1-(4-(difluoromethyl)phenyl)cyclobutoxy)-2-methylene-4-oxobutanoic acid;
(E)-2-methyl-4-oxo-4-(1-(4-(trifluoromethyl)phenyl)cyclobutoxy)but-2-enoic acid;
2-methylene-4-oxo-4-(1-(6-(trifluoromethyl)pyridin-3-yl)cyclobutoxy)butanoic acid;
4-((3-(4-bromophenyl)oxetan-3-yl)oxy)-2-methylene-4-oxobutanoic acid;
2-methylene-4-oxo-4-((3-(4-(trifluoromethyl)phenyl)oxetan-3-yl)oxy)butanoic acid;
4-(1-(4-cyanophenyl)cyclobutoxy)-2-methylene-4-oxobutanoic acid;
2-methylene-4-oxo-4-(1-(4′-(trifluoromethyl)-[1,1′-biphenyl]-4-yl)cyclobutoxy)butanoic acid;
2-methylene-4-oxo-4-(1-(5-(trifluoromethyl)pyridin-2-yl)cyclobutoxy)butanoic acid;
4-(1-(4-(difluoro(phenyl)methyl)phenyl)cyclobutoxy)-2-methylene-4-oxobutanoic acid;
4-(3,3-difluoro-1-(4-(trifluoromethyl)phenyl)cyclobutoxy)-2-methylene-4-oxobutanoic acid;
3,3-dimethyl-2-methylene-4-oxo-4-(1-(4-(trifluoromethyl)phenyl)cyclobutoxy) butanoic acid;
2-methylene-4-oxo-4-(1-(4-(perfluoroethyl)phenyl)cyclobutoxy)butanoic acid;
2-methylene-4-oxo-4-(1-(5-(trifluoromethyl)thiophen-2-yl)cyclobutoxy)butanoic acid;
(S)-2-methylene-4-oxo-4-(2,2,2-trifluoro-1-(4-(trifluoromethyl)phenyl)ethoxy)butanoic acid;
(R)-2-methylene-4-oxo-4-(2,2,2-trifluoro-1-(4-(trifluoromethyl)phenyl)ethoxy)butanoic acid;
2-methylene-4-oxo-4-((2-(4-(trifluoromethyl)phenyl)spiro[3.3]heptan-2-yl)oxy)butanoic acid;
2-methylene-4-oxo-4-(1-(4-(3,3,3-trifluoroprop-1-yn-1-yl)phenyl)cyclobutoxy)butanoic acid;
2-methylene-4-oxo-4-((1-(4-(trifluoromethyl) phenyl)prop-2-yn-1-yl)oxy)butanoic acid (Enantiomer 1);
2-methylene-4-oxo-4-((1-(4-(trifluoromethyl) phenyl)prop-2-yn-1-yl)oxy)butanoic acid (Enantiomer 2);
2-(1-((1-(4-(trifluoromethyl)phenyl)cyclobutoxy)carbonyl)cyclopropyl)acrylic acid;
2-methylene-4-oxo-4-((3-(4-(trifluoromethyl)phenyl)thietan-3-yl)oxy)butanoic acid; and
4-((1s,3s)-3-cyano-1-(4-(trifluoromethyl)phenyl)cyclobutoxy)-2-methylene-4-oxobutanoic acid;
or a pharmaceutically acceptable salt and/or solvate of any one thereof.

In another embodiment there is provided a compound of formula (I), which is selected from the group consisting of:
2-methylene-4-oxo-4-((2-(4-(trifluoromethyl)phenyl)propan-2-yl)oxy)butanoic acid;
(R)-2-methylene-4-oxo-4-(1-(4-(trifluoromethyl)phenyl)ethoxy)butanoic acid;
(S)-2-methylene-4-oxo-4-(1-(4-(trifluoromethyl)phenyl)ethoxy)butanoic acid;
4-(1-(4-bromophenyl)cyclobutoxy)-2-methylene-4-oxobutanoic acid;
4-(1-(2-chloro-4-(trifluoromethyl)phenyl)cyclobutoxy)-2-methylene-4-oxobutanoic acid;
2-methylene-4-oxo-4-((1-(4-(trifluoromethyl)phenyl)cyclopentyl)oxy)butanoic acid;
4-(1-(4-chlorophenyl)cyclobutoxy)-2-methylene-4-oxobutanoic acid;
2-methylene-4-oxo-4-(1-(4-((trifluoromethyl)thio)phenyl)cyclobutoxy)butanoic acid;
2-methylene-4-oxo-4-(1-(4-(trifluoromethyl)phenyl)propoxy)butanoic acid;
2-methylene-4-oxo-4-(1-(4-(trifluoromethyl)phenyl)cyclopropoxy)butanoic acid;
4-(1-(3-chloro-4-(trifluoromethyl)phenyl)cyclobutoxy)-2-methylene-4-oxobutanoic acid;
2-methylene-4-oxo-4-((4-(trifluoromethyl)benzyl)oxy)butanoic acid;
4-(1-(3-chloro-4-methylphenyl)cyclobutoxy)-2-methylene-4-oxobutanoic acid;
2-methylene-4-oxo-4-(1-(4-(trifluoromethoxy)phenyl)cyclobutoxy)butanoic acid;
4-(1-(4′-fluoro-[1,1-biphenyl]-4-yl)cyclobutoxy)-2-methylene-4-oxobutanoic acid;
4-(1-(3-fluoro-4-(trifluoromethyl)phenyl)cyclobutoxy)-2-methylene-4-oxobutanoic acid;
2-methylene-4-oxo-4-(1-(4-(pentafluoro-I⁶-sulfaneyl)phenyl)cyclobutoxy)butanoic acid;
4-(1-(4-(1,1-difluoroethyl)phenyl)cyclobutoxy)-2-methylene-4-oxobutanoic acid;
4-(1-(4-(difluoromethyl)phenyl)cyclobutoxy)-2-methylene-4-oxobutanoic acid;
(E)-2-methyl-4-oxo-4-(1-(4-(trifluoromethyl)phenyl)cyclobutoxy)but-2-enoic acid;
2-methylene-4-oxo-4-(1-(6-(trifluoromethyl)pyridin-3-yl)cyclobutoxy)butanoic acid;
4-((3-(4-bromophenyl)oxetan-3-yl)oxy)-2-methylene-4-oxobutanoic acid;
2-methylene-4-oxo-4-((3-(4-(trifluoromethyl)phenyl)oxetan-3-yl)oxy)butanoic acid;
4-(1-(4-cyanophenyl)cyclobutoxy)-2-methylene-4-oxobutanoic acid;
2-methylene-4-oxo-4-(1-(4′-(trifluoromethyl)-[1,1′-biphenyl]-4-yl)cyclobutoxy)butanoic acid;
2-methylene-4-oxo-4-(1-(5-(trifluoromethyl)pyridin-2-yl)cyclobutoxy)butanoic acid;
4-(1-(4-(difluoro(phenyl)methyl)phenyl)cyclobutoxy)-2-methylene-4-oxobutanoic acid;
4-(3,3-difluoro-1-(4-(trifluoromethyl)phenyl)cyclobutoxy)-2-methylene-4-oxobutanoic acid;
3,3-dimethyl-2-methylene-4-oxo-4-(1-(4-(trifluoromethyl)phenyl)cyclobutoxy) butanoic acid;
2-methylene-4-oxo-4-(1-(4-(perfluoroethyl)phenyl)cyclobutoxy)butanoic acid;
2-methylene-4-oxo-4-(1-(5-(trifluoromethyl)thiophen-2-yl)cyclobutoxy)butanoic acid;
(R)-2-methylene-4-oxo-4-(2,2,2-trifluoro-1-(4-(trifluoromethyl) phenyl)ethoxy)butanoic acid;
2-methylene-4-oxo-4-((2-(4-(trifluoromethyl)phenyl)spiro[3.3]heptan-2-yl)oxy)butanoic acid;
2-methylene-4-oxo-4-(1-(4-(3,3,3-trifluoroprop-1-yn-1-yl)phenyl)cyclobutoxy)butanoic acid;
2-methylene-4-oxo-4-((1-(4-(trifluoromethyl) phenyl)prop-2-yn-1-yl)oxy)butanoic acid (Enantiomer 1);
2-methylene-4-oxo-4-((1-(4-(trifluoromethyl)phenyl)prop-2-yn-1-yl)oxy)butanoic acid (Enantiomer 2);
2-(1-((1-(4-(trifluoromethyl)phenyl)cyclobutoxy)carbonyl)cyclopropyl)acrylic acid;
2-methylene-4-oxo-4-((3-(4-(trifluoromethyl)phenyl)thietan-3-yl)oxy)butanoic acid; and
4-((1s,3s)-3-cyano-1-(4-(trifluoromethyl)phenyl)cyclobutoxy)-2-methylene-4-oxobutanoic acid;
or a pharmaceutically acceptable salt and/or solvate of any one thereof.

In another embodiment there is provided a compound of formula (I), which is selected from the group consisting of:
4-(1-(3,5-difluoro-4-(trifluoromethyl)phenyl)cyclobutoxy)-2-methylene-4-oxobutanoic acid;
2-methylene-4-oxo-4-(1-((4-(trifluoromethyl)phenyl)ethynyl) cyclobutoxy)butanoic acid;
2-methylene-4-oxo-4-((5-(4-(trifluoromethyl)phenyl)spiro[2.3]hexan-5-yl)oxy)butanoic acid;
2-methylene-4-oxo-4-((4-(4-(trifluoromethyl)phenyl)tetrahydro-2H-pyran-4-yl)oxy)butanoic acid;
4-((1,1-dioxido-3-(4-(trifluoromethyl)phenyl)thietan-3-yl)oxy)-2-methylene-4-oxobutanoic acid;
4-((1-methyl-3-(4-(trifluoromethyl)phenyl)azetidin-3-yl)oxy)-2-methylene-4-oxobutanoic acid;
2-methylene-4-oxo-4-(3-(5-(trifluoromethyl)pyridin-2-yl)oxetan-3-yloxy)butanoic acid;
4-(3-methyl-1-(4-(trifluoromethyl)phenyl)cyclobutoxy)-2-methylene-4-oxobutanoic acid;
4-(3-methyl-1-(4-(trifluoromethyl)phenyl)cyclobutoxy)-2-methylene-4-oxobutanoic acid;
2-methylene-4-oxo-4-((5-(trifluoromethyl)pyridin-2-yl)methoxy)butanoic acid;
4-(1-(5-chloropyridin-2-yl)cyclobutoxy)-2-methylene-4-oxobutanoic acid;
4-((3-fluoro-4-(trifluoromethyl)benzyl)oxy)-2-methylene-4-oxobutanoic acid;
4-(1-(5-bromopyridin-2-yl)cyclobutoxy)-2-methylene-4-oxobutanoic acid;
4-(1-(6-bromonaphthalen-2-yl)cyclobutoxy)-2-methylene-4-oxobutanoic acid;
2-methylene-4-oxo-4-(3-(trifluoromethyl)-1-(4-(trifluoromethyl)phenyl)cyclobutoxy)butanoic acid;
2-methylene-4-oxo-4-((2-(5-(trifluoromethyl)pyridin-2-yl)propan-2-yl)oxy)butanoic acid;
4-(3-methoxy-1-(4-(trifluoromethyl)phenyl)cyclobutoxy)-2-methylene-4-oxobutanoic acid;
3,3-dimethyl-2-methylene-4-oxo-4-(1-(5-(trifluoromethyl)pyridin-2-yl)cyclobutoxy)butanoic acid;
2-methylene-4-oxo-4-(1-(5-(trifluoromethyl)pyrimidin-2-yl)cyclobutoxy)butanoic acid; and
2-methylene-4-oxo-4-(1-(5-(trifluoromethyl)pyridin-2-yl)cyclopropoxy)butanoic acid;
or a pharmaceutically acceptable salt and/or solvate of any one thereof.

In another embodiment there is provided a compound of formula (I), which is selected from the group consisting of:
2-methylene-4-oxo-4-(1-(6-(trifluoromethyl)pyridazin-3-yl)cyclobutoxy)butanoic acid; and
2-methylene-4-oxo-4-(1-(5-(trifluoromethyl)pyrazin-2-yl)cyclobutoxy)butanoic acid;
or a pharmaceutically acceptable salt and/or solvate of any one thereof.

Compounds and intermediates of the invention may be made using the schemes below and as set out in the Example section below.

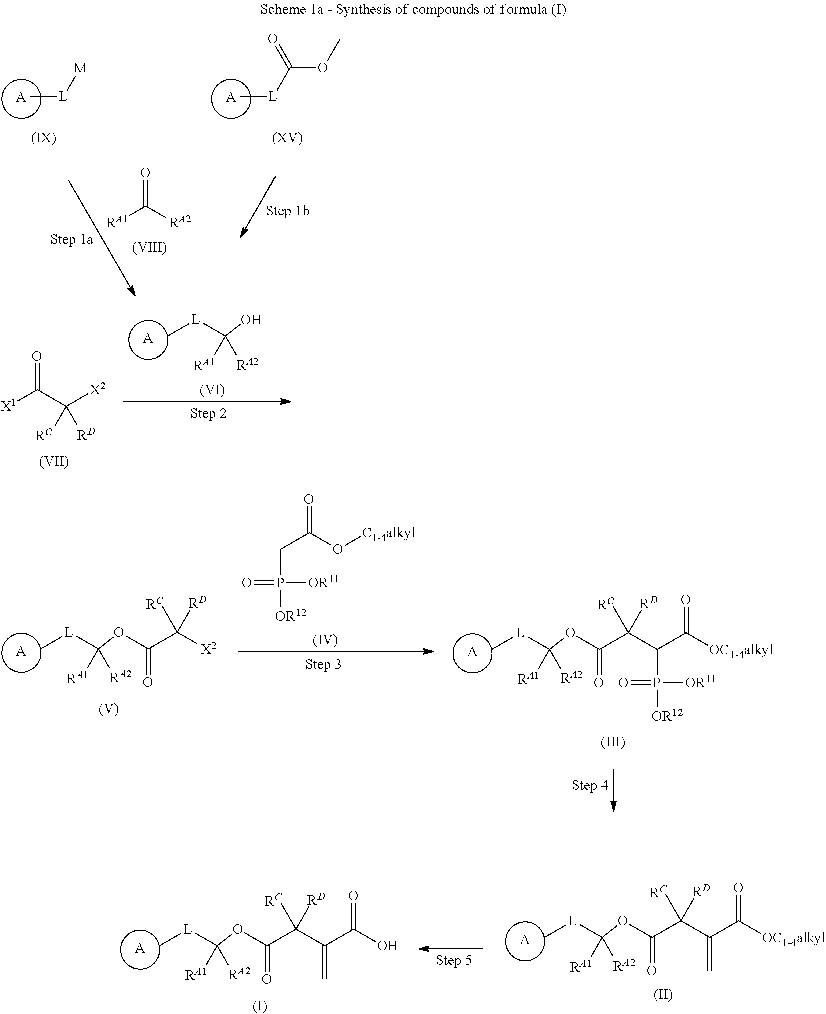

A, L, R^{A1}, R^{A2}, R^{C} and R^{D} are defined elsewhere herein and the C₁₋₄alkyl group in compounds of formula (II), (Ill) and (IV) is optionally substituted by halo. The synthetic detail for steps 1 to 5 are described under Scheme 1b below. Compounds of formulae (Ib), (Ic), (Id), (le), (If) and (Ig) may also be made via this route.

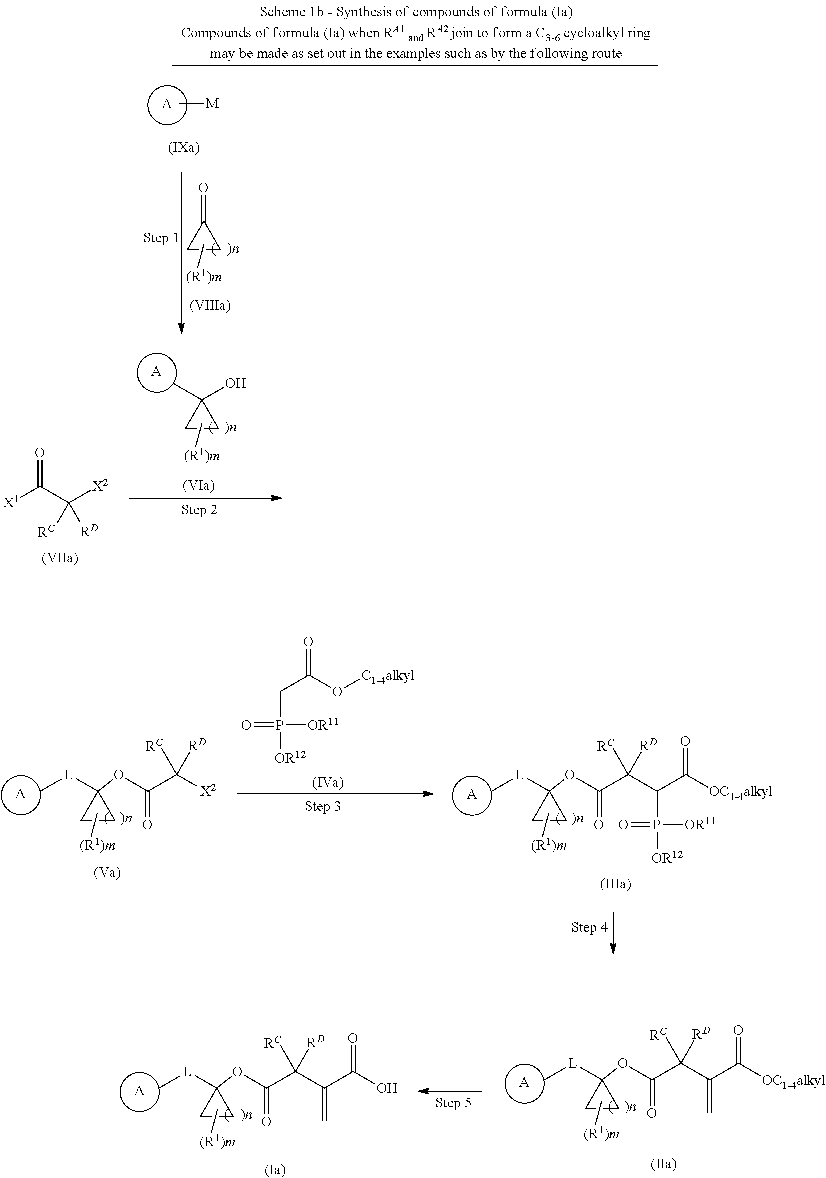

R¹, A, R^{C}, R^{D}, m and n are as defined elsewhere herein.

Step 1 and Step 1a: Grignard reagent ((IX) such as (IXa); M=MgBr or MgCl) or aryllithium ((IX) such as (IXa); M=Li)—prepared from the corresponding bromide or iodide—is reacted with cyclic or acyclic ketone (VIII), such as (VIIIa), to give compounds of formula (VI), such as (VIa).

Step 1b: Certain compounds of formula (VI) may also be made by Grignard addition (e.g. C₁₋₄ alkylMgBr e.g., MeMgBr) to methyl ester (XV).

Step 2: Alcohol (VI), such as (VIa), is condensed with a compound of formula (VII), such as (VIIa), wherein X¹ and X² represent leaving groups, such as halo, e.g., chloro, bromo or iodo, to give monoester (V), such as (Va).

Step 3: Monoester (V), such as (Va), is reacted with a trialkylphosphonoacetate of formula (IV), such as (IVa), wherein R¹¹ and R¹² independently represent C₁₋₄ alkyl optionally substituted with halo, to provide a compound of formula (III), such as (IIIa).

Step 4: Condensation of a compound of formula (III), such as (IIIa), with formaldehyde or a formaldehyde equivalent thereof, e.g., paraformaldehyde, provides diesters of formula (II), such as (IIa).

Step 5: Basic hydrolysis (such as by using aqueous LiOH in THF) of the C₁₋₄ alkyl ester in compounds of formula (II), such as (IIa), provides the compound of formula (I), such as (Ia).

Compounds of formula (I) wherein R^{A1} is H, C₁₋₄ alkyl or C₁₋₄ haloalkyl and R^{A2} is C₁₋₄ alkyl or C₁₋₄ haloalkyl may be made using analogous methods wherein the compound of formula (VIII), such as (VIIIa), is replaced by the corresponding ketone or aldehyde. For certain compounds of formula (I) wherein R^{A1} is H, C₁₋₄ alkyl or C₁₋₄ haloalkyl and R^{A2} is C₁₋₄ alkyl or C₁₋₄ haloalkyl, the corresponding alcohol of formula (VI), such as (VIa), is commercially available. Certain starting materials used in the preparation of compounds of formula (I) wherein R^{A1} is H, C₁₋₄ alkyl or C₁₋₄ haloalkyl and R^{A2} is C₁₋₄ alkyl or C₁₋₄ haloalkyl may be made by reacting A-C(═O)H with C₁₋₄ alkylMgBr, such as MeMgBr, or by reacting A-COC₁₋₄alkyl or A-COC₁₋₄haloalkyl with C₁₋₄ alkylMgBr, such as MeMgBr, or by reducing A-COC₁₋₄alkyl or A-COC₁₋₄haloalkyl with NaBH₄.

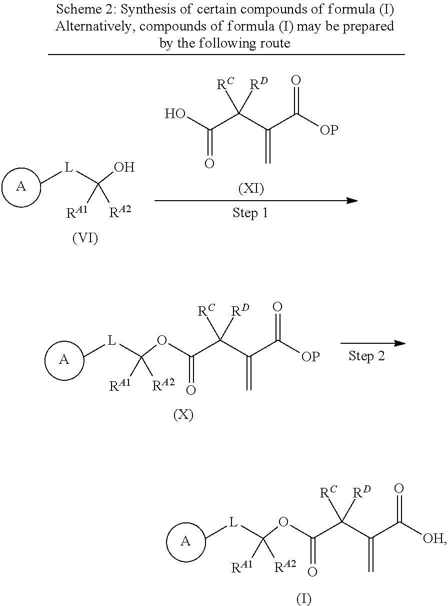

wherein P is a carboxylic acid protecting group such as C₁₋₆ alkyl, e.g., tert-butyl, C₁₋₆ haloalkyl, e.g., CH₂CCl₃, or para-methoxybenzyl and A, L, R^{A1}, R^{A2}, R^{C} and R^{D} are as defined elsewhere herein.

Step 1: Condensation of a commercially available compound of formula (VI) with a compound of formula (XI) under conditions known to the person skilled in the art (such as EDC·HCl or DCC in the presence of DMAP in DCM) provides esters of formula (X). Compounds of formula (VI) can also be made using the route set out in Scheme 1a.

Step 2: Carboxylic acid protecting group P may be removed using conditions known to the person skilled in the art. For example, C₁₋₆ alkyl, e.g., tert-butyl, or para-methoxybenzyl may be removed under acidic conditions, such as TFA in DCM, and C₁₋₆ haloalkyl, e.g., CH₂CCl₃ may be removed by using Zn powder in acetic acid to provide the compounds of formula (I).

There may be additional steps (e.g., one additional step) after the formation of compounds of formula (X) in order to manipulate groups within the certain compounds. For example, when R^{A1} and R^{A2} join to form a thietanyl ring, the sulfur atom of the thietanyl ring may be oxidised (e.g., using mCPBA in DCM) to provide the equivalent sulfone moiety, before deprotection in Step 2 to give compounds of formula (I).

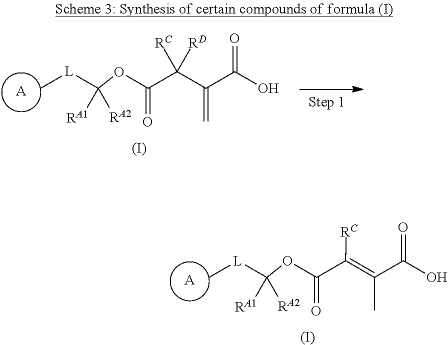

wherein A, L, R^{A1}, R^{A2} and R^{C} are defined elsewhere herein.

Step 1: Certain compounds of formula (I) may be obtained by isomerisation of certain other compounds of formula (I) under basic conditions, for example, using an organic base, such as diethylamine. Other organic bases suitably for the reaction are known to the skilled person.

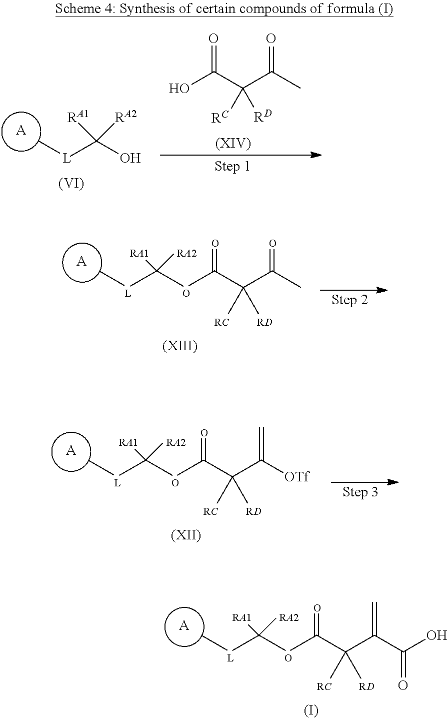

wherein A, L, R^{A1}, R^{A2}, R^{C} and R^{D} are defined elsewhere herein.

Step 1: Alcohols of formula (VI) undergo a coupling reaction with acids of formula (XIV) under standard coupling conditions, such as DCC and DMAP in DCM, to give compounds of formula (XIII).

Step 2: Compounds of formula (XIII) undergo a triflation reaction at reduced (<0° C.) temperatures in the presence of a base, such as LDA, and a triflation agent, such as phenyl triflimide, to give triflates of formula (XII).

Step 3: Triflates of formula (XII) undergo a transition metal-catalysed carbonylation reaction (e.g., CO, in the presence of a palladium catalyst, base and AcOH) to provide compounds of formula (I).

The skilled person will appreciate that protecting groups may be used throughout the synthetic schemes described herein to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10: 0471697540. Examples of nitrogen protecting groups include trityl (Tr), tert-butyloxycarbonyl (BOC), 9-fluorenylmethyloxycarbonyl (Fmoc), acetyl (Ac), benzyl (Bn) and para-methoxy benzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters). Specific examples of carboxylic acid protecting groups include alkyl esters (such as C₁₋₆ alkyl e.g. C₁₋₄ alkyl esters), benzyl esters and silyl esters.

Thus, in one embodiment there is provided a process for preparing a compound of formula (I) or a salt such as a pharmaceutically acceptable salt thereof, which comprises hydrolysing a compound of formula (II):

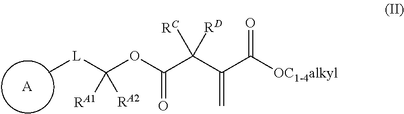

wherein the C₁₋₄alkyl group is optionally substituted by halo, and A, L, R^{A1}, R^{A2}, R^{C} and R^{D} are defined elsewhere herein; or a salt thereof.

Suitably, there is provided a process for preparing a compound of formula (Ia) or a salt such as a pharmaceutically acceptable salt thereof, which comprises hydrolysing a compound of formula (IIa):

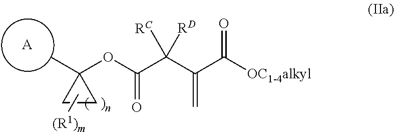

wherein A, R¹, m, n, R^{C} and R^{D} are defined elsewhere herein; or a salt thereof.

In one embodiment, there is provided a process for preparing a compound of formula (I) or a salt such as a pharmaceutically acceptable salt thereof, which comprises deprotecting a compound of formula (X):

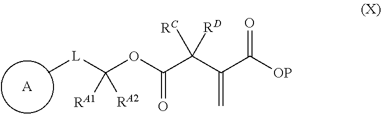

or a salt thereof; wherein A, L, R^{A1}, R^{A2}, R^{C}, R^{D} and P are as defined herein.

In one embodiment, there is provided a process for preparing a compound of formula (I) or a salt such as a pharmaceutically acceptable salt thereof, which comprises reacting a compound of formula (XII):

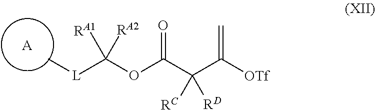

or a salt thereof;
with carbon monoxide under transition metal-catalysed conditions;
wherein A, L, R^{A1}, R^{A2}, R^{C} and R^{D} are as defined herein.

In one embodiment, there is provided a compound of formula (II):

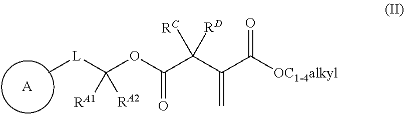

or a salt thereof, wherein the C₁₋₄alkyl group is optionally substituted by halo, and A, L, R^{A1}, R^{A2}, R^{C} and R^{D} are defined elsewhere herein.

Suitably, there is provided a compound of formula (IIa):

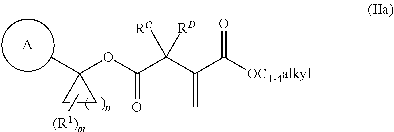

or a salt thereof, wherein A, R¹, m, n, R^{C} and R^{D} are defined elsewhere herein.

In one embodiment, there is provided a compound of formula (III):

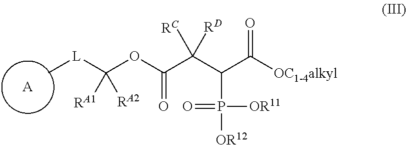

or a salt thereof, wherein the C₁₋₄alkyl group is optionally substituted by halo, and A, L, R^{A1}, R^{A2}, R^{C}, R^{D}, R¹¹ and R¹² are defined elsewhere herein.

Suitably, there is provided a compound of formula (IIIa):

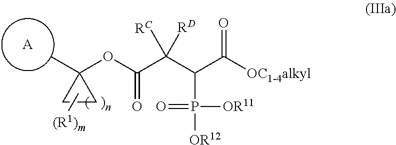

or a salt thereof, wherein A, R¹, m, n, R^{C}, R^{D}, R¹¹ and R¹² are defined elsewhere herein.

In one embodiment, there is provided a compound of formula (V):

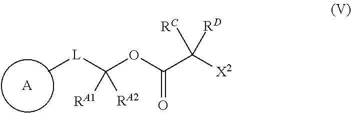

or a salt thereof, wherein A, L, R^{A1}, R^{A2}, R^{C}, R^{D} and X² are defined elsewhere herein.

Suitably, there is provided a compound of formula (Va):

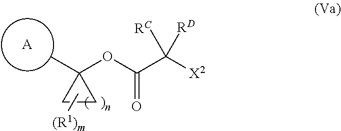

or a salt thereof, wherein A, R¹, m, n, R^{C}, R^{D} and X² are defined elsewhere herein.

Suitably, there is provided a compound of formula (X):

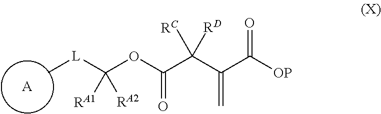

or a salt thereof, wherein A, L, R^{A1}, R^{A2}, R^{C}, R^{D} and P are as defined elsewhere.

In one embodiment, there is provided a compound of formula (XII):

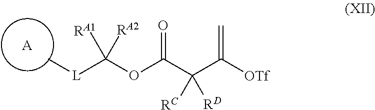

or a salt thereof, wherein A, L, R^{A1}, R^{A2}, R^{C} and R^{D} are as defined herein.

It will be appreciated that for use in therapy the salts of the compounds of formula (I) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids e.g. hydrochloric acid, hydrobromic acid, sulfuric acid, nitric acid or phosphoric acid. Also included are salts formed with organic acids e.g. succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts e.g. oxalates or formates, may be used, for example in the isolation of compounds of formula (I) and are included within the scope of this invention, as are basic addition salts such as sodium, potassium, calcium, aluminium, zinc, magnesium and other metal salts.

Pharmaceutically acceptable salts may also be formed with organic bases such as basic amines e.g. with ammonia, meglumine, tromethamine, piperazine, arginine, choline, diethylamine, benzathine or lysine. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

Suitably, the compound of formula (I) is not a salt e.g. is not a pharmaceutically acceptable salt.

Suitably, where the compound of formula (I) is in the form of a salt, the pharmaceutically acceptable salt is a basic addition salt such as a carboxylate salt formed with a group 1 metal (e.g. a sodium or potassium salt), a group 2 metal (e.g. a magnesium or calcium salt) or an ammonium salt of a basic amine (e.g. an NH₄⁺ salt), such as a sodium salt.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water). Suitably, the compound of formula (I) is not a solvate.

The invention extends to a pharmaceutically acceptable derivative thereof, such as a pharmaceutically acceptable prodrug of compounds of formula (I). Typical prodrugs of compounds of formula (I) which comprise a carboxylic acid include ester (e.g. C₁₋₆ alkyl e.g. C₁₋₄ alkyl ester) derivatives thereof. Thus, in one embodiment, the compound of formula (I) is provided as a pharmaceutically acceptable prodrug. In another embodiment, the compound of formula (I) is not provided as a pharmaceutically acceptable prodrug.

Certain compounds of formula (I) may metabolize under certain conditions. Without wishing to be bound by theory, formation of an active metabolite (such as in vivo) of a compound of formula (I) may be beneficial by contributing to the biological activity observed of the compound of formula (I). Thus, in one embodiment, there is provided an active metabolite of the compound of formula (I) and its use as a pharmaceutical e.g. for the treatment or prevention of the diseases mentioned herein.

It is to be understood that the present invention encompasses all isomers of compounds of formula (I) including all geometric, tautomeric and optical forms, and mixtures thereof (e.g. racemic mixtures). Insofar as described herein, e.g., in claim 1, certain specific structural isomers are provided as part of the invention. In particular, the invention extends to all tautomeric forms of the compounds of formula (I). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exists as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹ and ¹³N, and would be useful in positron emission topography (PET) studies for examining substrate receptor occupancy.

In one embodiment, the compounds of formula (I) are provided in a natural isotopic form. In one embodiment, the compounds of formula (I) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I). In one embodiment, the atoms of the compounds of formula (I) are in an isotopic form which is not radioactive. In one embodiment, one or more atoms of the compounds of formula (I) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In another embodiment, a compound of formula (I) is provided whereby two or more atoms exist in an unnatural variant isotopic form. Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the purer forms used in the pharmaceutical compositions.

Therapeutic Indications

Compounds of formula (I) are of use in therapy, particularly for treating or preventing an inflammatory disease or a disease associated with an undesirable immune response. As shown in Biological Example 1 below, example compounds of formula (I) reduced cytokine release more effectively than dimethyl itaconate, as demonstrated by lower IC₅₀ values. Cytokines are important mediators of inflammation and immune-mediated disease as evidenced by the therapeutic benefit delivered by antibodies targeting them.

Thus, in a first aspect, the present invention provides a compound of formula (I) or a pharmaceutically acceptable salt and/or solvate thereof as defined herein, for use as a medicament. Also provided is a pharmaceutical composition comprising a compound of formula (I) or a pharmaceutically acceptable salt and/or solvate thereof as defined herein. Such a pharmaceutical composition contains the compound of formula (I) and a pharmaceutically acceptable carrier or excipient.

In a further aspect, the present invention provides a compound of formula (I) or a pharmaceutically acceptable salt and/or solvate thereof as defined herein, for use in treating or preventing an inflammatory disease or a disease associated with an undesirable immune response. In a further aspect, the present invention provides the use of a compound of formula (I) or a pharmaceutically acceptable salt and/or solvate thereof as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (I) or a pharmaceutically acceptable salt and/or solvate thereof as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof as defined herein, for use in treating an inflammatory disease or disease associated with an undesirable immune response. In one embodiment of the invention is provided the use of a compound of formula (I) or a pharmaceutically acceptable salt and/or solvate thereof as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in preventing an inflammatory disease or a disease associated with an undesirable immune response. In one embodiment of the invention is provided the use of a compound of formula (I) or a pharmaceutically acceptable salt and/or solvate thereof as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing an inflammatory disease. In one embodiment of the invention is provided the use of a compound of formula (I) or a pharmaceutically acceptable salt and/or solvate thereof as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing a disease associated with an undesirable immune response. In one embodiment of the invention is provided the use of a compound of formula (I) or a pharmaceutically acceptable salt and/or solvate thereof as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behget's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaffs brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis and chronic obstructive pulmonary disease.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

Administration

The compound of formula (I) is usually administered as a pharmaceutical composition. Thus, in 25 one embodiment, is provided a pharmaceutical composition comprising a compound of formula (I) and one or more pharmaceutically acceptable diluents or carriers.

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilize the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (-)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen(R), specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10 to 60% by weight, of the compound of formula (I), depending on the method of administration. The composition may contain from 0% to 99.9% by weight, for example 0% to 99% by weight, suitably 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), P13K inhibitors including pan-inhibitors or those targeting the p110δ and/or p110γ containing isoforms (e.g. idelalisib, copanlisib, duvelisib), interferon a receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4μ1 and/or α4μ7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1P inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) may display one or more of the following desirable properties:
low IC₅₀ values for inhibiting release of cytokines, e.g., IL-1β and/or IL-6, from cells;
low EC₅₀ and/or high Eₘₐₓ values for activating the NRF2 pathway;
high EC₅₀ and/or low Eₘₐₓ values for activating the NRF2 pathway;
enhanced efficacy through improved hydrolytic stability of carboxylic acid esters and/or augmented maximum response;
reduced dose and dosing frequency through improved pharmacokinetics;
improved oral systemic bioavailability;
reduced plasma clearance following intravenous dosing;
improved metabolic stability, e.g., as demonstrated by improved stability in plasma and/or hepatocytes;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation;
glutathione-sparing actions;
avoiding the oncometabolite fumaric acid (Kulkarni R. A. et al., 2019).

### Abbreviations

Ac acetyl
BBFO broadband fluorine observe
BEH ethylene bridged hybrid
n-Bu n-butyl
Cbz carboxybenzyl, i.e., benzyl-O—C(═O)—
CSH charged surface hybrid
d doublet
DAD diode array detector
DBU 1,8-diazabicyclo(5.4.0)undec-7-ene
DCC N,N′-dicyclohexylcarbodiimide
DCM dichloromethane
DIPEA N,N-diisopropylethylamine
DMAP 4-dimethylaminopyridine
DMF dimethyl fumarate
DMSO dimethyl sulfoxide
EDC 1-ethyl-3-(3-dimethylaminopropyl)carbodiimide
ee enantiomeric excess
Et ethyl
ES⁺ electrospray
FBS fetal bovine serum
g gram(s)
GSH glutathione
h(r) hour(s)
HPLC high-performance liquid chromatography
IL interleukin
IPA isopropyl alcohol
LDA lithium diisopropylamide
LCMS liquid chromatography-mass spectrometry
m multiplet
M molar concentration/molar mass
m/z mass to charge ratio
mCPBA meta-chloroperoxybenzoic acid
Me methyl
(M)Hz (mega)hertz
mg milligram
min(s) minute(s)
mL millilitres
mmol millimole
MS mass spectrometry
MTBE methyl tertiary-butyl ether
nm nanometer
NMR nuclear magnetic resonance
NMP N-methyl-2-pyrrolidone
PBS phosphate buffered saline
PDA photodiode array
Ph phenyl
i-Pr iso-propyl
rpm revolutions per minute
RT room temperature
s singlet
t triplet
TBS tert-butyl silyl
TEA triethylamine
Tf trifluoromethanesulfonyl, i.e., CF₃SO₂—
TFA trifluoroacetic acid
THF tetrahydrofuran
μL microlitre
μM micromolar
UPLC ultra performance liquid chromatography
wt. weight
° C. degrees centigrade

### EXAMPLES

Analytical Equipment

Thin layer chromatography (TLC) was performed on silica gel plates (GF254, glass, silica gel size: 400˜600 mesh). Spots were visualized by UV light (214 and 254 nm) or color reagents (iodine, KMnO₄ aq.).

All NM R spectra were recorded on a Bruker 400 (400 MHz) spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration.

The following analytical LCMS equipment and methods were used:

LCMS/HPLC Instrument Details

| System | Instrument Name | LC Detector | ELS detector | Mass detector |
|---|---|---|---|---|
| 2 | Agilent LCMS 1200 | G1315C DAD | 380 ELSD | Agilent G6110A |

LCMS/HPLC Method Details

| | | | | | | Column | Flow |
|---|---|---|---|---|---|---|---|
| Method | Solvent | | | UV | Mass | Temp. | Rate |
| Name | System | Column | Gradient | range | Range | ° C. | ml/min |
| B | A) water + | Waters X- | From 95:5 to | 190- | 100- | 40 | 2.0 |
| | 0.05% TFA | Bridge C18 | 0:100 in 1.6 | 400 | 1100 | | |
| | B) | (50 mm × | min, 0:100 for | nm | amu | | |
| | acetonitrile + | 4.6 mm × | 1.4 min, from | | | | |
| | 0.05% TFA | 3.5 μm) | 0:100 to 95:5 in | | | | |
| | | | 0.05 min, 95:5 | | | | |
| | | | for 0.7 min | | | | |
| C | A) water + | Halo C18 | From 95:5 to | 190- | 100- | 40 | 3.0 |
| | 0.05% TFA | (30 mm × | 0:100 in 0.8 | 400 | 1100 | | |
| | B) | 4.6 mm × | min, 0:100 for | nm | amu | | |
| | acetonitrile + | 2.7 μm) | 0.4 min, from | | | | |
| | 0.05% TFA | | 0:100 to 95:5 in | | | | |
| | | | 0.01 min, 95:5 | | | | |
| | | | for 0.2 min | | | | |

Commercial Materials

All starting materials are commercially available. Dimethyl itaconate was purchased from Sigma-Aldrich (product number: 109533); dimethyl fumarate and 1-bromo-4-(trifluoromethyl)benzene are commercially available for example from Sigma-Aldrich.

### Intermediate 1—3-((2,2,2-trichloroethoxy)carbonyl)but-3-enoic acid

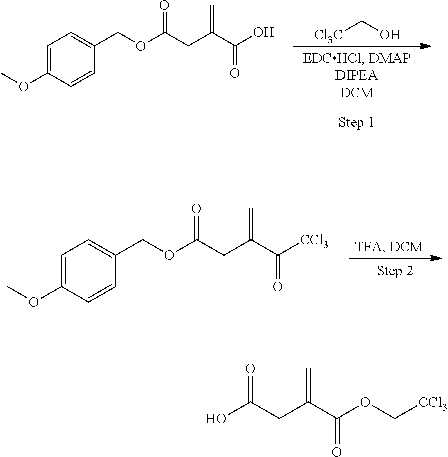

### Step 1

To a solution of 4-((4-methoxybenzyl)oxy)-2-methylene-4-oxobutanoic acid (30.0 g, 120 mmol), 2,2,2-trichloroethan-1-ol (19.7 g, 132 mmol), DMAP (11.7 g, 96 mmol) and DIPEA (46.4 g, 360 mmol) in DCM (500 mL) at 0° C. was added EDC·HCl (34.6 g, 180 mmol), and the resulting pale-yellow mixture was stirred at room temperature overnight. The mixture was quenched with dilute aqueous HCl (0.5 M), the phases were separated and the aqueous layer was extracted with DCM (3×500 mL). The combined organic phases were washed with brine, dried over Na₂SO₄ and filtered. The filtrate was concentrated under reduced pressure at 30° C., and the residue was purified by flash column chromatography (120 g silica, 0-20% MTBE/petroleum ether) to give 4-(4-methoxybenzyl) 1-(2,2,2-trichloroethyl) 2-methylenesuccinate (35 g, 91.7 mmol, 76%) as a colorless oil. LCMS: (System 2, Method C) m/z 402.8/404.8 (M+Na)⁺ (ES⁺).

### Step 2

A solution of 4-(4-methoxybenzyl) 1-(2,2,2-trichloroethyl) 2-methylenesuccinate (35.0 g, 91.7 mmol) in TFA (40 mL) and DCM (80 mL) was stirred at room temperature for 16 h. The mixture was concentrated under reduced pressure at 30° C. and the residue was purified by reversed phase column chromatography (330 g C18 silica; flow rate: 60 mL/min; 60-80% MeCN/10 mM formic acid/water; collection wavelength: 214 nm). The collected fractions were concentrated under reduced pressure at 30° C. to remove MeCN, and the residue was lyophilized to give 3-((2,2,2-trichloroethoxy)carbonyl)but-3-enoic acid (23.0 g, 88.0 mmol, 96%) as a colorless oil. LCMS: (System 2, Method C) m/z 282.8/284.8 (M+Na)⁺ (ES⁺).

### Intermediate 2—1-(4-((trifluoromethyl)thio)phenyl)cyclobutan-1-ol

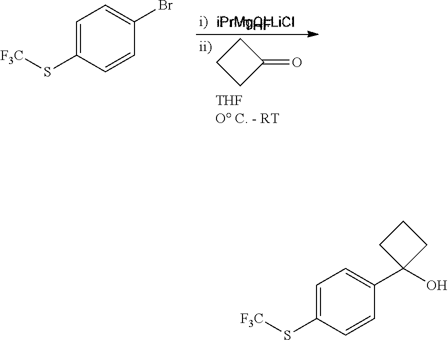 [SMILES: FC(F)(F)Sc1ccc(Br)cc1 ; O=C1CCC1 ; OC1(c2ccc(SC(F)(F)F)cc2)CCC1]

To a solution of (4-bromophenyl)(trifluoromethyl)sulfane (7.75 g, 30 mmol) in THF (75 mL) at room temperature was added a solution of iPrMgCl·LiCI in hexane (1.3 M, 23 ml, 30 mmol) and the mixture was stirred at room temperature for 1 h. The solution was then cooled to 0° C., cyclobutanone (1.62 g, 23.2 mmol) was added, and the mixture was stirred at room temperature for 3 h. The reaction mixture was quenched with saturated aqueous ammonium chloride solution (50 mL), the phases were separated, and the aqueous layer was extracted with MTBE (2×50 mL). The combined organic layers were washed with brine, dried over Na₂SO₄, filtered and concentrated under reduced pressure at 30° C. The residue was purified by flash column chromatography (80 g silica, 0-14% MTBE/petroleum ether) to give 1-(4-((trifluoromethyl)thio)phenyl)cyclobutan-1-ol (2.5 g, 10.1 mmol, 33%) as a yellow oil. ¹H NMR (400 MHz, CDCl₃) δ: 7.69-7.62 (m, 2H), 7.59-7.53 (m, 2H), 2.61-2.50 (m, 2H), 2.45-2.33 (m, 2H), 2.17-2.00 (m, 1H), 1.83-1.69 (m, 1H). One exchangeable proton not observed.

### Intermediate 3—1-(3-chloro-4-(trifluoromethyl)phenyl)cyclobutan-1-ol

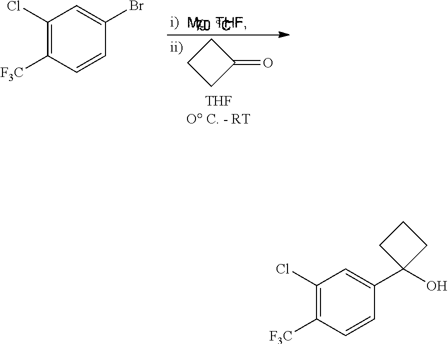 [SMILES: FC(F)(F)c1ccc(Br)cc1Cl ; O=C1CCC1 ; OC1(c2ccc(C(F)(F)F)c(Cl)c2)CCC1]

To a suspension of Mg metal (374 mg, 15.6 mmol) in THF (50 mL) at 70° C. was added 4-bromo-2-chloro-1-(trifluoromethyl)benzene (4 g, 15.6 mmol) and the mixture was stirred at 70° C. for 1 h. The solution was cooled to O ° C., cyclobutanone (1.09 g, 15.6 mmol) was added, and the mixture was stirred at room temperature for 3 h. The reaction mixture was quenched with saturated aqueous ammonium chloride solution (50 mL), the phases were separated, and the aqueous layer was extracted with MTBE (2×50 mL). The combined organic layers were washed with brine, dried over Na₂SO₄, filtered and concentrated under reduced pressure at 30° C. The residue was purified by flash column chromatography (80 g silica, 0-14% MTBE/petroleum ether) to give 1-(3-chloro-4-(trifluoromethyl)phenyl)cyclobutan-1-ol (2.6 g, 10.4 mmol, 69%) as a yellow oil. ¹H NMR (400 MHz, CDCl₃) δ: 7.68 (d, J=8.2 Hz, 1H), 7.66 (s, 1H), 7.52-7.47 (m, 1H), 2.59-2.48 (m, 2H), 2.45-2.34 (m, 2H), 2.16-2.03 (m, 1H), 1.85-1.72 (m, 1H). One exchangeable proton not observed.

### Intermediate 4—1-(3-fluoro-4-(trifluoromethyl)phenyl)cyclobutan-1-ol

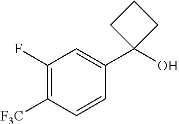 [SMILES: OC1(c2ccc(C(F)(F)F)c(F)c2)CCC1]

Prepared by an analogous method to Intermediate 3 starting from 4-bromo-2-fluoro-1-(trifluoromethyl)benzene (7.0 g, 28.8 mmol). Yield 3.65 g, 15.0 mmol, 54%. Yellow oil. ¹H NMR (400 MHz, CDCl₃) δ: 7.60 (t, J=7.8 Hz, 1H), 7.41-7.33 (m, 2H), 2.59-2.48 (m, 2H), 2.46-2.34 (m, 2H), 2.16-2.02 (m, 2H), 1.86-1.71 (m, 1H).

### Intermediate 5—1-(4-(pentafluoro-λ⁶-sulfaneyl)phenyl)cyclobutan-1-ol

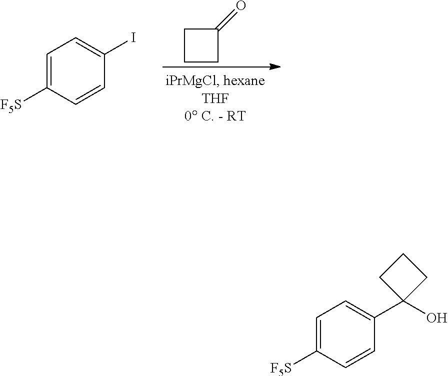

To a solution of pentafluoro(4-iodophenyl)-λ⁶-sulfane (2.36 g, 7.1 mmol) in THF (23 mL) at room temperature was added iPrMgCl solution in hexane (2 M, 3.6 ml, 7.2 mmol) and the mixture was stirred at room temperature for 1 h. After the solution was cooled to 0° C., cyclobutanone (0.497 g, 7.1 mmol) was added, and the mixture was stirred at room temperature for 3 h. The reaction mixture was quenched with saturated aqueous ammonium chloride solution (30 mL), the phases were separated and the aqueous layer was extracted with MTBE (2×30 mL). The combined organic layers were washed with brine, dried over Na₂SO₄, filtered and concentrated under reduced pressure at 30° C. The residue was purified by flash column chromatography (40 g silica, 0-14% MTBE/petroleum ether) to give 1-(4-(pentafluoro-λ⁶-sulfaneyl)phenyl)cyclobutan-1-ol (1.9 g, 6.9 mmol, 95%) as a yellow oil. ¹H NMR (400 MHz, CDCl₃) δ: 7.78-7.73 (m, 2H), 7.61 (d, J=9.0 Hz, 2H), 2.61-2.50 (m, 2H), 2.46-2.34 (m, 2H), 2.15-2.01 (m, 2H), 1.84-1.69 (m, 1H).

### Intermediate 6—3-(4-(trifluoromethyl)phenyl)oxetan-3-ol

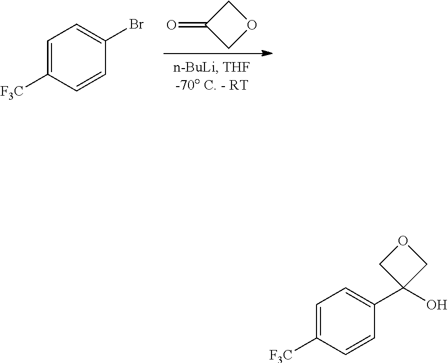

To the solution of 1-bromo-4-(trifluoromethyl)benzene (1.25 g, 5.56 mmol) in THF (15 mL) at −70° C. was added n-BuLi solution in hexane (2.5 M, 2.2 mL, 5.56 mmol) and the mixture was stirred at −70° C. for 1 h. Oxetan-3-one (400 mg, 5.56 mmol) was then added at −70° C., and the mixture was stirred at room temperature for 1 h. The reaction mixture was quenched with dilute aqueous HCl (0.5 M, 10 mL), the phases were separated and the aqueous layer was extracted with MTBE (2×20 mL). The combined organic layers were washed with brine, dried over Na₂SO₄, filtered and concentrated under reduced pressure at 30° C., and the residue was purified by flash column chromatography (20 g silica, 0-24% MTBE/petroleum ether) to give 3-(4-(trifluoromethyl)phenyl)oxetan-3-ol (700 mg, 3.21 mmol, 58%) as a yellow oil. ¹H NMR (400 MHz, CDCl₃) δ: 7.78 (d, J=8.1 Hz, 2H), 7.69 (d, J=8.0 Hz, 2H), 4.95 (d, J=7.6 Hz, 2H), 4.88 (d, J=7.6 Hz, 2H), 2.63 (s, 1H).

### Intermediate 7—4-(1-hydroxycyclobutyl)benzonitrile

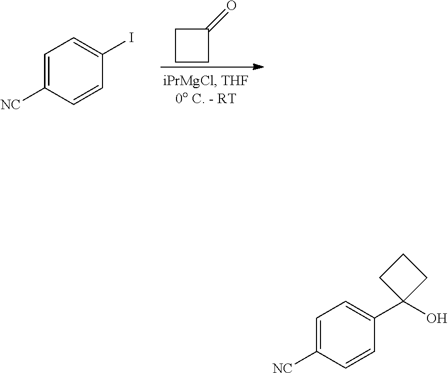

To a solution of 4-iodobenzonitrile (5 g, 22 mmol) in THF (70 mL) at 0° C. was added iPrMgCl solution in THF (1.3 M, 17 ml, 22 mmol) and the mixture was stirred at room temperature for 1 h. After the solution was cooled to 0° C., cyclobutanone (1.54 g, 22 mmol) was added, and the mixture was stirred at room temperature for 3 h. The reaction mixture was quenched with saturated aqueous ammonium chloride solution (50 mL), the phases were separated and the aqueous layer was extracted with MTBE (2×30 mL). The combined organic layers were washed with brine, dried over Na₂SO₄, filtered and concentrated under reduced pressure at 30° C. The residue was purified by flash column chromatography (40 g silica, 0-14% MTBE/petroleum ether) to give 4-(1-hydroxycyclobutyl)benzonitrile (3.5 g, 20.2 mmol, 92%) as a yellow oil. ¹H NMR (400 MHz, CDCl3) δ: 7.67-7.58 (m, 4H), 2.57-2.47 (m, 2H), 2.45-2.33 (m, 2H), 2.14-2.01 (m, 1H), 1.83-1.69 (m, 1H). One exchangeable proton not observed.

### Intermediate 8—1-(4′-(trifluoromethyl)-[1,1′-biphenyl]-4-yl)cyclobutan-1-ol

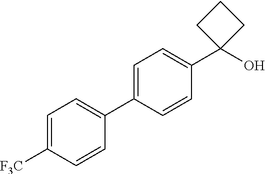 [SMILES: OC1(c2ccc(-c3ccc(C(F)(F)F)cc3)cc2)CCC1]

Prepared by an analogous method to Intermediate 6 starting from 4-bromo-4′-(trifluoromethyl)-1,1′-biphenyl (1.50 g, 4.98 mmol) and cyclobutanone (350 mg, 5.0 mmol). Yield 1.3 g, 4.45 mmol, 89%. Yellow oil. ¹H NMR (400 MHz, CDCl₃) δ: 7.70 (s, 4H), 7.62 (s, 4H), 2.67-2.56 (m, 2H), 2.48-2.36 (m, 2H), 2.13-2.00 (m, 2H), 1.82-1.68 (m, 1H).

### Intermediate 9—1-(5-(trifluoromethyl)pyridin-2-yl)cyclobutan-1-ol

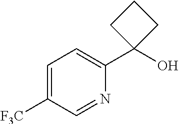 [SMILES: OC1(c2ccc(C(F)(F)F)cn2)CCC1]

Prepared by an analogous method to Intermediate 8 starting from 2-bromo-5-(trifluoromethyl)pyridine (400 mg, 1.78 mmol) except that toluene was used as solvent in place of THF. Yield 250 mg, 1.15 mmol, 65%. Pale-yellow oil. ¹H NMR (400 MHz, CDCl₃) δ: 8.80 (s, 1H), 7.99 (dd, J=8.3, 2.3 Hz, 1H), 7.72 (d, J=8.2 Hz, 1H), 4.72 (s, 1H), 2.63-2.46 (m, 4H), 2.19-2.05 (m, 1H), 1.99-1.83 (m, 1H).

### Intermediate 10—1-(4-(difluoro(phenyl)methyl)phenyl)cyclobutan-1-ol

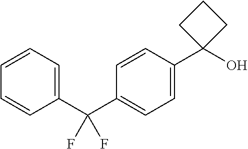 [SMILES: OC1(c2ccc(C(F)(F)c3ccccc3)cc2)CCC1]

Prepared by an analogous method to Intermediate 8 starting from 1-bromo-4-(difluoro(phenyl)methyl)benzene (4.5 g, 15.9 mmol). Yield 2.2 g, 8.02 mmol, 51%. White solid. ¹H NMR (400 MHz, CDCl₃) δ: 7.59-7.47 (m, 6H), 7.46-7.38 (m, 3H), 2.61-2.50 (m, 2H), 2.43-2.32 (m, 2H), 2.11-1.93 (m, 2H). One exchangeable proton not observed.

### Intermediate 11—3,3-difluoro-1-(4-(trifluoromethyl)phenyl)cyclobutan-1-ol

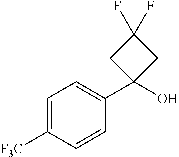 [SMILES: OC1(c2ccc(C(F)(F)F)cc2)CC(F)(F)C1]

Prepared by an analogous method to Intermediate 8 starting from 1-iodo-4-(trifluoromethyl)benzene (3.7 g, 13.7 mmol) and 3,3-difluorocyclobutan-1-one (1.44 g, 11.0 mmol). Yield 380 mg, 1.51 mmol, 12%. Yellow oil. ¹H NMR (400 MHz, CDCl3) δ: 7.71-7.57 (m, 4H), 3.23-2.97 (m, 4H). One exchangeable proton not observed.

### Intermediate 12—1-(4-(perfluoroethyl)phenyl)cyclobutan-1-ol

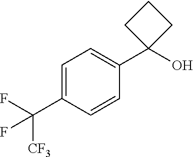 [SMILES: OC1(c2ccc(C(F)(F)C(F)(F)F)cc2)CCC1]

Prepared by an analogous method to Intermediate 2 starting from 1-bromo-4-(perfluoroethyl)benzene (3.0 g, 11.0 mmol) except that the iPrMgCl.LiCI was added at −30° C. before stirring at room temperature. Yield 200 mg, 0.75 mmol, 10%. Yellow oil. ¹H NMR (400 MHz, CDCl3) δ: 7.62 (q, J=8.4 Hz, 4H), 2.62-2.51 (m, 2H), 2.46-2.33 (m, 2H), 2.17-2.01 (m, 1H), 1.84-1.69 (m, 1H). One exchangeable proton not observed.

### Intermediate 13—1-(5-(trifluoromethyl)thiophen-2-yl)cyclobutan-1-ol

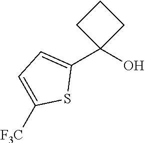 [SMILES: OC1(c2ccc(C(F)(F)F)s2)CCC1]

Prepared by an analogous method to Intermediate 8 starting from 2-bromo-5-(trifluoromethyl)thiophene (300 mg, 1.3 mmol). Yield 200 mg, 0.9 mmol, 69%. Yellow oil. ¹H NMR (400 MHz, CDCl3) δ: 7.33-7.29 (m, 1H), 7.03-6.99 (m, 1H), 2.59-2.39 (m, 4H), 2.07-1.94 (m, 1H), 1.88-1.72 (m, 1H). One exchangeable proton not observed.

### Intermediate 14—(S)-2,2,2-trifluoro-1-(4-(trifluoromethyl)phenyl)ethan-1-ol and Intermediate 15—(R)-2,2,2-trifluoro-1-(4-(trifluoromethyl)phenyl)ethan-1-ol

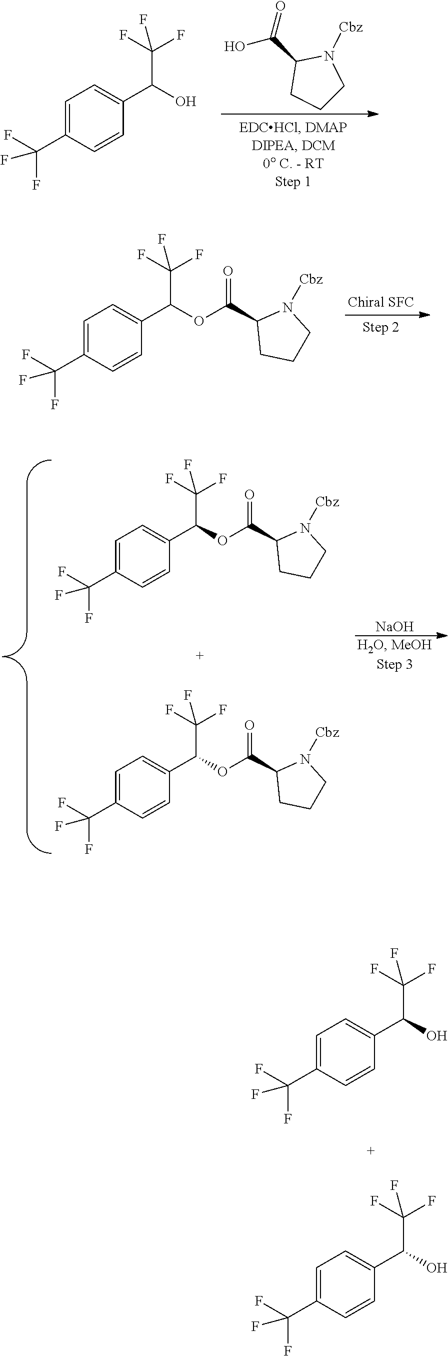

### Step 1

To a solution of 2,2,2-trifluoro-1-(4-(trifluoromethyl)phenyl)ethan-1-ol (3.0 g, 12.3 mmol), ((benzyloxy)carbonyl)-L-proline (3.06 g, 12.3 mmol), DMAP (1.80 g, 14.8 mmol) and DIPEA (6.35 g, 49.2 mmol) in DCM (50 mL) at 0° C. was added EDC·HCl (4.72 g, 24.6 mmol), and the resulting pale yellow mixture was stirred at room temperature for 2 h. The mixture was quenched with dilute aqueous HCl (0.5 M, 40 mL), the phases were separated, and the aqueous phase was extracted with DCM (2×50 mL). The combined organic phases were washed with brine, dried over Na₂SO₄ and filtered. The filtrate was concentrated under reduced pressure at 40° C., and the residue was purified by flash column chromatography (40 g silica, 0-12% MTBE/petroleum ether) to give 1-benzyl 2-(2,2,2-trifluoro-1-(4-(trifluoromethyl)phenyl)ethyl) (2S)-pyrrolidine-1,2-dicarboxylate (4.0 g, 8.41 mmol, 68%) as a pale yellow oil. LCMS (System 2, Method B) m/z 475.9 (M+H)⁺ (ES⁺).

### Step 2

1-Benzyl 2-(2,2,2-trifluoro-1-(4-(trifluoromethyl)phenyl)ethyl) (2S)-pyrrolidine-1,2-dicarboxylate (1.90 g, 4.00 mmol) was separated into individual diastereomers using chiral SFC (Column: CHIRALPAK AD-5 5 μm 30×250 mm; Column temperature: 35° C.; Flow rate: 45 mL/min; Solvent system: 20% IPA/CO₂; Collection wavelength: 215 nm). The collected fractions were concentrated under reduced pressure at 40° C. to remove IPA to give 1-benzyl 2-((S)-2,2,2-trifluoro-1-(4-(trifluoromethyl)phenyl)ethyl) (S)-pyrrolidine-1,2-dicarboxylate (900 mg, 1.89 mmol, 47%) as the first eluting peak and 1-benzyl 2-((R)-2,2,2-trifluoro-1-(4-(trifluoromethyl)phenyl)ethyl) (S)-pyrrolidine-1,2-dicarboxylate (950 mg, 2.00 mmol, 50%) as the second eluting peak. Chiral SFC analysis (Column: CHIRALPAKAD-3 3 μm 4.6×100 mm; Column temperature: 35° C.; Flow rate: 2 mL/min; Solvent system: 15% IPA/CO₂; Collection wavelength: 200-400 nm): 1-benzyl 2-((S)-2,2,2-trifluoro-1-(4-(trifluoromethyl) phenyl)ethyl) (S)-pyrrolidine-1,2-dicarboxylate Rt=0.887 min, 100% ee; 1-benzyl 2-((R)-2,2,2-trifluoro-1-(4-(trifluoromethyl)phenyl)ethyl) (S)-pyrrolidine-1,2-dicarboxylate Rt=1.089 min, 97.9% ee.

### Step 3

To a solution of 1-benzyl 2-((S)-2,2,2-trifluoro-1-(4-(trifluoromethyl)phenyl)ethyl) (S)-pyrrolidine-1,2-dicarboxylate (900 mg, 1.89 mmol) in MeOH (6 mL) was added dilute aqueous NaOH solution (2 M, 2.84 mL, 5.68 mmol), and the reaction mixture was stirred at room temperature for 12 h. The mixture was concentrated under reduced pressure at 30° C. to give a residue, which was diluted with H₂O (5 ml). The phases were separated, and the aqueous phase was extracted with MTBE (2×5 ml). The combined organic layers were washed with brine, dried over Na₂SO₄, filtered and concentrated under reduced pressure at 30° C. to give (S)-2,2,2-trifluoro-1-(4-(trifluoromethyl)phenyl)ethan-1-ol (450 mg, 1.84 mmol, 97%) as a pale yellow oil. ¹H NMR (400 MHz, CDCl₃) δ: 7.69 (d, J=8.3 Hz, 2H), 7.63 (d, J=8.2 Hz, 2H), 5.17-5.07 (m, 1H), 2.83 (d, J=4.5 Hz, 1H). [α]_{D}²⁰=+21.67° (c=0.25 g/100 mL, EtOH).

By a similar procedure, starting from 1-benzyl 2-((R)-2,2,2-trifluoro-1-(4-(trifluoromethyl)phenyl)ethyl) (S)-pyrrolidine-1,2-dicarboxylate (950 mg, 2.00 mmol), (R)-2,2,2-trifluoro-1-(4-(trifluoromethyl)phenyl)ethan-1-ol (480 mg, 1.97 mmol, 98%) was obtained as a pale yellow oil. ¹H NMR (400 MHz, CDCl₃) δ: 7.69 (d, J=8.3 Hz, 2H), 7.63 (d, J=8.2 Hz, 2H), 5.17-5.06 (m, 1H), 2.84 (d, J=4.1 Hz, 1H). [α]_{D}²⁰=−20.00° (c=0.25 g/100 mL, EtOH).

### Intermediate 16—2-(4-(trifluoromethyl)phenyl)spiro[3.3]heptan-2-ol

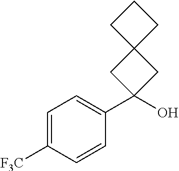 [SMILES: OC1(c2ccc(C(F)(F)F)cc2)CC2(CCC2)C1]

Prepared by an analogous method to Intermediate 8 starting from 1-bromo-4-(trifluoromethyl)benzene (440 mg, 1.96 mmol) and spiro[3.3]heptan-2-one (237 mg, 2.16 mmol). Yield 300 mg, 1.17 mmol, 60%. Pale yellow oil. ¹H NMR (400 MHz, CDCl₃) δ: 7.61 (d, J=8.3 Hz, 2H), 7.56 (d, J=8.3 Hz, 2H), 2.67-2.60 (m, 2H), 2.46-2.39 (m, 2H), 2.19 (t, J=7.3 Hz, 2H), 1.98-1.90 (m, 2H), 1.90-1.80 (m, 2H). One exchangeable proton not observed.

### Intermediate 17—1-(4-(3,3,3-trifluoroprop-1-yn-1-yl)phenyl)cyclobutan-1-ol

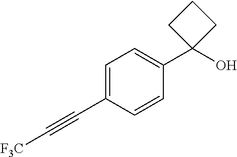 [SMILES: OC1(c2ccc(C#CC(F)(F)F)cc2)CCC1]

Prepared by an analogous method to Intermediate 8 starting from 1-bromo-4-(3,3,3-trifluoroprop-1-yn-1-yl)benzene (500 mg, 2.01 mmol). Yield 140 mg, 0.58 mmol, 29%. Colorless oil. ¹H NMR (400 MHz, CDCl₃) δ: 7.60-7.51 (m, 4H), 2.60-2.48 (m, 2H), 2.45-2.32 (m, 2H), 2.15.2.00 (m, 2H), 1.82-1.67 (m, 1H).

### Intermediate 18—1-(4-(trifluoromethyl)phenyl)prop-2-yn-1-ol Enantiomer 1 and

### Intermediate 19—1-(4-(trifluoromethyl)phenyl)prop-2-yn-1-ol Enantiomer 2

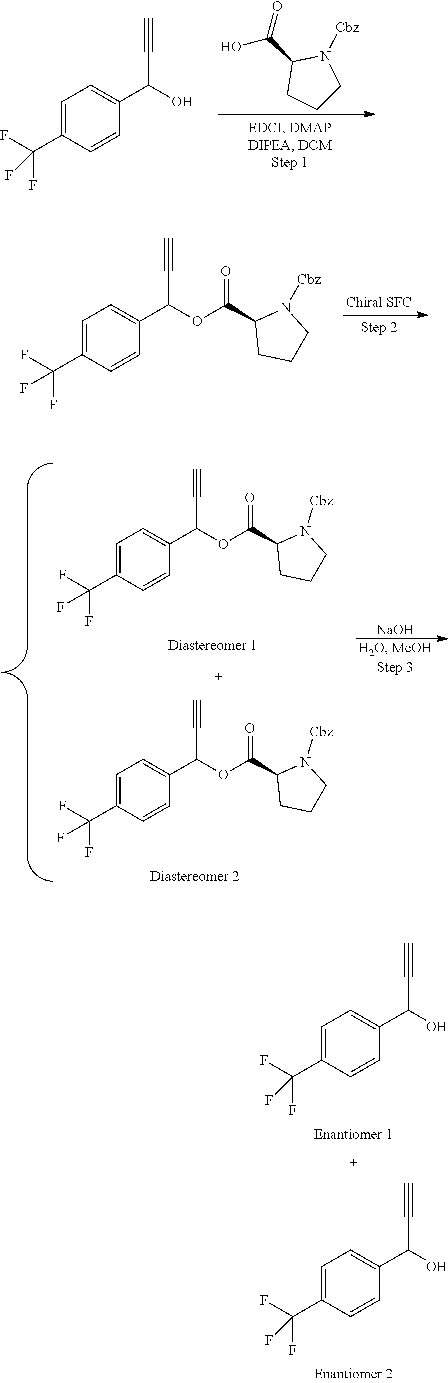

### Step 1

To a solution of 1-(4-(trifluoromethyl)phenyl)prop-2-yn-1-ol (2.0 g, 10.0 mmol), ((benzyloxy)carbonyl)-L-proline (3.0 g, 12.0 mmol), DMAP (1.46 g, 12.0 mmol) and DIPEA (5.16 g, 40.0 mmol) in DCM (30 mL) at 0° C. was added EDC·HCl (3.84 g, 20.0 mmol), and the resulting pale yellow mixture was stirred at room temperature for 2 h. The mixture was quenched with dilute aqueous HCl (0.5 M, 30 mL), the phases were separated, and the aqueous phase was extracted with DCM (2×30 mL). The combined organic phases were washed with brine, dried over Na₂SO₄ and filtered. The filtrate was concentrated under reduced pressure at 35° C., and the residue was purified by flash column chromatography (40 g silica, 0-10% MTBE/petroleum ether) to give 1-benzyl 2-(1-(4-(trifluoromethyl)phenyl)prop-2-yn-1-yl) (2S)-pyrrolidine-1,2-dicarboxylate (3.0 g, 6.95 mmol, 70%) as a pale yellow oil. LCMS (System 2, Method B) m/z 453.9 (M+H)⁺ (ES⁺).

### Step 2

1-Benzyl 2-(1-(4-(trifluoromethyl)phenyl)prop-2-yn-1-yl) (2S)-pyrrolidine-1,2-dicarboxylate (1.90 g, 4.41 mmol) was separated into individual diastereomers using chiral SFC (Column: CHIRALCEL OJ-5 5 μm 30×250 mm; Column temperature: 35° C.; Flow rate: 45 mL/min; Solvent system: 20% MeOH/CO₂; Collection wavelength: 215 nm). The collected fractions were concentrated under reduced pressure at 40° C. to remove MeOH to give 1-benzyl 2-(1-(4-(trifluoromethyl)phenyl)prop-2-yn-1-yl) (2S)-pyrrolidine-1,2-dicarboxylate Diastereomer 1 (880 mg, 2.04 mmol, 46%) as the first eluting peak and 1-benzyl 2-(1-(4-(trifluoromethyl)phenyl)prop-2-yn-1-yl) (2S)-pyrrolidine-1,2-dicarboxylate Diastereomer 2 (930 mg, 2.16 mmol, 49%) as the second eluting peak. Chiral SFC analysis (Column: CHIRALCEL OJ-3 3 μm 4.6×100 mm; Column temperature: 35° C.; Flow rate: 2 mL/min; Solvent system: 15% MeOH/CO₂; Collection wavelength: 200-400 nm): 1-benzyl 2-(1-(4-(trifluoromethyl)phenyl)prop-2-yn-1-yl) (2S)-pyrrolidine-1,2-dicarboxylate Diastereomer 1 Rt=0.929 min, 100% ee; 1-benzyl 2-(1-(4-(trifluoromethyl)phenyl)prop-2-yn-1-yl) (2S)-pyrrolidine-1,2-dicarboxylate Diastereomer 2 Rt=1.050 min, 95.4% ee.

### Step 3

To a solution of 1-benzyl 2-(1-(4-(trifluoromethyl)phenyl)prop-2-yn-1-yl) (2S)-pyrrolidine-1,2-dicarboxylate Diastereomer 1 (500 mg, 1.16 mmol) in MeOH (5 mL) was added dilute aqueous NaOH solution (2 M, 1.74 mL, 3.48 mmol), and the reaction mixture was stirred at room temperature for 4 h. The mixture was concentrated under reduced pressure at 30° C. to give a residue, which was diluted with H₂O (5 ml). The phases were separated, and the aqueous phase was extracted with MTBE (2×5 ml). The combined organic layers were washed with brine, dried over Na₂SO₄, filtered and concentrated under reduced pressure at 30° C. to give 1-(4-(trifluoromethyl)phenyl)prop-2-yn-1-ol Enantiomer 1 (230 mg, 1.15 mmol, 99%) as a pale yellow oil. ¹H NMR (400 MHz, CDCl₃) δ: 7.72-7.63 (m, 4H), 5.54 (d, J=4.5 Hz, 1H), 2.70 (d, J=2.3 Hz, 1H), 2.32 (d, J=6.0 Hz, 1H).

By a similar procedure, starting from 1-benzyl 2-(1-(4-(trifluoromethyl)phenyl)prop-2-yn-1-yl) (2S)-pyrrolidine-1,2-dicarboxylate Diastereomer 2 (500 mg, 1.16 mmol), 1-(4-(trifluoromethyl)phenyl)prop-2-yn-1-ol Enantiomer 2 (220 mg, 1.10 mmol, 95%) was obtained as a pale yellow oil. ¹H NMR (400 MHz, CDCl₃) δ: 7.71-7.63 (m, 4H), 5.54 (d, J=5.1 Hz, 1H), 2.71 (d, J=2.3 Hz, 1H), 2.32 (d, J=5.8 Hz, 1H).

### Intermediate 20—3-(4-(trifluoromethyl)phenyl)thietan-3-ol

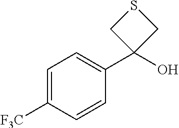 [SMILES: OC1(c2ccc(C(F)(F)F)cc2)CSC1]

Prepared by an analogous method to Intermediate 6 starting from 1-iodo-4-(trifluoromethyl)benzene (2.00 g, 7.35 mmol) and thietan-3-one (648 mg, 7.35 mmol). Yield 1.20 g, 5.12 mmol, 70%. Colorless oil. ¹H NMR (400 MHz, CDCl₃) δ: 7.85 (d, J=8.0 Hz, 2H), 7.68 (d, J=8.1 Hz, 2H), 3.66-3.58 (m, 4H), 2.85 (s, 1H).

### Intermediate 21—(cis)-3-hydroxy-3-(4-(trifluoromethyl) phenyl)cyclobutane-1-carbonitrile

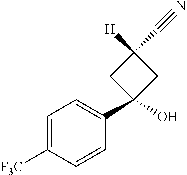 [SMILES: N#C[C@H]1C[C@](O)(c2ccc(C(F)(F)F)cc2)C1]

Prepared by an analogous method to Intermediate 6 starting from 1-iodo-4-(trifluoromethyl)benzene (2.00 g, 7.35 mmol) and 3-oxocyclobutane-1-carbonitrile (700 mg, 7.35 mmol). Yield 1.26 g, 5.22 mmol, 71%. Colorless oil. ¹H NMR (400 MHz, CDCl₃) δ: 7.67 (d, J=8.2 Hz, 2H), 7.58 (d, J=7.9 Hz, 2H), 3.08-2.95 (m, 2H), 2.90-2.76 (m, 3H), 2.58 (s, 1H).

### Intermediate 22—(cis)-3-methoxy-1-(4-(trifluoromethyl)phenyl)cyclobutan-1-ol

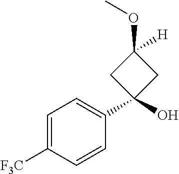 [SMILES: CO[C@H]1C[C@](O)(c2ccc(C(F)(F)F)cc2)C1]

Prepared by an analogous method to Intermediate 6 starting from 1-iodo-4-(trifluoromethyl)benzene (1.0 g, 3.68 mmol) and 3-methoxycyclobutanone (368 mg, 3.68 mmol). Yield 600 mg, 2.4 mmol, 66%. Light yellow oil. ¹H NMR (400 MHz, CDCl₃) δ: 7.61 (m, 4H), 3.80-3.74 (m, 1H), 3.31 (s, 3H), 2.95-2.88 (m, 2H), 2.45-2.38 (m, 3H).

### Intermediate 23—3-(trifluoromethyl)-1-(4-(trifluoromethyl)phenyl)cyclobutanol

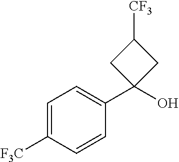 [SMILES: OC1(c2ccc(C(F)(F)F)cc2)CC(C(F)(F)F)C1]

Prepared by an analogous method to Intermediate 6 starting from 1-iodo-4-(trifluoromethyl)benzene (500 mg, 1.84 mmol) and 3-(trifluoromethyl)cyclobutanone (254 mg, 1.84 mmol). Yield 300 mg, 65% as mixture of cis and trans stereoisomers in 20:1 ratio. Yellow oil. ¹H NMR (400 MHz, CDCl₃) δ: 7.70-7-62 (m, 4H), 2.83-2.77 (m, 2H), 2.68-2.59 (m, 3H), 2.23 (s, 1H).

### Intermediate 24—3-methyl-1-(4-(trifluoromethyl)phenyl)cyclobutan-1-ol

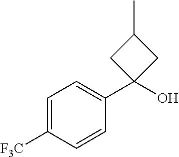 [SMILES: CC1CC(O)(c2ccc(C(F)(F)F)cc2)C1]

Prepared by an analogous method to Intermediate 6 starting from 1-iodo-4-(trifluoromethyl)benzene (1.94 g, 7.13 mmol) and 3-methylcyclobutan-1-one (600 mg, 7.13 mmol). Yield 1.10 g, 93% as mixture of cis and trans stereoisomers in 10:1 ratio. Colourless oil. ¹H NMR (400 MHz, CDCl₃) δ: 7.65-7.60 (m, 4H), 2.75-2.69 (m, 2H), 2.10 (s, 1H), 2.08-1.99 (m, 3H), 1.20 (d, J=6.0 Hz, 3H).

### Intermediate 25—(cis)-3-methyl-1-(4-(trifluoromethyl)phenyl)cyclobutan-1-ol

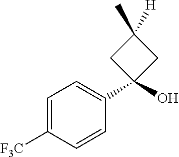 [SMILES: C[C@H]1C[C@](O)(c2ccc(C(F)(F)F)cc2)C1]

3-methyl-1-(4-(trifluoromethyl)phenyl)cycloutan-1-ol(Intermediate 24, 700 mg, 3.04 mmol) was separated by SFC (Column: CHIRALPAK AD-5(30×250 mm 5 μm) (Daicel). Column temperature: 35° C. Flow Rate: CO₂ flow Rate: 36 mL/min; co solvent flow rate: 9 mL/min; total flow rate: 45 mL/min. Co solvent: methanol. Gradient: methanol 20%. Collection wavelength: 215 nm). The SFC fractions were concentrated under reduced pressure to remove methanol to give (cis)-3-methyl-1-(4-(trifluoromethyl)phenyl)cyclobutan-1-ol (500 mg, 100% de, 71%) and (trans)-3-methyl-1-(4-(trifluoromethyl)phenyl)cyclobutan-1-ol (30 mg, 100% de, 4%). Chiral HPLC: (Column: CHIRALPAK AD-3 (4.6×100 mm); Flow Rate: 2 mL/min; Co solvent: 15% methanol; collection wavelength: 200-400 nm) trans isomer Rt=0.898 min; cis isomer: Rt=1.039 min.

### Intermediate 26—3-(5-(trifluoromethyl)pyridin-2-yl)oxetan-3-ol

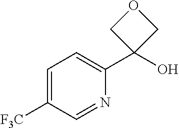 [SMILES: OC1(c2ccc(C(F)(F)F)cn2)COC1]

Prepared by an analogous method to Intermediate 6 starting from 2-bromo-5-(trifluoromethyl)pyridine (3.0 g, 13.30 mmol) and oxetan-3-one (1.05 g, 14.63 mmol). Yield 2.5 g, 85%. Light yellow oil. ¹H NMR (400 MHz, CDCl₃) δ: 8.82-8.79 (m, 1H), 8.17-8.12 (m, 2H), 5.64 (s, 1H), 5.12 (dd, J=5.2 Hz, 2H), 4.73 (dd, J=6 Hz, 2H).

### Intermediate 27—4-(4-(trifluoromethyl)phenyl)tetrahydro-2H-pyran-4-ol

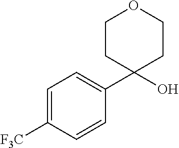 [SMILES: OC1(c2ccc(C(F)(F)F)cc2)CCOCC1]

Prepared by an analogous method to Intermediate 6 starting from 1-iodo-4-(trifluoromethyl)benzene (10 g, 7.35 mmol) and dihydro-2H-pyran-4(3H)-one (736 mg, 7.35 mmol). Yield 1.35 g, 74%. Light yellow oil. ¹H NMR (400 MHz, CDCl₃) δ: 7.62 (s, 4H), 3.97-3.89 (m, 4H), 2.22-2.15 (m, 2H), 1.68-1.65 (m, 3H).

### Intermediate 28—5-(4-(trifluoromethyl)phenyl)spiro[2.3]hexan-5-ol

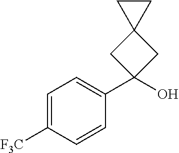 [SMILES: OC1(c2ccc(C(F)(F)F)cc2)CC2(CC2)C1]

Prepared by an analogous method to Intermediate 6 starting from 1-iodo-4-(trifluoromethyl)benzene (1.70 g, 6.25 mmol) and spiro[2.3]hexan-5-one (601 mg, 6.25 mmol). Yield 1.10 g, 73%. Colourless oil. ¹H NMR (400 MHz, CDCl₃) δ: 7.63 (d, J=8.0 Hz, 2H), 7.53 (d, J=8.4 Hz, 2H), 2.55-2.44 (m, 4H), 2.27 (s, 1H), 0.56-0.52 (m, 2H), 0.48-0.45 (m, 2H).

### Intermediate 29—1-methyl-3-(4-(trifluoromethyl)phenyl)azetidin-3-ol

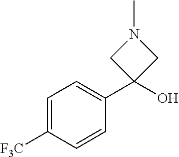 [SMILES: CN1CC(O)(c2ccc(C(F)(F)F)cc2)C1]

Prepared by an analogous method to Intermediate 6 starting from 1-iodo-4-(trifluoromethyl)benzene (1.70 g, 6.25 mmol), 1-methylazetidin-3-one hydrochloride (1.16 g, 9.56 mmol) and DIPEA (1.23 g, 9.56 mmol). Yield 200 mg, 9%. Colourless oil. LCMS (System 2, Method C) m/z 232.2 (M+H)⁺ (ES⁺).

### Intermediate 30—1-(5-bromopyridin-2-yl)cyclobutan-1-ol

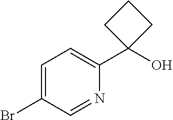 [SMILES: OC1(c2ccc(Br)cn2)CCC1]

Prepared by an analogous method to Intermediate 6 starting from 5-bromo-2-iodopyridine (5.0 g, 17.67 mmol) and cyclobutanone (1.24 g, 17.67 mmol). Yield 3.0 g, 75%. Light yellow oil. ¹H NMR (400 MHz, CDCl₃) δ: 8.58 (d, J=2 Hz, 1H), 7.86 (dd, J=8.4, 2.4 Hz, 1H), 7.49 (q, J=7.6 Hz, 1H), 4.67 (s, 1H), 2.57-2.45 (m, 4H), 2.12-2.04 (m, 1H), 1.92-1.82 (m, 1H).

### Intermediate 31—1-(5-chloropyridin-2-yl)cyclobutan-1-ol

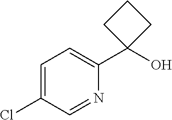 [SMILES: OC1(c2ccc(Cl)cn2)CCC1]

Prepared by an analogous method to Intermediate 6 starting from 2-bromo-5-chloropyridine (3.00 g, 15.6 mmol) and cyclobutanone (1.09 g, 15.6 mmol). Yield 2.40 g, 84%. Colourless oil. LCMS (System 2, Method C) m/z 184.2 (M+H)⁺ (ES⁺).

### Intermediate 32—1-(5-(trifluoromethyl)pyrimidin-2-yl)cyclobutan-1-ol

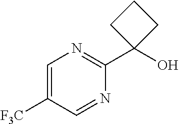 [SMILES: OC1(c2ncc(C(F)(F)F)cn2)CCC1]

Prepared by an analogous method to Intermediate 6 starting from 2-iodo-5-(trifluoromethyl)pyrimidine (2.50 g, 9.12 mmol) and cyclobutanone (639 mg, 9.12 mmol). Yield 140 mg, 7%. Light yellow oil. LCMS (System 2, Method C) m/z 219.2 (M+H)⁺ (ES⁺).

### Intermediate 33—1-(3,5-difluoro-4-(trifluoromethyl)phenyl)cyclobutan-1-ol

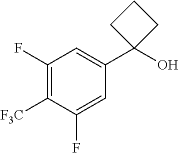 [SMILES: OC1(c2cc(F)c(C(F)(F)F)c(F)c2)CCC1]

Prepared by an analogous method to Intermediate 3 starting from 5-bromo-1,3-difluoro-2-(trifluoromethyl)benzene (6 g, 23.2 mmol) and cyclobutanone (1.62 g, 23.2 mmol). Yield 4 g, 69%. Light oil. ¹H NMR (400 MHz, CDCl₃) δ: 7.17 (d, J=10.8 Hz, 2H), 2.51-2.46 (m, 2H), 2.43-2.35 (m, 2H), 2.12-2.09 (m, 1H), 1.82-1.79 (m, 1H).

### Intermediate 34—1-(6-bromonaphthalen-2-yl)cyclobutan-1-ol

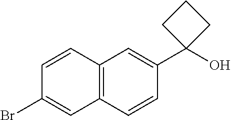 [SMILES: OC1(c2ccc3cc(Br)ccc3c2)CCC1]

Prepared by an analogous method to Intermediate 6 starting from, 6-dibromonaphthalene (1.0 g, 3.50 mmol) and cyclobutanone (246 mg, 3.50 mmol). Yield 600 mg, 62%. White solid. ¹H NMR (400 MHz, CDCl₃) δ: 7.99 (d, J=1.6 Hz, 1H), 7.88 (s, 1H), 7.78 (d, J=8.4 Hz, 1H), 7.72 (d, J=8.8 Hz, 1H), 7.66 (dd, J=8.8, 1.6 Hz, 1H), 7.56 (dd, J=8.8, 2.0 Hz, 1H), 2.72-2.62 (m, 2H), 2.48-2.40 (m, 2H), 2.10 (s, 1H), 2.13-2.03 (m, 1H), 1.81-1.70 (m, 1H).

### Intermediate 35—2-(5-(trifluoromethyl)pyridin-2-yl)propan-2-ol

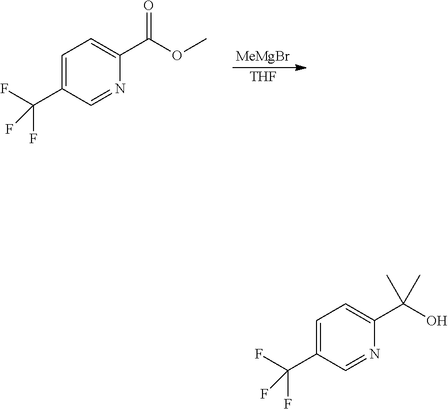

To the solution of methyl 5-(trifluoromethyl)picolinate (1.00 g, 7.31 mmol) in THF (15 mL) was added methyl magnesium bromide (6.1 mL, 18.27 mmol, 3.0M in diethyl ether) at 0° C.; and the mixture was stirred at 0° C. for 2 hrs. The reaction mixture was quenched with saturated NH₄Cl (10 mL), and the aqueous layer was extracted with MTBE (3×10 mL). The combined organic layers were washed by brine, dried over Na₂SO₄ and concentrated under reduced pressure. The residue was purified by flash column chromatography on silica (0-20% MTBE/petroleum ether) to give 2-(5-(trifluoromethyl)pyridin-2-yl)propan-2-ol (700 mg, 47% yield) as light-yellow oil. ¹H NMR (400 MHz, CDCl₃) δ: 8.80 (d, J=1.2 Hz, 1H), 7.94 (dd, J=8.4, 2.0 Hz, 1H), 7.55 (d, J=8.0 Hz, 1H), 4.46 (s, 1H), 1.58 (s, 6H).

### Intermediate 36—1-(5-(trifluoromethyl)pyridin-2-yl)cyclopropan-1-ol

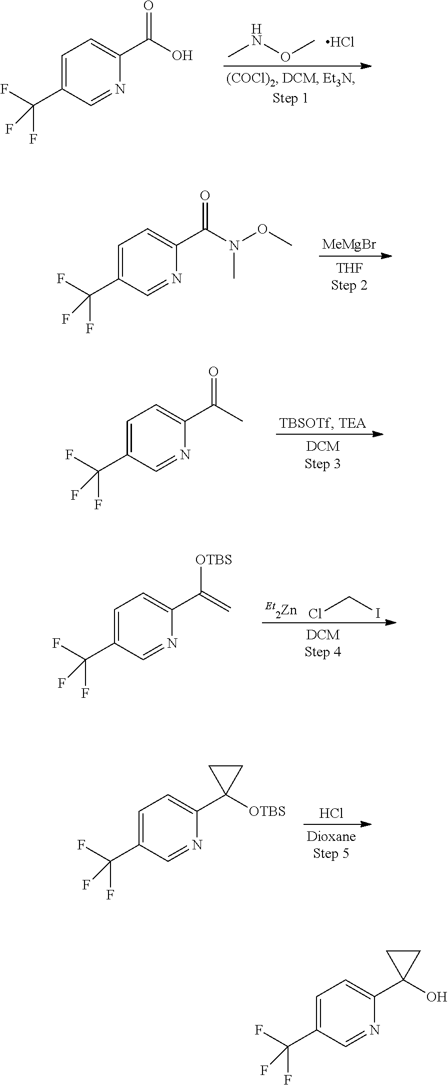

### Step 1

To a solution of 5-(trifluoromethyl)picolinic acid (4.5 g, 23.55 mmol) in DCM (100 mL) was added oxalyl dichloride (4.49 g, 35.33 mmol) at 0° C., and the reaction mixture was stirred at room temperature for 1 hr. The reaction mixture was concentrated at 30° C. under reduced pressure to give a yellow solid (4.5 g crude).

A solution of N,O-dimethylhydroxylamine hydrochloride (5.77 g, 58.87 mmol) and Et₃N (11.89 g, 117.75 mmol) in DCM (100 mL) was stirred at room temperature for 0.5 hr. To this solution was added the previously prepared yellow solid (4.5 g) at 0° C., and the mixture was allowed to stir at room temperature for 1 hr. The mixture was quenched with water (50 mL) and extracted with DCM (2×60 mL). The combined organic layers were washed with brine, dried over Na₂SO₄ concentrated under reduced pressure. The residue was purified by flash column chromatography on silica (0-20% MTBE/petroleum ether) to give N-methoxy-N-methyl-5-(trifluoromethyl)picolinamide (3.5 g, 63% yield) as yellow solid. LCMS (System 2, Method C) m/z 235.2 (M+H)⁺ (ES⁺).

### Step 2

To the solution of N-methoxy-N-methyl-5-(trifluoromethyl)picolinamide (3.5 g, 14.95 mmol) in THF (50 mL) was added MeMgBr (6.5 mL, 19.44 mmol, 3.0 M in 2-Methyl-THF) at 0° C.; and the mixture was stirred at rt for 2 hrs. The reaction mixture was quenched with aqueous saturated ammonium chloride (50 mL), and the aqueous layer was extracted with MTBE (2×50 mL). The combined organic layers were washed by brine, dried over Na₂SO₄ and concentrated under reduced pressure. The residue was purified by flash column chromatography on silica (0-10% MTBE/petroleum ether) to give 1-(5-(trifluoromethyl)pyridin-2-yl)ethanone (2.00 g, 70% yield) as yellow oil. LCMS (System 2, Method C) m/z 190.0 (M+H)⁺ (ES⁺).

### Step 3

To the solution of 1-(5-(trifluoromethyl)pyridin-2-yl)ethanone (1.00 g, 5.29 mmol) and Et₃N (1.60 g, 15.87 mmol) in DCM (20 mL) was added TBSOTf (1.75 g, 6.61 mmol) at 0° C.; and the mixture was stirred at rt for 16 hrs. The reaction mixture was quenched with water (20 mL) and the aqueous layer was extracted with DCM (2×20 mL). The combined organic layers were washed by brine, dried over Na₂SO₄, and concentrated under reduced pressure. The residue was purified by flash column chromatography on silica (0-10% MTBE/petroleum ether) to give 2-(1-(tert-butyldimethylsilyloxy)vinyl)-5-(trifluoromethyl)pyridine (1.40 g, 87% yield) as yellow oil. LCMS (System 2, Method C) m/z 304.1 (M+H)⁺ (ES⁺).

### Step 4

To the solution of Et₂Zn (15.21 mL, 15.21 mmol, 1.0 M solution in hexanes) in DCM (30 mL) was added chloroiodomethane (5.42 g, 30.73 mmol) at −4° C.; and the mixture was stirred at 0° C. for 15 minutes. 2-(1-(tert-butyldimethylsilyloxy)vinyl)-5-(trifluoromethyl)pyridine (1.40 g, 4.61 mmol) was added and the mixture was stirred at 0° C. for 2 hrs. The reaction mixture was quenched with aqueous saturated ammonium chloride (50 mL), and the aqueous layer was extracted with DCM (2×50 mL). The combined organic layers were washed by brine, dried over Na₂SO₄ and concentrated under reduced pressure. The residue was purified by flash column chromatography on silica (0-15% MTBE/petroleum ether) to give 2-(1-(tert-butyldimethylsilyloxy)cyclopropyl)-5-(trifluoromethyl)pyridine (450 mg, 30% yield) as yellow oil. LCMS (System 2, Method C) m/z 318.2 (M+H)⁺ (ES⁺).

### Step 5

A solution of 2-(1-(tert-butyldimethylsilyloxy)cyclopropyl)-5-(trifluoromethyl)pyridine (450 mg, 1.42 mmol) in HCl/dioxane (6 mL, 4.0 M solution) was stirred at room temperature for 1.5 hrs. The reaction mixture was concentrated under reduced pressure. The residue was purified by flash column chromatography on silica (0-15% MTBE/petroleum ether) to give 1-(5-(trifluoromethyl)pyridin-2-yl)cyclopropanol (250 mg, 86%) as yellow oil. LCMS (System 2, Method C) m/z 204.2 (M+H)⁺ (ES⁺).

### Intermediate 37—1-(5-(trifluoromethyl)pyrimidin-2-yl)cyclobutan-1-ol

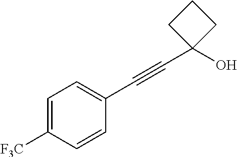 [SMILES: OC1(C#Cc2ccc(C(F)(F)F)cc2)CCC1]

Prepared by an analogous method to Intermediate 6 starting from 1-ethynyl-4-(trifluoromethyl)benzene (8.00 g, 47.0 mmol) and cyclobutanone (3.46 g, 49.37 mmol). Yield 6.50 g, 57%. Yellow oil. LCMS (System 2, Method C) m/z 223.2 (M-OH)⁺ (ES⁺).

### Intermediate 38—1-(6-(trifluoromethyl)pyridazin-3-yl)cyclobutan-1-ol

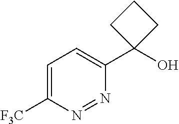 [SMILES: OC1(c2ccc(C(F)(F)F)nn2)CCC1]

Prepared by an analogous method to Intermediate 6 starting from 3-bromo-6-(trifluoromethyl)pyridazine (1.70 g, 7.49 mmol) and cyclobutanone (525 mg, 7.49 mmol). Yield 260 mg, 16%. Yellow oil. LCMS (System 2, Method C) m/z 219.2 (M+H)⁺ (ES⁺).

### Intermediate 39—1-(5-(trifluoromethyl)pyrazin-2-yl)cyclobutan-1-ol

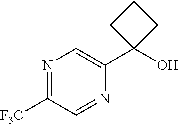 [SMILES: OC1(c2cnc(C(F)(F)F)cn2)CCC1]

Prepared by an analogous method to Intermediate 6 starting from 2-bromo-5-(trifluoromethyl)pyrazine (900 mg, 3.97 mmol) and cyclobutanone (278 mg, 3.97 mmol). Yield 0.45 g, 52%. Yellow oil. LCMS (System 2, Method C) m/z m/z 219.2 (M+H)⁺ (ES⁺).

### Example 1—2-methylene-4-oxo-4-(1-(4-(trifluoromethyl)phenyl)cyclobutoxy)butanoic acid

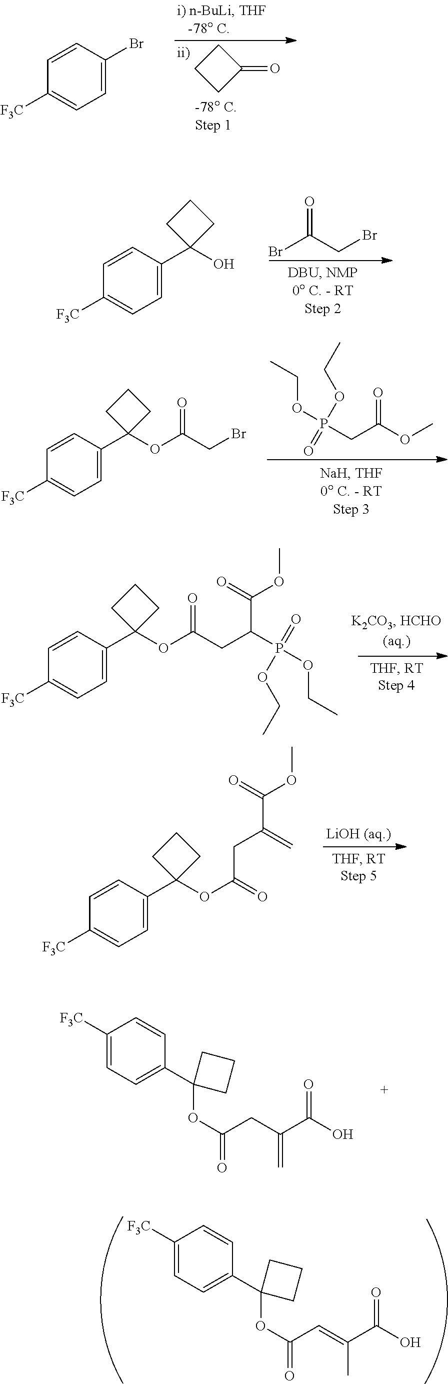

### Step 1

To a solution of 1-bromo-4-(trifluoromethyl)benzene (22.3 g, 99.5 mmol) in THF (180 mL) at −78° C. was added n-BuLi solution in hexane (2.5 M, 43.7 mL, 109.2 mmol) and the mixture was stirred at −78° C. for 1 h. Cyclobutanone (7.6 g, 109.2 mmol) was added, and the mixture was stirred at −78° C. for 5 h, then quenched with saturated aqueous NH₄Cl solution (200 mL). The phases were separated and the aqueous layer was extracted with MTBE (2×80 mL). The combined organic layers were washed with brine, dried over Na₂SO₄, filtered and concentrated under reduced pressure at 30° C., and the residue was purified by flash column chromatography (120 g silica, 0-14% MTBE/petroleum ether) to give 1-(4-(trifluoromethyl)phenyl)cyclobutan-1-ol (16.5 g, 76.3 mmol, 77%) as a yellow oil. ¹H NMR (400 MHz, CDCl₃) δ: 7.61 (s, 4H), 2.59-2.48 (m, 2H), 2.43-2.32 (m, 2H), 2.12-1.98 (m, 1H), 1.81-1.66 (m, 1H). One exchangable proton not observed.

### Step 2

To a solution of 1-(4-(trifluoromethyl)phenyl)cyclobutan-1-ol (16.5 g, 76.3 mmol) and DBU (23.2 g, 153.4 mmol) in 1-methyl-2-pyrrolidinone (300 mL) at 0° C. was slowly added 2-bromoacetyl bromide (30.9 g, 153.4 mmol) dropwise, and the mixture was stirred at room temperature for 16 h. The reaction mixture was diluted with water (160 mL) and MTBE (200 mL), the phases were separated, and the aqueous layer was extracted with MTBE (2×150 mL). The combined organic layers were washed with brine, dried over Na₂SO₄, filtered and concentrated under reduced pressure at 40° C. The residue was purified by flash column chromatography (40 g silica, 0-10% MTBE/petroleum ether) to give 1-(4-(trifluoromethyl)phenyl)cyclobutyl 2-bromoacetate (20 g, 59.3 mmol, 77%) as a yellow oil. ¹H NMR (400 MHz, CDCl₃) δ: 7.67-7.57 (m, 4H), 3.76 (s, 2H), 2.75-2.60 (m, 4H), 2.11-1.98 (m, 1H), 1.85-1.70 (m, 1H).

### Step 3

To a solution of methyl 2-(diethoxyphosphoryl)acetate (8.1 g, 38.3 mmol) in THF (150 mL) at 0° C. was added NaH suspension in mineral oil (60 wt. %, 1.5 g, 38.3 mmol) and the reaction mixture was stirred at 0° C. for 0.5 h. 1-(4-(Trifluoromethyl)phenyl)cyclobutyl 2-bromoacetate (10 g, 29.8 mmol) was then added and the reaction mixture was stirred at room temperature overnight. The reaction mixture was quenched with dilute aqueous HCl (0.5 M) and adjusted to pH=5. The phases were separated, and the aqueous layer was extracted with EtOAc (2×100 mL). The combined organic layers were washed with brine, dried over Na₂SO₄, filtered and concentrated under reduced pressure at 40° C. to give 1-methyl 4-(1-(4-(trifluoromethyl)phenyl)cyclobutyl) 2-(diethoxyphosphoryl)succinate (14.4 g, 30.9 mmol, >100%) as a colorless oil, which was used directly in the next step. LCMS (System 2, Method C) m/z 489.0 (M+Na)⁺ (ES⁺).

### Step 4

To a mixture of 1-methyl 4-(1-(4-(trifluoromethyl)phenyl)cyclobutyl) 2-(diethoxyphosphoryl)succinate (14.4 g, 30.9 mmmol) and potassium carbonate (8.5 g, 61.4 mmol) in THF (200 mL) at room temperature was added formaldehyde solution in water (37 wt. %, 16.3 mL, 153.6 mmol) and the reaction mixture was stirred at room temperature for 4 h. The reaction mixture was diluted with H₂O (150 mL) and extracted with MTBE (2×200 mL). The combined organic layers were washed with H₂O (2×100 mL) and brine, dried over Na₂SO₄, filtered and concentrated under reduced pressure at 30° C. The residue was purified by flash column chromatography (120 g silica, 0-10% MTBE/petroleum ether) to give 1-methyl 4-(1-(4-(trifluoromethyl)phenyl)cyclobutyl) 2-methylenesuccinate (6 g, 17.5 mmol, 56%) as a colorless oil. LCMS (System 2, Method C) m/z 365.0 (M+Na)⁺ (ES⁺).

### Step 5

To a solution of 1-methyl 4-(1-(4-(trifluoromethyl)phenyl)cyclobutyl) 2-methylenesuccinate (6 g, 17.6 mmol) in THF (30 mL) was added LiOH solution in water (2 M, 13.5 mL, 26.3 mmol), and the reaction mixture was stirred at room temperature for 6 h and then kept in a fridge at 0° C. overnight. The reaction mixture was acidified with dilute aqueous HCl (0.5 M) until pH=3 and extracted with EtOAc (2×10 mL). The combined EtOAc layers were washed with brine, dried over Na₂SO₄, filtered and concentrated under reduced pressure at 30° C. to give a residue comprising of a 2:1 mixture of 2-methylene-4-oxo-4-(1-(4-(trifluoromethyl)phenyl)cyclobutoxy)butanoic acid and 2-methyl-4-oxo-4-(1-(4-(trifluoromethyl)phenyl)cyclobutoxy)but-2-enoic acid. The mixture was purified by reversed phase column chromatography (330 g C18 silica; flow rate: 60 mL/min; 60-85% MeCN/(10 mM formic acid/water); collection wavelength: 214 nm). The collected fractions were concentrated under reduced pressure at 30° C. to remove MeCN, and the residue was lyophilized to give impure 2-methylene-4-oxo-4-(1-(4-(trifluoromethyl)phenyl)cyclobutoxy) butanoic acid (1.8 g, 5.48 mmol, 31%) as a white solid. A small quantity (100 mg) was further purified by preparative HPLC (Column: Waters X-Bridge C18 OBD 10 μm 19×250 mm; Flow Rate: 20 mL/min; solvent system: MeCN/(0.2% formic acid/water); gradient: 55-95% MeCN; collection wavelength: 214 nm). The collected fractions were concentrated under reduced pressure at 30° C. to remove MeCN, and the residue was lyophilized to give 2-methylene-4-oxo-4-(1-(4-(trifluoromethyl)phenyl)cyclobutoxy)butanoic acid (47 mg) as a white solid. LCMS (System 2, Method B) m/z 351.0 (M+Na)⁺ (ES⁺). ¹H NMR (400 MHz, DMSO-d6) δ: 12.65 (br, 1H), 7.71 (d, J=8.5 Hz, 2H), 7.65 (d, J=8.2 Hz, 2H), 6.13 (d, J=1.6 Hz, 1H), 5.74 (d, J=1.5 Hz, 1H), 3.33 (d, J=3.9 Hz, 2H), 2.61-2.50 (m, 4H), 2.02-1.89 (m, 1H), 1.84-1.69 (m, 1H).

As shown in the synthetic scheme for Example 1 above, Example 21 is obtained in Step 5 as a by-product. Full experimental details for this compound are provided below.

### Example 2—2-methylene-4-oxo-4-((2-(4-(trifluoromethyl)phenyl)propan-2-yl)oxy)butanoic acid

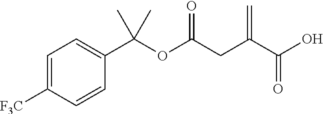 [SMILES: C=C(CC(=O)OC(C)(C)c1ccc(C(F)(F)F)cc1)C(=O)O]

Prepared by an analogous method to Example 1 starting from 2-(4-(trifluoromethyl)phenyl)propan-2-ol (8.0 g, 39.2 mmol) in Step 2, except that in Step 5 IPA was used as the solvent in place of THF and the reaction mixture was stirred at 10-15° C. for 2 h and then worked up. The crude product was purified by preparative HPLC (Column: Waters Sunfire Prep C18 OBD 10 μm 19×250 mm; Flow Rate: 20 mL/min; solvent system: MeCN/(0.2% formic acid/water); gradient: 50-95% MeCN; collection wavelength: 214 nm). The collected fractions were concentrated under reduced pressure at 25° C. to remove MeCN, and the residue was lyophilized to give 2-methylene-4-oxo-4-((2-(4-(trifluoromethyl)phenyl)propan-2-yl)oxy)butanoic acid (218 mg, 0.69 mmol) as a white solid. LCMS (System 2, Method B) m/z 339.0 (M+Na)⁺ (ES⁺). ¹H NMR (400 MHz, DMSO-d6) δ: 12.65 (br, 1H), 7.67 (d, J=8.3 Hz, 2H), 7.56 (d, J=8.2 Hz, 2H), 6.13 (d, J=1.6 Hz, 1H), 5.75 (d, J=1.5 Hz, 1H), 3.33 (t, J=3.2 Hz, 2H), 1.69 (s, 6H).

### Example 3—(R)-2-methylene-4-oxo-4-(1-(4-(trifluoromethyl)phenyl)ethoxy)butanoic acid

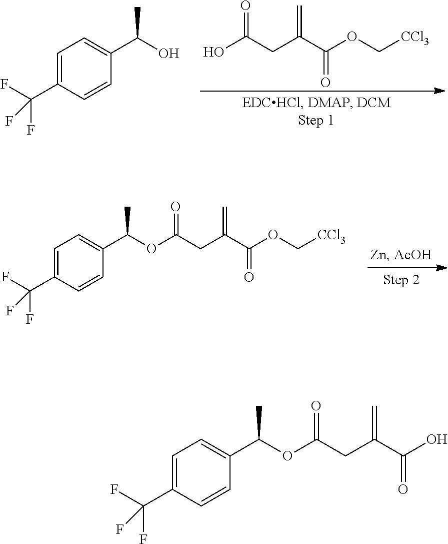

### Step 1

To a solution of (R)-1-(4-(trifluoromethyl)phenyl)ethan-1-ol (200 mg, 1.05 mmol), 3-((2,2,2-trichloroethoxy)carbonyl)but-3-enoic acid (Intermediate 1, 273 mg, 1.05 mmol) and DMAP (102 mg, 0.84 mmol) in DCM (4 mL) at 0° C. was added EDC·HCl (303 mg, 1.58 mmol), and the resulting pale-yellow mixture was stirred at room temperature for 20 min. The mixture was quenched with dilute aqueous HCl (0.5 M), separated and the aqueous phase was extracted with DCM (3×5 mL). The combined organic layers were washed with brine, dried over Na₂SO₄ and filtered. The filtrate was concentrated under reduced pressure at 25° C., and the residue was purified by flash column chromatography (20 g silica, 0-20% MTBE/petroleum ether) to give (R)-1-(2,2,2-trichloroethyl) 4-(1-(4-(trifluoromethyl)phenyl)ethyl) 2-methylenesuccinate (200 mg, 0.46 mmol, 44%) as a pale-yellow oil. LCMS: (System 2, Method C) m/z 454.8/456.8 (M+Na)⁺ (ES⁺).

### Step 2

A mixture of (R)-1-(2,2,2-trichloroethyl) 4-(1-(4-(trifluoromethyl)phenyl)ethyl) 2-methylenesuccinate (200 mg, 0.46 mmol) and zinc powder (150 mg, 2.32 mmol) in AcOH (2 mL) was stirred at room temperature for 2 days. The reaction mixture was filtered, and the filtrate was quenched with H₂O (3 mL), the phases were separated, and the aqueous layer was extracted with EtOAc (2×5 mL). The combined organic layers were washed with brine, dried over Na₂SO₄ and filtered. The filtrate was concentrated under reduced pressure at 25° C. and the residue was purified by preparative HPLC (Column: Waters Sunfire Prep C18 OBD 10 μm 19×250 mm; Flow Rate: 20 mL/min; solvent system: MeCN/(0.2% formic acid/water); gradient: 45-95% MeCN; collection wavelength: 214 nm). The collected fractions were concentrated under reduced pressure at 30° C. to remove MeCN, and the residue was lyophilized to give (R)-2-methylene-4-oxo-4-(1-(4-(trifluoromethyl)phenyl)ethoxy)butanoic acid (99 mg, 0.33 mmol, 71%) as a colorless oil. LCMS (System 2, Method B) m/z 324.9 (M+Na)⁺ (ES⁺). ¹H NMR (400 MHz, DMSO-d6) δ: 12.64 (br, 1H), 7.72 (d, J=8.1 Hz, 2H), 7.58 (d, J=8.1 Hz, 2H), 6.16 (d, J=1.6 Hz, 1H), 5.87 (q, J=6.6 Hz, 1H), 5.78 (d, J=1.5 Hz, 1H), 3.38 (s, 2H), 1.46 (d, J=6.6 Hz, 3H).

### Example 4—(S)-2-methylene-4-oxo-4-(1-(4-(trifluoromethyl)phenyl)ethoxy)butanoic acid

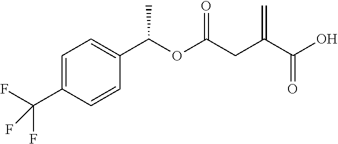 [SMILES: C=C(CC(=O)O[C@@H](C)c1ccc(C(F)(F)F)cc1)C(=O)O]

Prepared by an analogous method to Example 3 starting from (S)-1-(4-(trifluoromethyl)phenyl)ethan-1-ol (200 mg, 1.05 mmol). Yield: 79 mg, 0.26 mmol. Colorless oil. LCMS (System 2, Method B) m/z 324.9 (M+Na)⁺ (ES⁺). ¹H NMR (400 MHz, DMSO-d6) δ: 12.67 (br, 1H), 7.72 (d, J=8.1 Hz, 2H), 7.58 (d, J=8.1 Hz, 2H), 6.16 (d, J=1.6 Hz, 1H), 5.87 (q, J=6.6 Hz, 1H), 5.78 (d, J=1.5 Hz, 1H), 3.38 (s, 2H), 1.46 (d, J=6.6 Hz, 3H).

### Example 5—4-(1-(4-bromophenyl)cyclobutoxy)-2-methylene-4-oxobutanoic acid

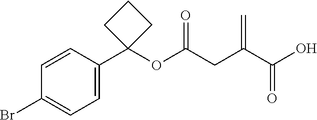 [SMILES: C=C(CC(=O)OC1(c2ccc(Br)cc2)CCC1)C(=O)O]

Prepared by an analogous method to Example 2 starting from 1-(4-bromophenyl)cyclobutan-1-ol (2.0 g, 8.8 mmol). Yield: 128 mg, 0.38 mmol. White solid. LCMS (System 2, Method B) m/z 360.8/362.8 (M+Na)⁺ (ES⁺). ¹H NMR (400 MHz, DMSO-d6) δ: 12.66 (br, 1H), 7.56-7.50 (m, 2H), 7.41-7.35 (m, 2H), 6.12 (d, J=1.6 Hz, 1H), 5.72 (s, 1H), 3.28 (s, 2H), 2.57-2.42 (m, 4H), 1.99-1.85 (m, 1H), 1.78-1.62 (m, 1H).

### Example 6—4-(1-(2-chloro-4-(trifluoromethyl)phenyl)cyclobutoxy)-2-methylene-4-oxobutanoic acid

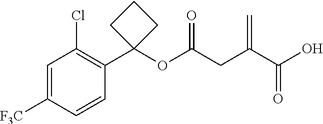 [SMILES: C=C(CC(=O)OC1(c2ccc(C(F)(F)F)cc2Cl)CCC1)C(=O)O]

Prepared by an analogous method to Example 1 starting from 1-bromo-2-chloro-4-(trifluoromethyl)benzene (5.0 g, 19 mmol) except that in Step 5 IPA was used as the solvent in place of THF and the reaction mixture was stirred at 10-15° C. for 2 h and then worked up. Yield: 117 mg, 0.32 mmol. White solid. LCMS (System 2, Method B) m/z 384.9/386.8 (M+Na)⁺ (ES⁺). ¹H NMR (400 MHz, DMSO-d6) δ: 12.60 (br, 1H), 7.90 (d, J=8.2 Hz, 1H), 7.81 (s, 1H), 7.71 (d, J=8.3 Hz, 1H), 6.10 (d, J=1.6 Hz, 1H), 5.70 (s, 1H), 3.23 (s, 2H), 2.84-2.72 (m, 2H), 2.66-2.54 (m, 2H), 2.04-1.91 (m, 1H), 1.71-1.57 (m, 1H).

### Example 7—2-methylene-4-oxo-4-((1-(4-(trifluoromethyl)phenyl)cyclopentyl)oxy)butanoic acid

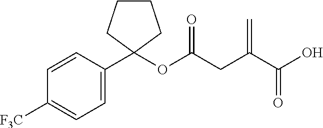 [SMILES: C=C(CC(=O)OC1(c2ccc(C(F)(F)F)cc2)CCCC1)C(=O)O]

Prepared by an analogous method to Example 6 starting from 1-bromo-4-(trifluoromethyl)benzene (4.0 g, 18.0 mmol) and cyclopentanone (1.51 g, 18.0 mmol). Yield: 125 mg, 0.37 mmol. White solid. LCMS (System 2, Method B) m/z 364.9 (M+Na)⁺ (ES⁺). ¹H NMR (400 MHz, DMSO-d6) δ: 12.68 (br, 1H), 7.65 (d, J=8.3 Hz, 2H), 7.55 (d, J=8.2 Hz, 2H), 6.12 (d, J=1.6 Hz, 1H), 5.74 (s, 1H), 3.31 (s, 2H), 2.32-2.21 (m, 2H), 2.11-1.98 (m, 2H), 1.86-1.69 (m, 4H).

### Example 8—4-(1-(4-chlorophenyl)cyclobutoxy)-2-methylene-4-oxobutanoic acid

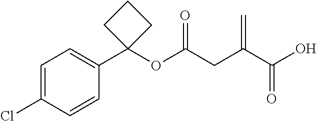 [SMILES: C=C(CC(=O)OC1(c2ccc(Cl)cc2)CCC1)C(=O)O]

Prepared by an analogous method to Example 2 starting from 1-(4-chlorophenyl)cyclobutan-1-ol (6.3 g, 35.5 mmol), except that Step 5 was stirred at 0° C. for 3 h. Yield: 159 mg, 0.54 mmol. White solid. LCMS (System 2, Method B) m/z 316.9/319.0 (M+Na)⁺ (ES⁺). ¹H NMR (400 MHz, DMSO-d6) δ: 12.62 (br, 1H), 7.45 (d, J=8.7 Hz, 2H), 7.39 (d, J=8.6 Hz, 2H), 6.12 (s, 1H), 5.72 (s, 1H), 3.28 (s, 2H), 2.56-2.43 (m, 4H), 1.98-1.86 (m, 1H), 1.78-1.62 (m, 1H).

### Example 9—2-methylene-4-oxo-4-(1-(4-((trifluoromethyl)thio)phenyl)cyclobutoxy)butanoic acid

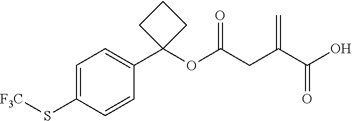 [SMILES: C=C(CC(=O)OC1(c2ccc(SC(F)(F)F)cc2)CCC1)C(=O)O]

Prepared by an analogous method to Example 2 starting from 1-(4-((trifluoromethyl)thio)phenyl)cyclobutan-1-ol (Intermediate 2, 2.5 g, 10 mmol). Yield: 84 mg, 0.23 mmol. Colorless oil. LCMS (System 2, Method B) m/z 382.9 (M+Na)⁺ (ES⁺). ¹H NMR (400 MHz, DMSO-d6) δ: 12.64 (br, 1H), 7.69 (d, J=8.2 Hz, 2H), 7.61-7.56 (m, 2H), 6.13 (d, J=1.6 Hz, 1H), 5.74 (s, 1H), 3.33 (s, 2H), 2.58-2.49 (m, 4H), 2.02-1.89 (m, 1H), 1.84-1.70 (m, 1H).

### Example 10—2-methylene-4-oxo-4-(1-(4-(trifluoromethyl)phenyl)propoxy)butanoic acid

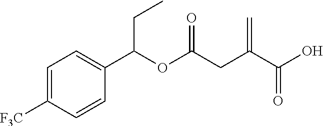 [SMILES: C=C(CC(=O)OC(CC)c1ccc(C(F)(F)F)cc1)C(=O)O]

Prepared by an analogous method to Example 2 starting from 1-(4-(trifluoromethyl)phenyl)propan-1-ol (0.5 g, 2.4 mmol). Yield: 59 mg, 0.19 mmol. White solid. LCMS (System 2, Method B) m/z 339.0 (M+Na)⁺ (ES⁺). ¹H NMR (400 MHz, DMSO-d6) δ: 12.66 (br, 1H), 7.71 (d, J=8.1 Hz, 2H), 7.54 (d, J=8.0 Hz, 2H), 6.16 (d, J=1.6 Hz, 1H), 5.79 (s, 1H), 5.69 (t, J=6.5 Hz, 1H), 3.46-3.34 (m, 2H), 1.85-1.72 (m, 2H) 0.83 (d, J=7.3 Hz, 3H).

### Example 11—2-methylene-4-oxo-4-(1-(4-(trifluoromethyl)phenyl)cyclopropoxy)butanoic acid

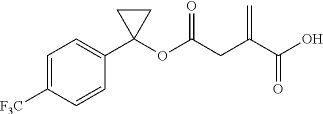 [SMILES: C=C(CC(=O)OC1(c2ccc(C(F)(F)F)cc2)CC1)C(=O)O]

Prepared by an analogous method to Example 2 starting from 1-(4-(trifluoromethyl)phenyl)cyclopropan-1-ol (0.5 g, 2.5 mmol). Yield: 39 mg, 0.12 mmol. White solid. LCMS (System 2, Method B) m/z 315.0 (M+H)⁺ (ES⁺). ¹H NMR (400 MHz, DMSO-d6) δ: 12.78 (br, 1H), 7.63 (d, J=8.2 Hz, 2H), 7.38 (d, J=8.2 Hz, 2H), 6.17 (d, J=1.6 Hz, 1H), 5.80 (s, 1H), 3.41 (s, 2H), 1.40-1.29 (m, 4H).

### Example 12—4-(1-(3-chloro-4-(trifluoromethyl)phenyl)cyclobutoxy)-2-methylene-4-oxobutanoic acid

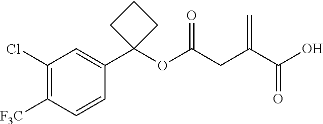 [SMILES: C=C(CC(=O)OC1(c2ccc(C(F)(F)F)c(Cl)c2)CCC1)C(=O)O]

Prepared by an analogous method to Example 2 starting from 1-(3-chloro-4-(trifluoromethyl)phenyl)cyclobutan-1-ol (Intermediate 3, 2.6 g, 10.4 mmol). Yield: 91 mg, 0.25 mmol. Yellow solid. LCMS (System 2, Method B) m/z 384.9 (M+Na)⁺ (ES⁺). ¹H NMR (400 MHz, DMSO-d6) δ: 12.68 (br, 1H), 7.83 (d, J=8.2 Hz, 1H), 7.70 (d, J=1.8 Hz, 1H), 7.59 (dd, J=8.1, 1.7 Hz, 1H), 6.14 (d, J=1.6 Hz, 1H), 5.76 (d, J=1.5 Hz, 1H), 3.34 (s, 2H), 2.63-2.53 (m, 2H), 2.53-2.45 (m, 2H), 2.03-1.89 (m, 1H), 1.86-1.71 (m, 1H).

### Example 13—2-methylene-4-oxo-4-((4-(trifluoromethyl)benzyl)oxy)butanoic acid

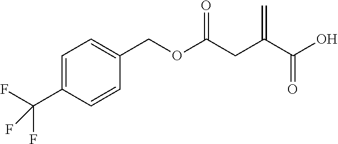 [SMILES: C=C(CC(=O)OCc1ccc(C(F)(F)F)cc1)C(=O)O]

Prepared by an analogous method to Example 3 starting from (4-(trifluoromethyl)phenyl)methanol (300 mg, 1.7 mmol), except that in Step 1 THF was used as the solvent in place of DCM. Yield: 162 mg, 0.56 mmol. White solid. LCMS (System 2, Method B) m/z 289.0 (M+H)⁺ (ES⁺). ¹H NMR (400 MHz, DMSO-d6) δ: 12.69 (br, 1H), 7.73 (d, J=8.0 Hz, 2H), 7.57 (d, J=8.0 Hz, 2H), 6.18 (d, J=1.6 Hz, 1H), 5.81 (s, 1H), 5.21 (s, 2H), 3.42 (s, 2H).

### Example 14—4-(1-(3-chloro-4-methylphenyl)cyclobutoxy)-2-methylene-4-oxobutanoic acid

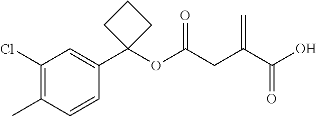 [SMILES: C=C(CC(=O)OC1(c2ccc(C)c(Cl)c2)CCC1)C(=O)O]

Prepared by an analogous method to Example 6 starting from 4-bromo-2-chloro-1-methylbenzene (4.0 g, 19.6 mmol). Yield: 159 mg, 0.51 mmol. White solid. LCMS (System 2, Method B) m/z 331.0 (M+Na)⁺ (ES⁺). ¹H NMR (400 MHz, DMSO-d6) δ: 12.65 (br, 1H), 7.41 (d, J=1.8 Hz, 1H), 7.35-7.26 (m, 2H), 6.12 (d, J=1.6 Hz, 1H), 5.73 (d, J=1.5 Hz, 1H), 3.28 (s, 2H), 2.58-2.40 (m, 4H), 2.31 (s, 3H), 1.98-1.84 (m, 1H), 1.77-1.61 (m, 1H).

### Example 15—2-methylene-4-oxo-4-(1-(4-(trifluoromethoxy)phenyl)cyclobutoxy)butanoic acid

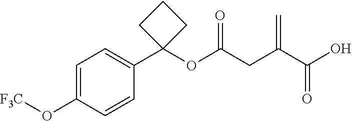 [SMILES: C=C(CC(=O)OC1(c2ccc(OC(F)(F)F)cc2)CCC1)C(=O)O]

Prepared by an analogous method to Example 6 starting from 1-bromo-4-(trifluoromethoxy)benzene (7.5 g, 31.3 mmol) except that Step 5 was stirred at room temperature for 4 h. Yield: 195 mg, 0.57 mmol. White solid. LCMS (System 2, Method B) m/z 366.9 (M+Na)⁺ (ES⁺). ¹H NMR (400 MHz, DMSO-d6) δ: 12.63 (br, 1H), 7.59-7.51 (m, 2H), 7.33 (d, J=8.3 Hz, 2H), 6.12 (d, J=1.6 Hz, 1H), 5.73 (d, J=1.5 Hz, 1H), 3.30 (s, 2H), 2.61-2.42 (m, 4H), 2.01-1.86 (m, 1H), 1.80-1.65 (m, 1H).

### Example 16—4-(1-(4′-fluoro-[1,1′-biphenyl]-4-yl)cyclobutoxy)-2-methylene-4-oxobutanoic acid

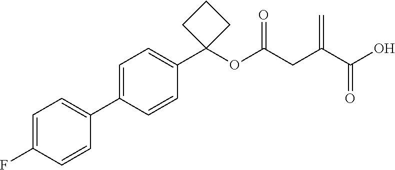 [SMILES: C=C(CC(=O)OC1(c2ccc(-c3ccc(F)cc3)cc2)CCC1)C(=O)O]

Prepared by an analogous method to Example 6 starting from 4-bromo-4′-fluoro-1,1′-biphenyl (5.0 g, 20.0 mmol) except that Step 5 was stirred at room temperature for 6 h and then at 0° C. overnight. Yield: 137 mg, 0.39 mmol. White solid. LCMS (System 2, Method B) m/z 376.9 (M+Na)⁺ (ES⁺). ¹H NMR (400 MHz, DMSO-d6) δ: 12.68 (br, 1H), 7.74-7.67 (m, 2H), 7.62 (d, J=8.2 Hz, 2H), 7.51 (d, J=8.4 Hz, 2H), 7.34-7.25 (m, 2H), 6.13 (d, J=1.6 Hz, 1H), 5.73 (s, 1H), 3.30 (s, 2H), 2.63-2.45 (m, 4H), 2.01-1.88 (m, 1H), 1.82-1.66 (m, 1H).

### Example 17—4-(1-(3-fluoro-4-(trifluoromethyl)phenyl)cyclobutoxy)-2-methylene-4-oxobutanoic acid

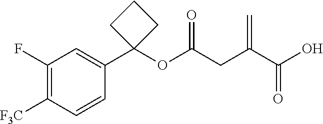 [SMILES: C=C(CC(=O)OC1(c2ccc(C(F)(F)F)c(F)c2)CCC1)C(=O)O]

Prepared by an analogous method to Example 2 starting from 1-(3-fluoro-4-(trifluoromethyl)phenyl)cyclobutan-1-ol (Intermediate 4, 3.65 g, 15.6 mmol). Yield: 122 mg, 0.35 mmol. White solid. LCMS (System 2, Method B) m/z 369.0 (M+Na)⁺ (ES⁺). ¹H NMR (400 MHz, DMSO-d6) δ: 12.69 (br, 1H), 7.76 (t, J=7.9 Hz, 1H), 7.52 (d, J=12.2 Hz, 1H), 7.47 (d, J=8.2 Hz, 1H), 6.14 (s, 1H), 5.77 (s, 1H), 3.35 (s, 2H), 2.62-2.44 (m, 4H), 2.03-1.89 (m, 1H), 1.88-1.72 (m, 1H).

### Example 18—2-methylene-4-oxo-4-(1-(4-(pentafluoro-λ⁶-sulfaneyl)phenyl)cyclobutoxy)butanoic acid

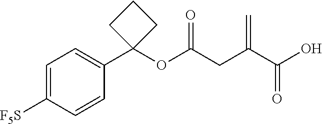

Prepared by an analogous method to Example 2 starting from 1-(4-(pentafluoro-λ⁶-sulfaneyl)phenyl)cyclobutan-1-ol (Intermediate 5, 1.9 g, 6.9 mmol). Yield: 81 mg, 0.21 mmol. White solid. LCMS (System 2, Method B) m/z 408.8 (M+Na)⁺ (ES⁺). ¹H NMR (400 MHz, DMSO-d6) δ: 12.71 (br, 1H), 7.90-7.83 (m, 2H), 7.65 (d, J=8.5 Hz, 2H), 6.13 (d, J=1.6 Hz, 1H), 5.75 (s, 1H), 3.34 (s, 2H), 2.62-2.44 (m, 4H), 2.03-1.89 (m, 1H), 1.85-1.69 (m, 1H).

### Example 19—4-(1-(4-(1,1-difluoroethyl)phenyl)cyclobutoxy)-2-methylene-4-oxobutanoic acid

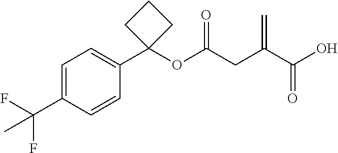 [SMILES: C=C(CC(=O)OC1(c2ccc(C(C)(F)F)cc2)CCC1)C(=O)O]

Prepared by an analogous method to Example 6 starting from 1-bromo-4-(1,1-difluoroethyl)benzene (3.0 g, 13.6 mmol). Yield: 169 mg, 0.52 mmol. White solid. LCMS (System 2, Method B) m/z 347.0 (M+Na)⁺ (ES⁺). ¹H NMR (400 MHz, DMSO-d6) δ: 12.63 (br, 1H), 7.53 (s, 4H), 6.13 (d, J=1.6 Hz, 1H), 5.73 (d, J=1.5 Hz, 1H), 3.31 (s, 2H), 2.61-2.43 (m, 4H), 2.05-1.87 (m, 4H), 1.83-1.66 (m, 1H).

### Example 20—4-(1-(4-(difluoromethyl)phenyl)cyclobutoxy)-2-methylene-4-oxobutanoic acid

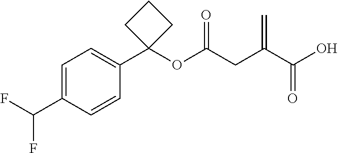 [SMILES: C=C(CC(=O)OC1(c2ccc(C(F)F)cc2)CCC1)C(=O)O]

Prepared by an analogous method to Example 6 starting from 1-bromo-4-(difluoromethyl)benzene (4.2 g, 20.5 mmol). Yield: 148 mg, 0.48 mmol. White solid. LCMS (System 2, Method B) m/z 333.0 (M+Na)⁺ (ES⁺). ¹H NMR (400 MHz, DMSO-d6) δ: 12.65 (br, 1H), 7.60-7.51 (m, 4H), 7.03 (t, J=55.9 Hz, 1H), 6.12 (d, J=1.6 Hz, 1H), 5.73 (s, 1H), 3.31 (s, 2H), 2.61-2.43 (m, 4H), 2.02-1.87 (m, 1H), 1.83-1.67 (m, 1H).

### Example 21—(E)-2-methyl-4-oxo-4-(1-(4-(trifluoromethyl)phenyl)cyclobutoxy)but-2-enoic acid

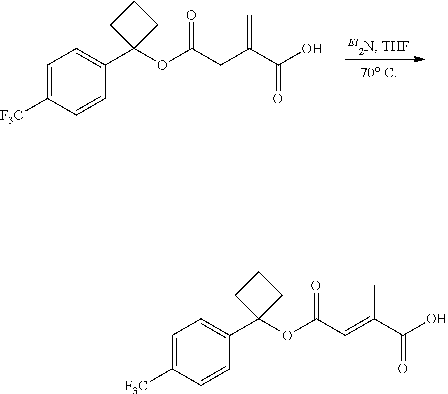 [SMILES: C=C(CC(=O)OC1(c2ccc(C(F)(F)F)cc2)CCC1)C(=O)O ; C/C(=C\C(=O)OC1(c2ccc(C(F)(F)F)cc2)CCC1)C(=O)O]

A mixture of 2-methylene-4-oxo-4-(1-(4-(trifluoromethyl)phenyl)cyclobutoxy)butanoic acid (Example 1, 1.00 g, 3.05 mmol) and triethylamine (926 mg, 9.15 mmol) in THF (10 mL) was stirred at 70° C. for 48 h. The reaction mixture was concentrated under reduced pressure at 35° C. and the residue was diluted with ethyl acetate (5 mL) and acidified to pH 3 with dilute aqueous HCl (0.5 M). The phases were separated, and the aqueous layer was extracted with ethyl acetate (2×5 mL). The combined organic layers were concentrated under reduced pressure at 30° C. to give crude (E)-2-methyl-4-oxo-4-(1-(4-(trifluoromethyl)phenyl)cyclobutoxy)but-2-enoic acid (800 mg, 80%). 300 mg of the crude material was purified by preparative HPLC (Column: Waters X-Bridge C18 OBD 10 μm 19×250 mm; Flow Rate: 20 mL/min; solvent system: MeCN/(0.2% formic acid/water); gradient: 58-95% MeCN; collection wavelength: 214 nm). The collected fractions were concentrated under reduced pressure at 30° C. to remove MeCN, and the residue was lyophilized to give (E)-2-methyl-4-oxo-4-(1-(4-(trifluoromethyl)phenyl)cyclobutoxy)but-2-enoic acid (139 mg, 0.42 mmol) as a white solid. LCMS (System 2, Method B) m/z 326.9 (M−H)⁻ (ES⁻). ¹H NMR (400 MHz, DMSO-ds) 5: 13.27 (br, 1H), 7.78-7.66 (m, 4H), 6.66 (d, J=1.6 Hz, 1H), 2.67-2.57 (m, 4H), 2.07 (d, J=1.6 Hz, 3H), 2.05-1.92 (m, 1H), 1.85-1.69 (m, 1H).

### Example 22—2-methylene-4-oxo-4-(1-(6-(trifluoromethyl)pyridin-3-yl)cyclobutoxy)butanoic acid

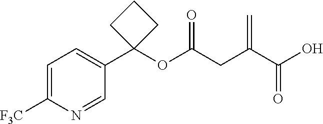 [SMILES: C=C(CC(=O)OC1(c2ccc(C(F)(F)F)nc2)CCC1)C(=O)O]

Prepared by an analogous method to Example 15 starting from 5-bromo-2-(trifluoromethyl)pyridine (3.0 g, 13.3 mmol). Yield: 70 mg, 0.21 mmol. White solid. LCMS (System 2, Method B) m/z 330.0 (M+H)⁺ (ES⁺). ¹H NMR (400 MHz, DMSO-d6) δ: 12.72 (br, 1H), 8.84 (d, J=2.3 Hz, 1H), 8.12 (dd, J=8.1, 2.4 Hz, 1H), 7.89 (d, J=8.2 Hz, 1H), 6.14 (d, J=1.6 Hz, 1H), 5.76 (s, 1H), 3.35 (s, 2H), 2.71-2.45 (m, 4H), 2.06-1.91 (m, 1H), 1.88-1.72 (m, 1H).

### Example 23—4-((3-(4-bromophenyl)oxetan-3-yl)oxy)-2-methylene-4-oxobutanoic acid

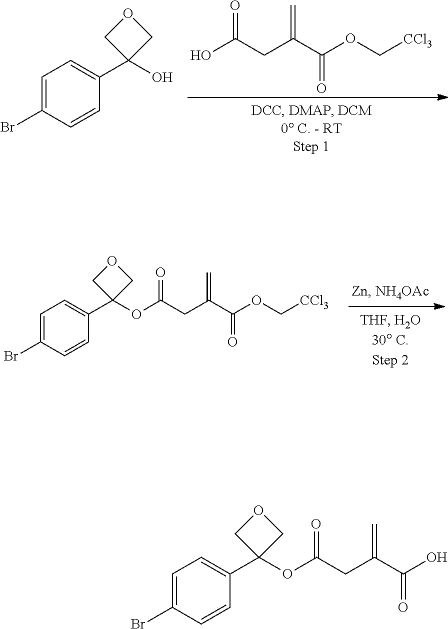

### Step 1

To a solution of 3-(4-bromophenyl)oxetan-3-ol (300 mg, 1.32 mmol), 3-((2,2,2-trichloroethoxy)carbonyl)but-3-enoic acid (Intermediate 1, 378 mg, 1.45 mmol) and DCC (408 mg, 1.98 mmol) in DCM (6 mL) was added DMAP (32 mg, 0.26 mmol) at 0° C., and the mixture was stirred at room temperature for 1 h. The reaction mixture was filtered and concentrated under reduced pressure at 35° C. The residue was purified by flash column chromatography (20 g silica, 0-22% MTBE/petroleum ether) to give 4-(3-(4-bromophenyl)oxetan-3-yl) 1-(2,2,2-trichloroethyl) 2-methylenesuccinate (300 mg, 0.64 mmol, 48%) as a pale-yellow oil. LCMS (System 2, Method B) m/z 492.6/494.6/496.6 (M+Na)⁺ (ES⁺).

### Step 2

To a solution of 4-(3-(4-bromophenyl)oxetan-3-yl) 1-(2,2,2-trichloroethyl) 2-methylenesuccinate (300 mg, 0.64 mmol) and ammonium acetate (493 mg, 6.40 mmol) in THF:H₂O (4:1, 3 mL) was added zinc powder (208 mg, 3.20 mmol), and the reaction mixture was stirred at 30° C. for 3 h. The reaction mixture was filtered and the filtrate was acidified with acetic acid to pH=5, and extracted with MTBE (2×5 mL). The combined organic layers were washed with brine, dried over Na₂SO₄, filtered and concentrated at under reduced pressure 30° C. The residue was purified by preparative HPLC (Column: Waters Sunfire Prep C18 OBD 10 μm 19×250 mm; Flow Rate: 20 mL/min; solvent system: MeCN/(0.05% formic acid/water); gradient: 40-95% MeCN; collection wavelength: 214 nm). The collected fractions were concentrated under reduced pressure at 35° C. to remove MeCN, and the residue was lyophilized to give 4-((3-(4-bromophenyl)oxetan-3-yl)oxy)-2-methylene-4-oxobutanoic acid (72 mg, 0.21 mmol, 33%) as a colorless oil. LCMS (System 2, Method B) m/z 362.8/364.8 (M+Na)⁺ (ES⁺). ¹H NMR (400 MHz, DMSO-d6) δ: 12.79 (br, 1H), 7.63-7.57 (m, 2H), 7.50-7.44 (m, 2H), 6.18 (d, J=1.6 Hz, 1H), 5.81 (s, 1H), 4.88 (d, J=7.7 Hz, 2H), 4.78 (d, J=7.9 Hz, 2H), 3.46 (s, 2H).

### Example 24—2-methylene-4-oxo-4-((3-(4-(trifluoromethyl)phenyl)oxetan-3-yl)oxy)butanoic acid

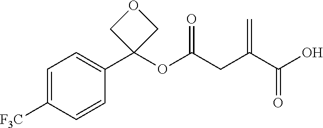 [SMILES: C=C(CC(=O)OC1(c2ccc(C(F)(F)F)cc2)COC1)C(=O)O]

Prepared by an analogous method to Example 23 starting from 3-(4-(trifluoromethyl)phenyl)oxetan-3-ol (Intermediate 6, 330 mg, 1.51 mmol). Yield: 132 mg, 0.40 mmol. White solid. LCMS (System 2, Method B) m/z 331.0 (M+H)⁺ (ES⁺). ¹H NMR (400 MHz, DMSO-d6) δ: 12.81 (s, 1H), 7.80-7.72 (m, 4H), 6.19 (s, 1H), 5.82 (s, 1H), 4.91 (d, J=7.7 Hz, 2H), 4.82 (d, J=7.7 Hz, 2H), 3.50 (s, 2H).

### Example 25—4-(1-(4-cyanophenyl)cyclobutoxy)-2-methylene-4-oxobutanoic acid

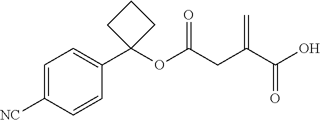 [SMILES: C=C(CC(=O)OC1(c2ccc(C#N)cc2)CCC1)C(=O)O]

Prepared by an analogous method to Example 23 starting from 4-(1-hydroxycyclobutyl)benzonitrile (Intermediate 7, 300 mg, 1.73 mmol). Yield: 77 mg, 0.27 mmol. White solid. LCMS (System 2, Method B) m/z 307.9 (M+Na)⁺ (ES⁺). 1H NMR (400 MHz, DMSO-d6) δ: 12.71 (br, 1H), 7.85-7.79 (m, 2H), 7.65-7.59 (m, 2H), 6.13 (d, J=1.6 Hz, 1H), 5.74 (s, 1H), 3.32 (s, 2H), 2.59-2.45 (m, 4H), 2.02-1.89 (m, 1H), 1.84-1.69 (m, 1H).

### Example 26—2-methylene-4-oxo-4-(1-(4′-(trifluoromethyl)-[1,1′-biphenyl]-4-yl)cyclobutoxy)butanoic acid

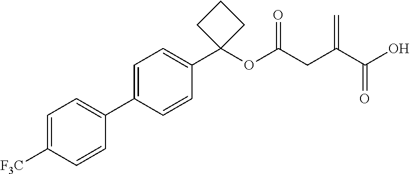 [SMILES: C=C(CC(=O)OC1(c2ccc(-c3ccc(C(F)(F)F)cc3)cc2)CCC1)C(=O)O]

Prepared by an analogous method to Example 23 starting from 1-(4′-(trifluoromethyl)-[1,1-biphenyl]-4-yl)cyclobutan-1-ol (Intermediate 8, 300 mg, 1.03 mmol). Yield: 35 mg, 0.09 mmol. White solid. LCMS (System 2, Method B) m/z 426.9 (M+Na)⁺ (ES⁺). ¹H NMR (400 MHz, DMSO-d6) δ: 12.67 (br, 1H), 7.90 (d, J=8.1 Hz, 2H), 7.82 (d, J=8.3 Hz, 2H), 7.72 (d, J=8.4 Hz, 2H), 7.56 (d, J=8.4 Hz, 2H), 6.13 (d, J=1.6 Hz, 1H), 5.74 (s, 1H), 3.32 (s, 2H), 2.64-2.49 (m, 4H), 2.02-1.89 (m, 1H), 1.84-1.68 (m, 1H).

### Example 27—2-methylene-4-oxo-4-(1-(5-(trifluoromethyl)pyridin-2-yl)cyclobutoxy)butanoic acid

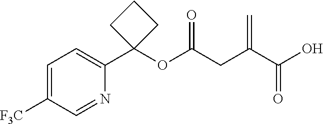 [SMILES: C=C(CC(=O)OC1(c2ccc(C(F)(F)F)cn2)CCC1)C(=O)O]

Prepared by an analogous method to Example 23 starting from 1-(5-(trifluoromethyl)pyridin-2-yl)cyclobutan-1-ol (Intermediate 9, 250 mg, 1.15 mmol). Yield: 102 mg, 0.31 mmol. White solid. LCMS (System 2, Method B) m/z 330.0 (M+H)⁺ (ES⁺). ¹H NMR (400 MHz, DMSO-d6) δ: 12.74 (br, 1H), 8.98 (s, 1H), 8.14 (dd, J=8.5, 2.4 Hz, 1H), 7.59 (d, J=8.4 Hz, 1H), 6.16 (d, J=1.6 Hz, 1H), 5.79 (d, J=1.4 Hz, 1H), 3.41 (s, 2H), 2.73-2.62 (m, 2H), 2.50-2.41 (m, 2H), 2.03-1.85 (m, 2H).

### Example 28—4-(1-(4-(difluoro(phenyl)methyl)phenyl)cyclobutoxy)-2-methylene-4-oxobutanoic acid

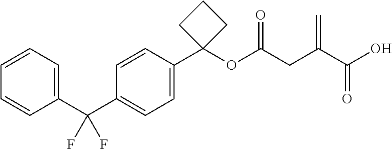 [SMILES: C=C(CC(=O)OC1(c2ccc(C(F)(F)c3ccccc3)cc2)CCC1)C(=O)O]

Prepared by an analogous method to Example 23 starting from 1-(4-(difluoro(phenyl)methyl)phenyl)cyclobutan-1-ol (Intermediate 10, 320 mg, 1.17 mmol). Yield: 77 mg, 0.20 mmol. White solid. LCMS (System 2, Method B) m/z 408.9 (M+Na)⁺ (ES⁺). ¹H NMR (400 MHz, DMSO-d6) δ: 12.68 (br, 1H), 7.58-7.45 (m, 9H), 6.12 (d, J=1.7 Hz, 1H), 5.73 (s, 1H), 3.30 (s, 2H), 2.59-2.43 (m, 4H), 2.00-1.86 (m, 1H), 1.82-1.66 (m, 1H).

### Example 29—4-(3,3-difluoro-1-(4-(trifluoromethyl)phenyl)cyclobutoxy)-2-methylene-4-oxobutanoic acid

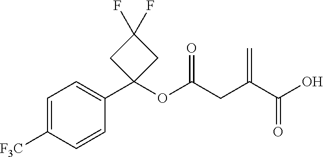 [SMILES: C=C(CC(=O)OC1(c2ccc(C(F)(F)F)cc2)CC(F)(F)C1)C(=O)O]

Prepared by an analogous method to Example 23 starting from 3,3-difluoro-1-(4-(trifluoromethyl)phenyl)cyclobutan-1-ol (Intermediate 11, 380 mg, 1.50 mmol). Yield: 51 mg, 0.14 mmol. White solid. LCMS (System 2, Method B) m/z 386.8 (M+Na)⁺ (ES⁺). 1H NMR (400 MHz, DMSO-d6) δ: 12.73 (br, 1H), 7.79-7.62 (m, 4H), 6.15 (d, J=1.5 Hz 1H), 5.77 (s, 1H), 3.49-3.31 (m, 2H), 3.37 (s, 2H), 3.28-3.12 (m, 2H).

### Example 30—3,3-dimethyl-2-methylene-4-oxo-4-(1-(4-(trifluoromethyl)phenyl)cyclobutoxy) butanoic acid

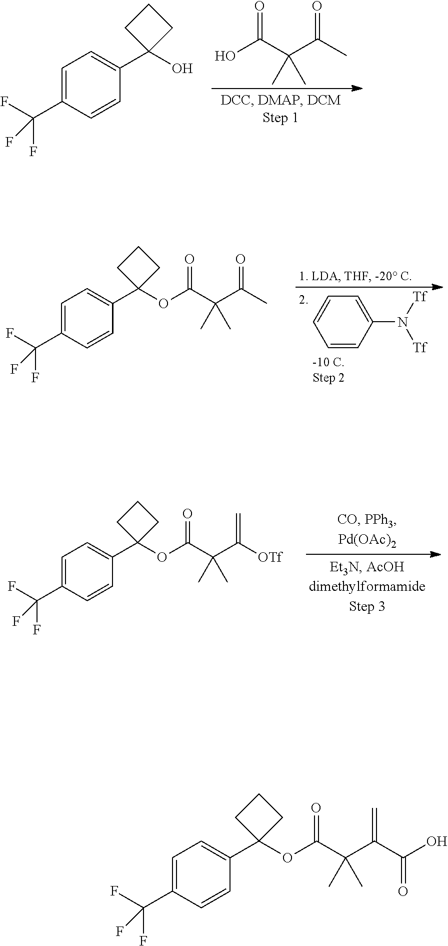

### Step 1

To a solution of 2,2-dimethyl-3-oxobutanoic acid (1.50 g, 11.53 mmol) and 1-(4-(trifluoromethyl)phenyl)cyclobutan-1-ol (from Example 1, 2.74 g, 12.68 mmol) in DCM (50 mL) at room temperature was added DCC (3.56 g, 17.29 mmol) and DMAP (281 mg, 2.30 mmol) and the mixture was stirred at room temperature for 16 h. The reaction mixture was filtered, and the filtrate was diluted with water (50 mL) and DCM (50 mL) and the phases were separated. The aqueous layer was extracted with DCM (2×50 mL), and the combined organic layers were washed with brine, dried over Na₂SO₄, filtered and concentrated under reduced pressure at 30° C. The residue was purified by flash column chromatography (40 g silica, 0-20% MTBE/petroleum ether) to give 1-(4-(trifluoromethyl)phenyl)cyclobutyl 2,2-dimethyl-3-oxobutanoate (600 mg, 1.83 mmol, 15%) as a yellow oil. LCMS: (System 2, Method C) m/z 351.0 (M+Na)⁺ (ES⁺).

### Step 2

LDA solution in THF/heptanes/ethylbenzene (2 M, 1.8 mL, 3.60 mmol) was added to THF (30 mL) at −20° C. then 1-(4-(trifluoromethyl)phenyl)cyclobutyl 2,2-dimethyl-3-oxobutanoate (600 mg, 1.83 mmol) was added and the reaction mixture was stirred at −20° C. for 10 minutes. 1,1,1-trifluoro-N-phenyl-N-((trifluoromethyl)sulfonyl)methanesulfonamide (1.37 g, 3.84 mmol) was then added and the reaction mixture was stirred at −10° C. for 2 h. The reaction mixture was quenched by the addition of saturated aqueous NaHCO₃ solution (30 mL) and extracted with MTBE (2×30 mL). The combined organic layers were dried over Na₂SO₄, filtered and concentrated under reduced pressure at 30° C. The residue was purified by flash column chromatography (25 g silica, 0-5% MTBE/petroleum ether) to give 1-(4-(trifluoromethyl)phenyl)cyclobutyl 2,2-dimethyl-3-(((trifluoromethyl)sulfonyl)oxy)but-3-enoate (390 mg, 0.85 mmol, 46%) as a yellow oil. LCMS: (System 2, Method C) m/z 482.8 (M+Na)⁺ (ES⁺).

### Step 3

A mixture of 1-(4-(trifluoromethyl)phenyl)cyclobutyl 2,2-dimethyl-3-(((trifluoromethyl)sulfonyl)oxy)but-3-enoate (390 mg, 0.85 mmol), triphenylphosphine (89 mg, 0.34 mmol) and palladium(II) acetate (38 mg, 0.17 mmol) in dimethylformamide (10 mL) was charged with CO gas. Separately, a solution of triethylamine (172 mg, 1.70 mmol) in dimethylformamide (20 mL) was cooled to 0° C., and to this solution was added AcOH (82 mg, 1.36 mmol), after which it was added to the first mixture and the whole charged with CO gas and stirred under a balloon containing CO gas at room temperature overnight. The reaction mixture was diluted with aqueous NaHCO₃ solution (20 mL) and extracted with EtOAc (2×10 mL). The combined organic layers were washed with saturated brine, dried over Na₂SO₄ and filtered. The filtrate was concentrated under reduced pressure at 25° C. and the residue was purified by preparative HPLC (Column: Waters Sunfire Prep C18 OBD 10 μm 19×250 mm; Flow Rate: 20 mL/min; solvent system: MeCN/(0.2% formic acid/water); gradient: 60-95% MeCN; collection wavelength: 214 nm). The collected fractions were concentrated under reduced pressure at 30° C. to remove MeCN, and the residue was lyophilized to give 3,3-dimethyl-2-methylene-4-oxo-4-(1-(4-(trifluoromethyl)phenyl)cyclobutoxy)butanoic acid (148 mg, 48%) as a white solid. LCMS (System 2, Method B) m/z 378.9 (M+Na)⁺ (ES⁺). ¹H NMR (400 MHz, DMSO-d6) δ: 12.81 (br, 1H), 7.71 (d, J=8.2 Hz, 2H), 7.60 (d, J=8.2 Hz, 2H), 6.22 (s, 1H), 5.80 (s, 1H), 2.56-2.40 (m, 4H), 1.96-1.82 (m, 1H), 1.80-1.65 (m, 1H), 1.26 (s, 6H).

### Example 31—2-methylene-4-oxo-4-(1-(4-(perfluoroethyl)phenyl)cyclobutoxy)butanoic acid

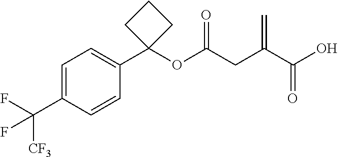 [SMILES: C=C(CC(=O)OC1(c2ccc(C(F)(F)C(F)(F)F)cc2)CCC1)C(=O)O]

Prepared by an analogous method to Example 23 starting from 1-(4-(perfluoroethyl)phenyl)cyclobutan-1-ol (Intermediate 12, 200 mg, 0.75 mmol). Yield: 41 mg, 0.11 mmol. White solid. LCMS (System 2, Method B) m/z 400.9 (M+Na)⁺ (ES⁺). ¹H NMR (400 MHz, DMSO-d6) δ: 12.70 (br, 1H), 7.68 (s, 4H), 6.13 (d, J=1.6 Hz, 1H), 5.75 (d, J=1.5 Hz, 1H), 3.33 (s, 2H), 2.63-2.47 (m, 4H), 2.04-1.89 (m, 1H), 1.87-1.70 (m, 1H).

### Example 32—2-methylene-4-oxo-4-(1-(5-(trifluoromethyl)thiophen-2-yl)cyclobutoxy)butanoic acid

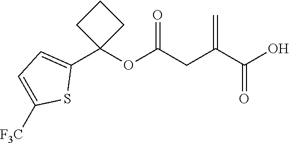 [SMILES: C=C(CC(=O)OC1(c2ccc(C(F)(F)F)s2)CCC1)C(=O)O]

Prepared by an analogous method to Example 23 starting from 1-(5-(trifluoromethyl)thiophen-2-yl)cyclobutan-1-ol (Intermediate 13, 200 mg, 0.90 mmol). Yield: 75 mg, 0.22 mmol. White solid. LCMS (System 2, Method B) m/z 356.8 (M+Na)⁺ (ES⁺). ¹H NMR (400 MHz, DMSO-d6) δ: 12.70 (br, 1H), 7.62-7.58 (m, 1H), 7.33-7.28 (m, 1H), 6.14 (d, J=1.6 Hz, 1H), 5.77 (d, J=1.4 Hz, 1H), 3.32 (s, 2H), 2.63-2.53 (m, 4H), 1.99-1.86 (m, 1H), 1.86-1.71 (m, 1H).

### Example 33—(S)-2-methylene-4-oxo-4-(2,2,2-trifluoro-1-(4-(trifluoromethyl)phenyl)ethoxy)butanoic acid

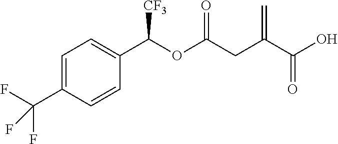 [SMILES: C=C(CC(=O)O[C@@H](c1ccc(C(F)(F)F)cc1)C(F)(F)F)C(=O)O]

Prepared by an analogous method to Example 23 starting from (S)-2,2,2-trifluoro-1-(4-(trifluoromethyl)phenyl)ethan-1-ol (Intermediate 14, 200 mg, 0.82 mmol). Yield: 104 mg, 0.29 mmol. White solid. LCMS (System 2, Method B) m/z 356.9 (M+H)⁺ (ES⁺). ¹H NMR (400 MHz, DMSO-d6) δ: 12.77 (br, 1H), 7.85 (d, J=8.2 Hz, 2H), 7.77 (d, J=8.4 Hz, 2H), 6.66 (q, J=6.9 Hz, 1H), 6.21 (d, J=1.6 Hz, 1H), 5.86 (d, J=1.4 Hz, 1H), 3.55 (s, 2H).

### Example 34—(R)-2-methylene-4-oxo-4-(2,2,2-trifluoro-1-(4-(trifluoromethyl)phenyl)ethoxy)butanoic acid

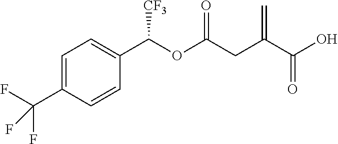 [SMILES: C=C(CC(=O)O[C@H](c1ccc(C(F)(F)F)cc1)C(F)(F)F)C(=O)O]

Prepared by an analogous method to Example 23 starting from (R)-2,2,2-trifluoro-1-(4-(trifluoromethyl)phenyl)ethan-1-ol (Intermediate 15, 200 mg, 0.82 mmol). Yield: 104 mg, 0.29 mmol. White solid. LCMS (System 2, Method B) m/z 357.0 (M+H)⁺ (ES⁺). ¹H NMR (400 MHz, DMSO-d6) δ: 12.77 (br, 1H), 7.85 (d, J=8.3 Hz, 2H), 7.77 (d, J=8.2 Hz, 2H), 6.65 (q, J=6.9 Hz, 1H), 6.21 (d, J=1.5 Hz, 1H), 5.86 (d, J=1.5 Hz, 1H), 3.55 (s, 2H).

### Example 35—2-methylene-4-oxo-4-((2-(4-(trifluoromethyl)phenyl)spiro[3.3]heptan-2-yl)oxy)butanoic acid

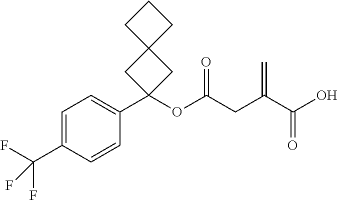 [SMILES: C=C(CC(=O)OC1(c2ccc(C(F)(F)F)cc2)CC2(CCC2)C1)C(=O)O]

Prepared by an analogous method to Example 23 starting from 2-(4-(trifluoromethyl)phenyl)spiro[3.3]heptan-2-ol (Intermediate 16, 300 mg, 1.17 mmol). Yield: 113 mg, 0.31 mmol. White solid. LCMS (System 2, Method B) m/z 390.9 (M+Na)⁺ (ES⁺). ¹H NMR (400 MHz, DMSO-d6) δ: 12.65 (br, 1H), 7.68 (d, J=8.2 Hz, 2H), 7.58 (d, J=8.2 Hz, 2H), 6.12 (d, J=1.6 Hz, 1H), 5.72 (s, 1H), 3.28 (s, 2H), 2.71 (d, J=13.5 Hz, 2H), 2.57-2.46 (m, 2H), 2.07 (t, J=7.5 Hz, 2H), 1.90 (t, J=7.4 Hz, 2H), 1.82-1.71 (m, 2H).

### Example 36—2-methylene-4-oxo-4-(1-(4-(3,3,3-trifluoroprop-1-yn-1-yl)phenyl)cyclobutoxy)butanoic acid

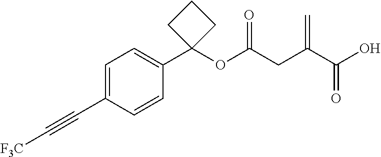 [SMILES: C=C(CC(=O)OC1(c2ccc(C#CC(F)(F)F)cc2)CCC1)C(=O)O]

Prepared by an analogous method to Example 23 starting from 1-(4-(3,3,3-trifluoroprop-1-yn-1-yl)phenyl)cyclobutan-1-ol (Intermediate 17, 140 mg, 0.58 mmol). Yield: 41 mg, 0.12 mmol. White solid. LCMS (System 2, Method B) m/z 375.0 (M+Na)⁺ (ES⁺). ¹H NMR (400 MHz, DMSO-d6) δ: 12.67 (br, 1H), 7.71 (d, J=8.4 Hz, 2H), 7.55 (d, J=8.4 Hz, 2H), 6.13 (d, J=1.6 Hz, 1H), 5.74 (d, J=1.5 Hz, 1H), 3.32 (s, 2H), 2.61-2.43 (m, 4H), 2.02-1.88 (m, 1H), 1.83-1.68 (m, 1H).

### Example 37—2-methylene-4-oxo-4-((1-(4-(trifluoromethyl)phenyl)prop-2-yn-1-yl)oxy)butanoic acid (Enantiomer 1)

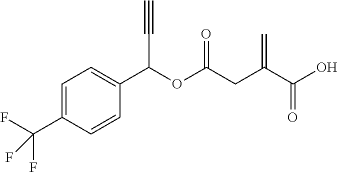 [SMILES: C#CC(OC(=O)CC(=C)C(=O)O)c1ccc(C(F)(F)F)cc1]

Prepared by an analogous method to Example 23 starting from 1-(4-(trifluoromethyl)phenyl)prop-2-yn-1-ol Enantiomer 1 (Intermediate 18, 230 mg, 1.15 mmol). Yield: 87 mg, 0.28 mmol. White solid. LCMS (System 2, Method B) m/z 334.9 (M+Na)⁺ (ES⁺). ¹H NMR (400 MHz, DMSO-d6) δ: 12.70 (br, 1H), 7.80 (d, J=8.2 Hz, 2H), 7.71 (d, J=8.1 Hz, 2H), 6.55 (d, J=2.3 Hz, 1H), 6.17 (d, J=1.6 Hz, 1H), 5.81 (s, 1H), 3.90 (d, J=2.3 Hz, 1H), 3.42 (s, 2H).

### Example 38—2-methylene-4-oxo-4-((1-(4-(trifluoromethyl)phenyl)prop-2-yn-1-yl)oxy)butanoic acid (Enantiomer 2)

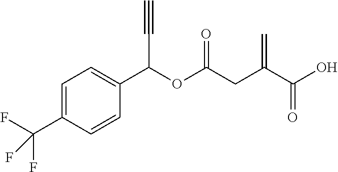 [SMILES: C#CC(OC(=O)CC(=C)C(=O)O)c1ccc(C(F)(F)F)cc1]

Prepared by an analogous method to Example 23 starting from 1-(4-(trifluoromethyl)phenyl)prop-2-yn-1-ol Enantiomer 2 (Intermediate 19, 220 mg, 1.10 mmol). Yield: 83 mg, 0.27 mmol. White solid. LCMS (System 2, Method B) m/z 334.9 (M+Na)⁺ (ES⁺). ¹H NMR (400 MHz, DMSO-d6) δ: 12.72 (br, 1H), 7.80 (d, J=8.2 Hz, 2H), 7.71 (d, J=8.2 Hz, 2H), 6.55 (d, J=2.3 Hz, 1H), 6.18 (d, J=1.6 Hz, 1H), 5.81 (d, J=1.4 Hz, 1H), 3.90 (d, J=2.3 Hz, 1H), 3.42 (s, 2H).

### Example 39—2-(1-((1-(4-(trifluoromethyl)phenyl)cyclobutoxy)carbonyl)cyclopropyl)acrylic acid

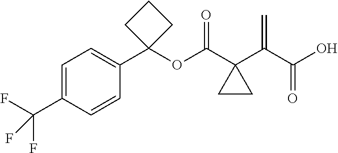 [SMILES: C=C(C(=O)O)C1(C(=O)OC2(c3ccc(C(F)(F)F)cc3)CCC2)CC1]

Prepared by an analogous method to Example 30 using 1-acetylcyclopropane-1-carboxylic acid (1.50 g, 11.7 mmol). Yield: 20 mg, 0.06 mmol. White solid. LCMS (System 2, Method B) m/z 376.9 (M+Na)⁺ (ES⁺). ¹H NMR (400 MHz, DMSO-d6) δ: 12.74 (br, 1H), 7.70 (d, J=8.3 Hz, 2H), 7.60 (d, J=8.2 Hz, 2H), 6.13 (s, 1H), 5.69 (s, 1H), 2.59-2.38 (m, 4H), 2.00-1.86 (m, 1H), 1.82-1.67 (m, 1H), 1.22 (q, J=4.1 Hz, 2H), 1.03 (q, J=4.2 Hz, 2H).

### Example 40—2-methylene-4-oxo-4-((3-(4-(trifluoromethyl)phenyl)thietan-3-yl)oxy)butanoic acid

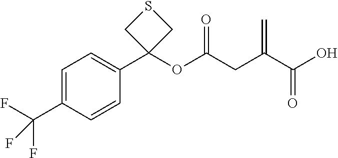 [SMILES: C=C(CC(=O)OC1(c2ccc(C(F)(F)F)cc2)CSC1)C(=O)O]

Prepared by an analogous method to Example 23 starting from 1 3-(4-(trifluoromethyl)phenyl)thietan-3-ol (Intermediate 20, 230 mg, 0.98 mmol). Yield: 184 mg, 0.53 mmol. White solid. LCMS (System 2, Method B) m/z 368.8 (M+Na)⁺ (ES⁺). ¹H NMR (400 MHz, DMSO-d6) δ: 12.77 (br, 1H), 7.86 (d, J=8.2 Hz, 2H), 7.78 (d, J=8.3 Hz, 2H), 6.15 (d, J=1.6 Hz, 1H), 5.77 (d, J=1.5 Hz, 1H), 3.87 (d, J=11.2 Hz, 2H), 3.52-3.45 (m, 2H), 3.37 (s, 2H).

### Example 41—4-((cis)-3-cyano-1-(4-(trifluoromethyl)phenyl)cyclobutoxy)-2-methylene-4-oxobutanoic acid

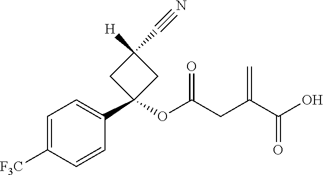 [SMILES: C=C(CC(=O)O[C@]1(c2ccc(C(F)(F)F)cc2)C[C@H](C#N)C1)C(=O)O]

Prepared by an analogous method to Example 23 starting from (cis)-3-hydroxy-3-(4-(trifluoromethyl)phenyl)cyclobutane-1-carbonitrile (Intermediate 21, 240 mg, 1.00 mmol). Yield: 120 mg, 0.34 mmol. White solid. LCMS (System 2, Method B) m/z 375.9 (M+Na)⁺ (ES⁺). ¹H NMR (400 MHz, DMSO-d6) δ: 12.74 (br, 1H), 7.73 (d, J=8.3 Hz, 2H), 7.66 (d, J=8.3 Hz, 2H), 6.14 (d, J=1.6 Hz, 1H), 5.76 (s, 1H), 3.36 (s, 2H), 3.29 (q, J=8.7 Hz, 1H), 3.13-3.02 (m, 2H), 2.89-2.78 (m, 2H).

### Example 42—4-((cis)-3-methoxy-1-(4-(trifluoromethyl)phenyl)cyclobutoxy)-2-methylene-4-oxobutanoic acid

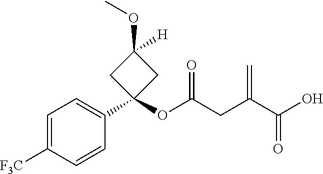 [SMILES: C=C(CC(=O)O[C@]1(c2ccc(C(F)(F)F)cc2)C[C@H](OC)C1)C(=O)O]

Prepared by an analogous method to Example 23 starting from (cis)-3-methoxy-1-(4-(trifluoromethyl)phenyl)cyclobutan-1-ol (Intermediate 22, 320 mg, 1.30 mmol). Yield: 154 mg, 0.43 mmol. White solid. LCMS (System 2, Method B) m/z 380.9 (M+Na)⁺ (ES⁺). ¹H NMR (400 MHz, DMSO-d6) δ: 12.70 (br, 1H), 7.70 (d, J=8.4 Hz, 2H), 7.61 (d, J=8.4 Hz, 2H), 6.14 (d, J=1.2 Hz, 1H), 5.75 (d, J=1.2 Hz, 1H), 3.80 (t, J=6.8 Hz, 1H), 3.34 (s, 2H), 3.17 (s, 3H), 3.01-2.96 (m, 2H), 2.42-2.36 (m, 2H).

### Example 43—2-methylene-4-oxo-4-(3-(trifluoromethyl)-1-(4-(trifluoromethyl)phenyl)cyclobutoxy)butanoic acid

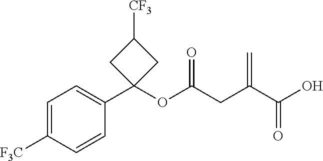 [SMILES: C=C(CC(=O)OC1(c2ccc(C(F)(F)F)cc2)CC(C(F)(F)F)C1)C(=O)O]

Prepared by an analogous method to Example 23 starting from 3-(trifluoromethyl)-1-(4-(trifluoromethyl)phenyl)cyclobutanol (Intermediate 23, 250 mg, 0.88 mmol). Yield: 83 mg, 0.21 mmol (cis isomer 96%/trans isomer 4%). White solid. LCMS (System 2, Method B) m/z 397.0 (M+H)⁺ (ES⁺). ¹H NMR (400 MHz, DMSO-d6) δ: 12.65 (br, 1H), 7.77-7.68 (m, 4H), 6.14 (s, 1H), 5.75 (s, 1H), 3.35 (s, 2H), 3.25-3.10 (m, 1H), 2.92-2.86 (m, 2H), 2.67-2.57 (m, 2H).

### Example 44—4-(3-methyl-1-(4-(trifluoromethyl)phenyl)cyclobutoxy)-2-methylene-4-oxobutanoic acid

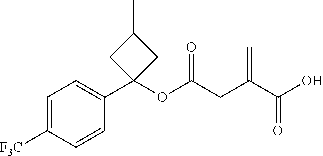 [SMILES: C=C(CC(=O)OC1(c2ccc(C(F)(F)F)cc2)CC(C)C1)C(=O)O]

Prepared by an analogous method to Example 23 starting from 3-methyl-1-(4-(trifluoromethyl)phenyl)cyclobutan-1-ol (Intermediate 24, 228 mg, 0.87 mmol). Yield: 105 mg, 0.30 mmol (cis isomer 93%/trans isomer 7%). White solid. LCMS (System 2, Method B) m/z 365.0 (M+Na)⁺ (ES⁺). ¹H NMR (400 MHz, DMSO-d6) δ: 12.67 (s, 1H), 7.67 (d, J=8.4 Hz, 2H), 7.62 (d, J=8.4 Hz, 2H), 6.06 (s, 1H), 5.64 (s, 1H), 3.31 (s, 2H), 2.73-2.67 (m, 2H), 2.14-2.04 (m, 3H), 1.11 (d, J=5.6 Hz, 3H).

### Example 45—(cis)-4-(3-methyl-1-(4-(trifluoromethyl)phenyl)cyclobutoxy)-2-methylene-4-oxobutanoic acid

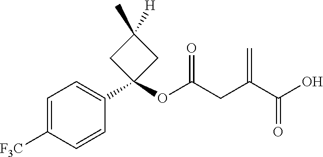 [SMILES: C=C(CC(=O)O[C@]1(c2ccc(C(F)(F)F)cc2)C[C@H](C)C1)C(=O)O]

Prepared by an analogous method to Example 23 starting from (cis)-3-methyl-1-(4-(trifluoromethyl)phenyl)cyclobutan-1-ol (Intermediate 25, 284 mg, 1.09 mmol). Yield: 200 mg, 64%. White solid. LCMS (System 2, Method B) m/z 365.9 (M+Na)⁺ (ES⁺). ¹H NMR (400 MHz, DMSO-d6) δ: 12.68 (s, 1H), 7.70 (d, J=8.4 Hz, 2H), 7.64 (d, J=8.4 Hz, 2H), 6.13 (s, 1H), 5.73 (s, 1H), 3.30 (s, 2H), 2.77-2.74 (m, 2H), 2.16-2.07 (m, 3H), 1.14 (d, J=5.6 Hz, 3H).

### Example 46—2-methylene-4-oxo-4-(3-(5-(trifluoromethyl)pyridin-2-yl)oxetan-3-yloxy)butanoic acid

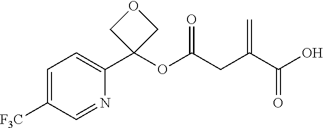 [SMILES: C=C(CC(=O)OC1(c2ccc(C(F)(F)F)cn2)COC1)C(=O)O]

Prepared by an analogous method to Example 23 starting from 3-(5-(trifluoromethyl)pyridin-2-yl)oxetan-3-ol (Intermediate 26, 300 mg, 1.37 mmol). Yield: 155 mg, 65%. White solid. LCMS (System 2, Method B) m/z 331.9 (M+H)⁺ (ES⁺). ¹H NMR (400 MHz, DMSO-d6) δ: 12.83 (br, 1H), 9.08 (dt, J=2.1, 1.0 Hz, 1H), 8.22 (dd, J=8.5, 2.4, Hz, 1H), 7.68 (d, J=8.4 Hz, 1H), 6.21 (d, J=1.2 Hz, 1H), 5.86 (d, J=1.2 Hz, 1H), 4.99 (d, J=7.6 Hz, 2H), 4.84 (d, J=7.6 Hz, 2H), 3.56 (s, 2H).

### Example 47—2-methylene-4-oxo-4-((4-(4-(trifluoromethyl)phenyl)tetrahydro-2H-pyran-4-yl)oxy)butanoic acid

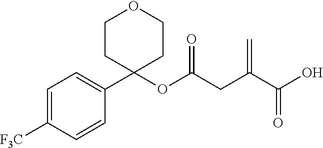 [SMILES: C=C(CC(=O)OC1(c2ccc(C(F)(F)F)cc2)CCOCC1)C(=O)O]

Prepared by an analogous method to Example 2 starting from 4-(4-(trifluoromethyl)phenyl)tetrahydro-2H-pyran-4-ol (Intermediate 27). Yield: 86 mg. White solid. LCMS (System 2, Method B) m/z 380.9 (M+Na)⁺ (ES⁺). ¹H NMR (400 MHz, DMSO-d6) δ: 12.74 (br, 1H), 7.67 (d, J=8.4 Hz, 2H), 7.56 (d, J=8.4 Hz, 2H), 6.13 (s, 1H), 5.77 (s, 1H), 3.78-3.74 (m, 2H), 3.68-3.63 (m, 2H), 3.38 (s, 2H), 2.20-2.16 (m, 2H), 2.07-1.98 (m, 2H).

### Example 48—4-((1-methyl-3-(4-(trifluoromethyl)phenyl)azetidin-3-yl)oxy)-2-methylene-4-oxobutanoic acid

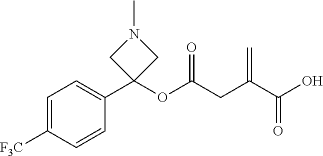 [SMILES: C=C(CC(=O)OC1(c2ccc(C(F)(F)F)cc2)CN(C)C1)C(=O)O]

Prepared by an analogous method to Example 23 starting from 1-methyl-3-(4-(trifluoromethyl)phenyl)azetidin-3-ol (Intermediate 29, 200 mg, 0.86 mmol). The crude compound was purified by prep-HPLC (Column: Waters X-Bridge C18 OBD 10 μm 19×250 mm; Flow Rate: 20 mL/min; solvent system: MeCN/(0.2% TFA/water) gradient: MeCN: 30-65%; collection wavelength: 214 nm). The fractions were concentrated under reduced pressure to remove MeCN, and the residue was lyophilized to give 4-((1-methyl-3-(4-(trifluoromethyl)phenyl)azetidin-3-yl)oxy)-2-methylene-4-oxobutanoic acid (TFA salt, 75 mg, 35% yield) as white solid. LCMS (System 2, Method B) m/z 344.0 (M+H)⁺ (ES⁺). ¹H NMR (400 MHz, DMSO-d6) δ: 12.85 (br, 1H), 10.97-10.54 (br, 1H), 7.82-7.74 (m, 4H), 6.18 (s, 1H), 5.79 (s, 1H), 4.84-4.50 (m, 4H), 3.44 (s, 2H), 2.96 (br, 3H).

### Example 49—4-((1,1-dioxido-3-(4-(trifluoromethyl)phenyl)thietan-3-yl)oxy)-2-methylene-4-oxobutanoic acid

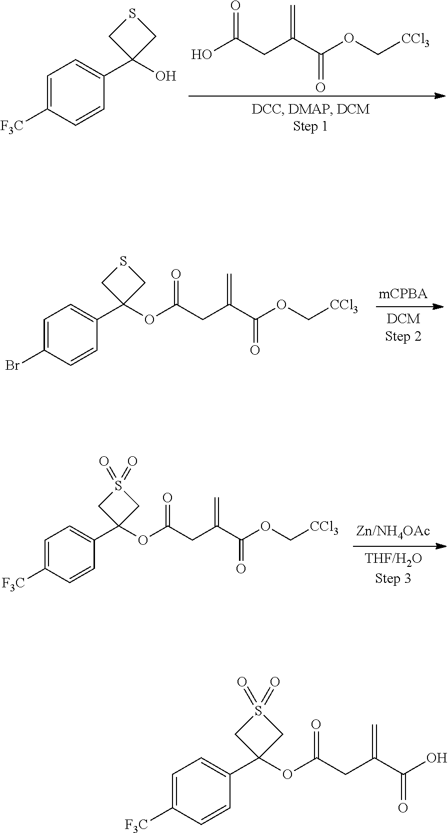

### Step 1

A mixture of 3-(4-(trifluoromethyl)phenyl)thietan-3-ol (Intermediate 20, 230 mg, 0.98 mmol) 3-((2,2,2-trichloroethoxy)carbonyl)but-3-enoic acid (257 mg, 0.98 mmol), DCC (303 mg, 1.47 mmol) and DMAP (13 mg, 0.10 mmol) in DCM (2 mL) was stirred at room temperature for 30 minutes. The mixture was filtered, and the filtrate was concentrated under reduced pressure. The residue was purified by flash column chromatography (0-15% MTBE/petroleum ether) to give 1-(2,2,2-trichloroethyl) 4-(3-(4-(trifluoromethyl)phenyl)thietan-3-yl) 2-methylenesuccinate (400 mg, 85% yield) as a colorless oil. LCMS (System 2, Method C) m/z 499 (M+Na)⁺ (ES⁺).

### Step 2

A mixture of 1-(2,2,2-trichloroethyl) 4-(3-(4-(trifluoromethyl)phenyl)thietan-3-yl) 2-methylenesuccinate (400 mg, 0.84 mmol) and m-CPBA (375 mg, 1.85 mmol, 85 wt %) in DCM (5 mL) was stirred at room temperature for 2 hrs. The mixture was quenched with saturated sodium bicarbonate (5 mL) and extracted with DCM (3×5 mL). The combined organic layers were washed with brine, dried over Na₂SO₄ and concentrated under reduced pressure. The residue was purified by flash column chromatography (0-40% ethyl acetate/petroleum ether) to give 4-(1,1-dioxido-3-(4-(trifluoromethyl)phenyl)thietan-3-yl) 1-(2,2,2-trichloroethyl) 2-methylenesuccinate (280 mg, 66% yield) as a colorless oil. LCMS (System 2, Method C) m/z 530.7 (M+Na)⁺ (ES⁺).

### Step 3

Prepared by an analogous method to Example 23/Step 2. Yield: 97 mg, 47%. White solid. LCMS (System 2, Method B) m/z 400.9 (M+Na)⁺ (ES⁺). ¹H NMR (400 MHz, DMSO-d6) δ: 12.73 (br, 1H), 7.79-7.73 (m, 4H), 6.17 (s, 1H), 5.80 (s, 1H), 5.04 (d, J=15.6 Hz, 2H), 4.78 (d, J=15.6 Hz, 2H), 3.44 (s, 2H).

### Example 50—4-(1-(5-bromopyridin-2-yl)cyclobutoxy)-2-methylene-4-oxobutanoic acid

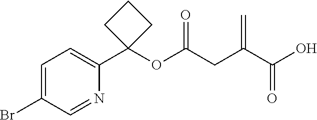 [SMILES: C=C(CC(=O)OC1(c2ccc(Br)cn2)CCC1)C(=O)O]

Prepared by an analogous method to Example 23 starting from 1-(5-bromopyridin-2-yl)cyclobutanol (Intermediate 30, 300 mg, 1.32 mmol). Yield: 73 mg, 42%. Light yellow solid. LCMS (System 2, Method B) m/z 339.9 (M+H)⁺ (ES⁺). ¹H NMR (400 MHz, DMSO-d6) δ: 12.72 (br, 1H), 8.69 (d, J=2 Hz, 1H), 7.98 (dd, J=8.5, 2.4H, 1H), 7.35 (d, J=8.4 Hz, 1H), 6.15 (d, J=1.2 Hz, 1H), 5.78 (d, J=1.2 Hz, 1H), 3.38 (s, 2H), 2.67-2.60 (m, 2H), 2.50-2.39 (m, 2H), 1.94-1.84 (m, 2H).

### Example 51—4-(1-(5-chloropyridin-2-yl)cyclobutoxy)-2-methylene-4-oxobutanoic acid

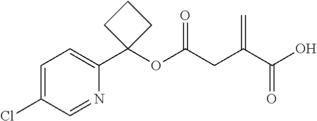 [SMILES: C=C(CC(=O)OC1(c2ccc(Cl)cn2)CCC1)C(=O)O]

Prepared by an analogous method to Example 23 starting from 1-(5-chloropyridin-2-yl)cyclobutan-1-ol (Intermediate 31, 220 mg, 1.20 mmol Yield: 70.80 mg, 53%. White solid. LCMS (System 2, Method B) m/z 296.0 (M+H)⁺ (ES⁺). ¹H NMR (400 MHz, DMSO-d6) δ: 12.72 (s, 1H), 8.61 (dd, J=2.8, 0.8 Hz, 1H), 7.86 (dd, J=8.4, 2.4 Hz, 1H), 7.40 (dd, J=8.4, 0.8 Hz, 1H), 6.15 (d, J=1.6 Hz, 1H), 5.78 (d, J=1.2 Hz, 1H), 3.37 (s, 2H), 2.67-2.60 (m, 2H), 2.49-2.32 (m, 2H), 1.95-1.82 (m, 2H).

### Example 52—2-methylene-4-oxo-4-(1-(5-(trifluoromethyl)pyrimidin-2-yl)cyclobutoxy)butanoic acid

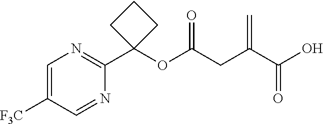 [SMILES: C=C(CC(=O)OC1(c2ncc(C(F)(F)F)cn2)CCC1)C(=O)O]

Prepared by an analogous method to Example 23 starting from 1-(5-(trifluoromethyl)pyrimidin-2-yl)cyclobutan-1-ol (Intermediate 32, 170 mg, 0.78 mmo). Yield 13.5 mg, 15%. White solid. LCMS (System 2, Method B) m/z 331.0 (M+H)⁺ (ES⁺). ¹H NMR (400 MHz, DMSO-d6) δ: 12.66 (s, 1H), 9.27 (d, J=0.8 Hz, 2H), 6.15 (d, J=1.2 Hz, 1H), 5.81 (d, J=0.8 Hz, 1H), 3.36 (s, 2H), 2.74-2.66 (m, 2H), 2.52-2.42 (m, 2H), 2.02-1.98 (m, 2H).

### Example 53—4-(1-(3,5-difluoro-4-(trifluoromethyl)phenyl)cyclobutoxy)-2-methylene-4-oxobutanoic acid

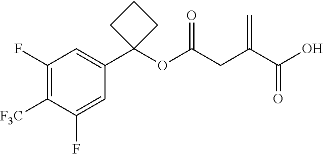 [SMILES: C=C(CC(=O)OC1(c2cc(F)c(C(F)(F)F)c(F)c2)CCC1)C(=O)O]

Prepared by an analogous method to Example 2 starting from 1-(3,5-difluoro-4-(trifluoromethyl)phenyl)cyclobutanol (Intermediate 33, 4 g, 15.8 mmol). Yield: 115 mg, 0.32 mmol. White solid. LCMS (System 2, Method B) m/z 386.8 (M+Na)⁺ (ES⁺). ¹H NMR (400 MHz, DMSO-d6) δ: 12.74 (br, 1H), 7.42 (d, J=11.2 Hz, 2H), 6.15 (s, 1H), 5.78 (s, 1H), 3.37 (s, 2H), 2.59-2.53 (m, 2H), 2.46-2.44 (m, 2H), 1.97-1.91 (m, 1H), 1.87-1.79 (m, 1H).

### Example 54—4-(1-(6-bromonaphthalen-2-yl)cyclobutoxy)-2-methylene-4-oxobutanoic acid

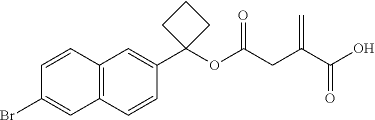 [SMILES: C=C(CC(=O)OC1(c2ccc3cc(Br)ccc3c2)CCC1)C(=O)O]

Prepared by an analogous method to Example 23 starting from 1-(6-bromonaphthalen-2-yl)cyclobutan-1-ol (Intermediate 34,189 mg, 0.72 mmol). Yield: 139 mg, 62%. White solid. LCMS (System 2, Method B) m/z 410.8 (M+Na)⁺ (ES⁺). ¹H NMR (400 MHz, DMSO-d6) δ: 12.65 (s, 1H), 8.19 (d, J=1.6 Hz, 1H), 8.02 (s, 1H), 7.92 (dd, J=14.8, 8.8 Hz, 2H), 7.68-7.56 (m, 2H), 6.12 (d, J=1.2 Hz, 1H), 5.72 (d, J=1.6 Hz, 1H), 3.33 (s, 2H), 2.69-2.62 (m, 2H), 2.60-2.52 (m, 2H), 2.00-1.94 (m, 1H), 1.83-1.76 (m, 1H).

### Example 55—4-((3-fluoro-4-(trifluoromethyl)benzyl)oxy)-2-methylene-4-oxobutanoic acid

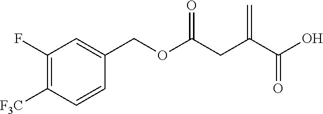 [SMILES: C=C(CC(=O)OCc1ccc(C(F)(F)F)c(F)c1)C(=O)O]

Prepared by an analogous method to Example 23 starting from (3-fluoro-4-(trifluoromethyl)phenyl)methanol (260 mg, 1.00 mmol). Yield: 144.10 mg, 51%. White solid. LCMS (System 2, Method B) m/z 307.0 (M+Na)⁺ (ES⁺). ¹H NMR (400 MHz, DMSO-d6) δ: 12.78 (br, 1H), 7.79 (t, J=8.0 Hz, 1H), 7.48 (d, J=12.0 Hz, 1H), 7.39 (d, J=8.0 Hz, 1H), 6.18 (s, 1H), 5.81 (s, 1H), 5.22 (s, 2H), 3.44 (s, 2H).

### Example 56—2-methylene-4-oxo-4-((5-(trifluoromethyl)pyridin-2-yl)methoxy)butanoic acid

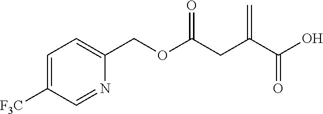 [SMILES: C=C(CC(=O)OCc1ccc(C(F)(F)F)cn1)C(=O)O]

Prepared by an analogous method to Example 23 starting from (5-(trifluoromethyl)pyridin-2-yl)methanol (180 mg, 1.02 mmol). Yield: 142.11 mg, 52%. White solid. LCMS (System 2, Method B) m/z 290.0 (M+H)⁺ (ES⁺). ¹H NMR (400 MHz, DMSO-d6) δ: 12.73 (s, 1H), 8.95 (s, 1H), 8.24 (dd, J=8.0, 2.0 Hz, 1H), 7.62 (d, J=8.4 Hz, 1H), 6.20 (d, J=1.6 Hz, 1H), 5.84 (d, J=0.8 Hz, 1H), 5.27 (s, 2H), 3.47 (s, 2H).

### Example 57—2-methylene-4-oxo-4-((2-(5-(trifluoromethyl)pyridin-2-yl)propan-2-yl)oxy)butanoic acid

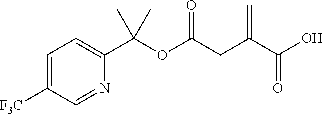 [SMILES: C=C(CC(=O)OC(C)(C)c1ccc(C(F)(F)F)cn1)C(=O)O]

Prepared by an analogous method to Example 2 starting from 2-(5-(trifluoromethyl)pyridin-2-yl)propan-2-ol (Intermediate 35, 650 mg, 3.17 mmol), Yield: 65 mg, 0.20 mmol. Colourless oil. LCMS (System 2, Method B) m/z 318.0 (M+H)⁺ (ES⁺). ¹H NMR (400 MHz, DMSO-d6) δ: 12.70 (br, 1H), 8.90 (s, 1H), 8.15 (dd, J=8.4, 2.0 Hz, 1H), 7.65 (d, J=8.4 Hz, 1H), 6.14 (s, 1H), 5.77 (s, 1H), 3.36 (s, 2H), 1.69 (s, 6H).

### Example 58—2-methylene-4-oxo-4-(1-(5-(trifluoromethyl)pyridin-2-yl)cyclopropoxy)butanoic acid

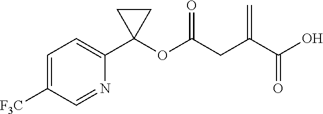 [SMILES: C=C(CC(=O)OC1(c2ccc(C(F)(F)F)cn2)CC1)C(=O)O]

Prepared by an analogous method to Example 23 starting from 1-(5-(trifluoromethyl)pyridin-2-yl)cyclopropanol (Intermediate 36, 150 mg, 0.74 mmol). Yield: 89 mg, 50%. White solid. LCMS (System 2, Method B) m/z 316.0 (M+H)⁺ (ES⁺). ¹H NMR (400 MHz, DMSO-d6) δ: 12.82 (s, 1H), 8.85 (s, 1H), 8.09 (dd, J=8.4, 1.6 Hz, 1H), 7.61 (d, J=8.4 Hz, 1H), 6.20 (s, 1H), 5.84 (s, 1H), 3.48 (s, 2H), 1.59-1.55 (m, 2H), 1.39-1.35 (m, 2H).

### Example 59—3,3-dimethyl-2-methylene-4-oxo-4-(1-(5-(trifluoromethyl)pyridin-2-yl)cyclobutoxy)butanoic acid

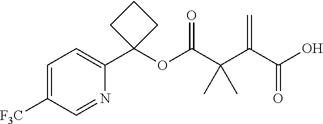 [SMILES: C=C(C(=O)O)C(C)(C)C(=O)OC1(c2ccc(C(F)(F)F)cn2)CCC1]

Prepared by an analogous method to Example 30 starting from 1-(5-(trifluoromethyl)pyridin-2-yl)cyclobutan-1-ol (Intermediate 9, 1.06 g, 4.90 mmol). Yield: 127.5 mg, 0.36 mmol. White solid. LCMS (System 2, Method B) m/z 358.0 (M+H)⁺ (ES⁺). ¹H NMR (400 MHz, DMSO-d6) δ: 12.85 (s, 1H), 8.97 (d, J=0.4 Hz, 1H), 8.14 (dd, J=8.4, 2.0 Hz, 1H), 7.51 (d, J=8.4 Hz, 1H), 6.24 (s, 1H), 5.83 (s, 1H), 2.68-2.61 (m, 2H), 2.45-2.37 (m, 2H), 1.93-1.86 (m, 2H), 1.33 (s, 6H).

### Example 60—2-methylene-4-oxo-4-(1-((4-(trifluoromethyl)phenyl)ethynyl) cyclobutoxy)butanoic acid

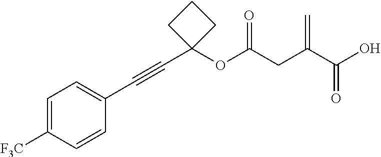 [SMILES: C=C(CC(=O)OC1(C#Cc2ccc(C(F)(F)F)cc2)CCC1)C(=O)O]

Prepared by an analogous method to Example 2 starting from 1-((4-(trifluoromethyl)phenyl)ethynyl)cyclobutanol (Intermediate 37, 5.00 g, 20.81 mmol). Yield: 57 mg, 0.16 mmol. White solid. LCMS (System 2, Method B) m/z 375.0 (M+Na)⁺ (ES⁺). ¹H NMR (400 MHz, DMSO-d6) δ: 12.70 (s, 1H), 7.75 (d, J=8.0 Hz, 2H), 7.64 (d, J=8.0 Hz, 2H), 6.17 (s, 1H), 5.80 (s, 1H), 3.35 (s, 2H), 2.59-2.54 (m, 2H), 2.45-2.42 (m, 2H), 1.96-1.92 (m, 2H).

### Example 61—2-methylene-4-oxo-4-((5-(4-(trifluoromethyl)phenyl)spiro[2.3]hexan-5-yl)oxy)butanoic acid

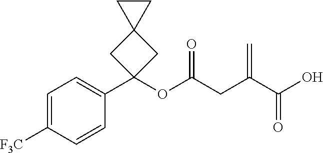 [SMILES: C=C(CC(=O)OC1(c2ccc(C(F)(F)F)cc2)CC2(CC2)C1)C(=O)O]

Prepared by an analogous method to Example 23 starting from 5-(4-(trifluoromethyl)phenyl)spiro[2.3]hexan-5-ol (Intermediate 28, 216 mg, 0.83 mmol). Yield: 76 mg, 36%. White solid. LCMS (System 2, Method B) m/z 376.9 (M+Na)⁺ (ES⁺). ¹H NMR (400 MHz, DMSO-d6) δ: 12.69 (s, 1H), 7.71-7.66 (m, 4H), 6.13 (d, J=1.2 Hz, 1H), 5.75 (d, J=1.2 Hz, 1H), 3.36 (s, 2H), 2.78-2.74 (m, 2H), 2.50-2.48 (m, 2H), 0.56-0.49 (m, 4H).

### Example 62—2-methylene-4-oxo-4-(1-(6-(trifluoromethyl)pyridazin-3-yl)cyclobutoxy)butanoic acid

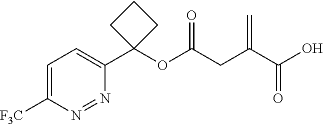 [SMILES: C=C(CC(=O)OC1(c2ccc(C(F)(F)F)nn2)CCC1)C(=O)O]

Prepared by an analogous method to Example 23 starting from 1-(6-(trifluoromethyl)pyridazin-3-yl)cyclobutan-1-ol (Intermediate 38, 260 mg, 1.19 mmol). Yield: 18 mg, 14%. White solid. LCMS (System 2, Method C) m/z 331.1 (M+H)⁺ (ES⁺). ¹H NMR (400 MHz, DMSO-d6) δ: 7.82-7.71 (m, 2H), 6.47 (s, 1H), 5.89 (s, 1H), 3.42 (s, 2H), 2.94-2.89 (m, 2H), 2.67-2.64 (m, 2H), 2.15-2.11 (m, 2H).

### Example 63—2-methylene-4-oxo-4-(1-(5-(trifluoromethyl)pyrazin-2-yl)cyclobutoxy)butanoic acid

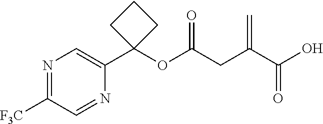 [SMILES: C=C(CC(=O)OC1(c2cnc(C(F)(F)F)cn2)CCC1)C(=O)O]

Prepared by an analogous method to Example 23 starting from 1-(5-(trifluoromethyl)pyrazin-2-yl)cyclobutan-1-ol (Intermediate 39, 230 mg, 1.05 mmol). Yield: 50 mg, 22%. White solid. LCMS (System 2, Method B) m/z 331.1 (M+H)⁺ (ES⁺). ¹H NMR (400 MHz, DMSO-d6) δ: 12.79 (br, 1H), 9.20 (s, 1H), 8.87 (s, 1H), 6.16 (s, 1H), 5.81 (s, 1H), 3.44 (s, 2H), 2.76-2.69 (m, 2H), 2.55-2.52 (m, 2H), 2.02-1.89 (m, 2H).

### Biological Example 1—THP-1 AlphaLISA IL-1p and IL-6 Cytokine Assay

Measuring Inhibitory Effects on IL-1V and IL-6 Cytokine Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β and IL-6 cytokine inhibition assays were run in a background of differentiated THP-1 cells as described below. All reagents described were from Sigma-Aldrich unless specified otherwise. Compounds were prepared as 10 mM DMSO stocks.

Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the THP-1s were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from each of the IL-1β and IL-6 commercial kits (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalising the sample data to the high and low controls used within each plate (+/−LPS respectively). Percentage inhibition was then plotted against compound concentration and the 50% inhibitory concentration (IL₅₀) was determined from the resultant concentration-response curve.

Compounds of formula (I) were tested in this assay and the results of those compounds tested are shown in Table 1 below. Dimethyl itaconate, dimethyl fumarate and 4-octyl itaconate were included as comparator compounds.

TABLE 1
THP-1 cell IL-1β and IL-6 IC₅₀ values (μM)

| Compound | IL-1β (IC₅₀) | IL-6 (IC₅₀) |
|---|---|---|
| dimethyl fumarate | 14.4 | 9.3 |
| dimethyl itaconate | >100 | 100 |
| 4-octyl itaconate | 38.3 | 20.3 |
| Example 1 | 0.8 | 62 |
| Example 2 | 0.8 | >63.1 |
| Example 3 | 33.6 | >56.2 |
| Example 4 | 1.9 | >56.2 |
| Example 5 | 0.7 | >31.6 |
| Example 6 | 45.4 | >100 |
| Example 7 | 3.7 | >100 |
| Example 8 | 2.6 | 64.5 |
| Example 9 | 2.8 | >100 |
| Example 10 | 5.8 | >100 |
| Example 11 | 1.4 | >100 |
| Example 12 | 4.5 | 82.6 |
| Example 13 | 5.9 | >100 |
| Example 14 | 24.5 | >100 |
| Example 15 | 15.9 | >100 |
| Example 16 | >100 | 21.5 |
| Example 17 | 2.0 | >31.6 |
| Example 18 | 1.8 | 62.1 |
| Example 19 | 1.2 | >68.1 |
| Example 20 | 3.0 | 48.7 |
| Example 21 | 7.3 | 49.7 |
| Example 22 | 5.1 | >100 |
| dimethyl fumarate | 14.4 | 9.3 |
| dimethyl itaconate | >100 | 100 |
| 4-octyl itaconate | 38.3 | 20.3 |
| Example 23 | 1.4 | >100 |
| Example 24 | 1.4 | >100 |
| Example 25 | 15.5 | >100 |
| Example 26 | 42 | 3.7 |
| Example 27 | 1.4 | >100 |
| | 4.0^{a} | |
| Example 28 | >31.6 | 19.6 |
| Example 29 | 1.3 | >100 |
| Example 30 | 1.2 | >100 |
| Example 31 | 3.4 | >100 |
| Example 32 | 5.2 | >100 |
| Example 33 | >56.2 | >63.1 |
| Example 34 | 12.2 | >31.6 |
| Example 35 | 8.8 | >31.6 |
| Example 36 | >31.6 | 5.6 |
| Example 37 | 14.5 | >31.6 |
| Example 38 | 9.4 | >100 |
| Example 39 | 1.5 | >100 |
| Example 40 | 0.8 | >100 |
| Example 41 | 2.1 | >31.6 |
| Example 42 | 17.1 | >31.6 |
| Example 43 | 83.5 | >100 |
| Example 44 | 5.1 | >100 |
| Example 45 | 3.4 | >100 |
| Example 46 | 4.1 | >31.6 |
| Example 47 | 6.7 | >100 |
| Example 48 | 58.4 | >31.6 |
| Example 49 | 5.0 | >100 |
| Example 50 | 1.5 | >100 |
| Example 51 | 6.4 | >31.6 |
| Example 52 | 11.7 | >100 |
| Example 53 | 29.7 | >100 |
| dimethyl fumarate | 14.4 | 9.3 |
| dimethyl itaconate | >100 | 100 |
| 4-octyl itaconate | 38.3 | 20.3 |
| Example 54 | >100 | 18.1 |
| Example 55 | 19.5 | >100 |
| Example 56 | 46.2 | >100 |
| Example 57 | 6.6 | >31.6 |
| Example 58 | 3.2 | >42.2 |
| Example 59 | 2.9 | >31.6 |
| Example 60 | >100 | 34.1 |
| Example 61 | 5.5 | >100 |
| Example 62 | 5 | >31.6 |
| Example 63 | 50.9 | >31.6 |

NT* = not tested;
^{a}data from repeated experiments

Compounds of formula (I) that were tested in this assay exhibited improved cytokine-lowering potencies compared to dimethyl itaconate in IL-1p3 and/or IL-6. Preferred compounds that were tested in this assay exhibited improved cytokine-lowering potencies compared to dimethyl fumarate and/or 4-octyl itaconate in IL-1p3 and/or IL-6. For example, Example 1 exhibited much improved cytokine-lowering potencies compared to dimethyl itaconate, dimethyl fumarate and 4-octyl itaconate for IL-1p. Example 1 exhibited improved cytokine-lowering potencies compared to dimethyl itaconate for IL-6. Comparison of the IL-1p3 IC₅₀ data for Examples 33 and 34 indicates that there can be a stereochemical preference for substituents with the absolute configuration of Example 34 on the benzylic position.

### Biological Example 2—NRF2+/− GSH Activation Assay

Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

The assay was run under either +/− GSH (glutathione) conditions to determine the attenuating activities of GSH against target compounds.

Additionally, a defined concentration of dimethyl fumarate was used as the ‘High’ control to normalise test compound activation responses to.

Assay Procedure

U2OS PathHunter eXpress cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound, for −GSH conditions, or for + GSH conditions, an intermediate plate containing 6×working concentrations of compound stocks was prepared in a 6 mM working concentration of GSH solution (solubilised in sterile PBS). Following a 30 minute compound-GSH pre-incubation (37° C./5% CO₂) for +GSH treatment, plated U2OS cells were incubated with an appropriate final concentration of compound and GSH.

Following compound (+/−GSH) treatment, the U2OS plates were incubated for a further 6 hours (37° C./5% CO₂) before detection reagent from the PathHunter NRF2 commercial kit was prepared and added to test plates according to the manufacturer's instructions. Subsequently, the luminescence signal detection in a microplate reader was measured (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

A number of compounds of formula (I) were tested in this assay, and the results are shown in Table 2 below. Dimethyl itaconate, dimethyl fumarate and 4-octyl itaconate were included as comparator compounds.

TABLE 2
NRF2 activation

| | −GSH | | +GSH | |
|---|---|---|---|---|
| Compound | EC₅₀ (μM) | Eₘₐₓ (%) | EC₅₀ (μM) | Eₘₐₓ (%) |
| dimethyl fumarate | 6.0 | 100 | >100 | 19 |
| dimethyl itaconate | 21.4 | 137 | 56.4 | 77 |
| 4-octyl itaconate | 22.1 | 165 | 28.9 | 148 |
| Example 1 | >100 | 37 | >100 | 10 |
| | | 17^{a} | | 14^{a} |
| Example 2 | >100 | 0 | >100 | 0 |
| Example 4 | >100 | 4 | >100 | 0 |
| Example 7 | >100 | 6 | >100 | 0 |
| Example 8 | >100 | 24 | >100 | 0 |
| Example 9 | >100 | 14 | >100 | 6 |
| Example 10 | >100 | 0 | >100 | 13 |
| Example 11 | >100 | 11 | >100 | 0 |
| Example 12 | 81.6 | 66 | >100 | 46 |
| Example 13 | 38.7 | 54 | >100 | 25 |
| Example 21 | >100 | 0 | >100 | 4 |
| Example 26 | 24.1 | 88 | >100 | 34 |
| Example 36 | 9.0 | 116 | 8.6 | 90 |
| Example 40 | 1.7 | 61 | >100 | 9 |
| Example 58 | >100 | 1 | >100 | 0 |
| Example 62 | >100 | 1 | >100 | 4 |

^{a}data from repeated experiments

Many of the compounds of formula (I) tested in this assay showed very little activity in this assay, as demonstrated by their EC₅₀ and/or Eₘₐₓ, values for NRF2 activation, indicating that the IL1β-lowering effect is not a consequence of NRF2 activation.

### Biological Example 3—Hepatocyte Stability Assay

Defrosted cryo-preserved hepatocytes (viability>70%) were used to determine the metabolic stability of a compound via calculation of intrinsic clearance (Clᵢₙₜ; a measure of the removal of a compound from the liver in the absence of blood flow and cell binding). Clearance data are particularly important for in vitro work as they can be used in combination with in vivo data to predict the half-life and oral bioavailability of a drug.

The metabolic stability in hepatocytes assay involved a time-dependent reaction using both positive and negative controls. The cells must be pre-incubated at 37° C. then spiked with test compound (and positive control); samples taken at pre-determined time intervals were analysed to monitor the change in concentration of the initial drug compound over 60 minutes. A buffer incubation reaction (with no hepatocytes present) acted as a negative control and two cocktail solutions, containing compounds with known high and low clearance values (verapamil/7-hydroxycoumarin and propranolol/diltiazem), acted as positive controls.
1. The assay was run with a cell concentration of 0.5×10⁶ cells/mL in Leibovitz buffer.
2. All compounds and controls were run in duplicate.
3. Compound concentration was 10 μM.
4. All compounds and controls were incubated with both cells and buffer to show turnover is due to hepatic metabolism.
5. All wells on the incubation plate had 326.7 μL of either cells or buffer added.
6. Prior to assay, cell and buffer-only incubation plates were preincubated for 10 mins at 37° C.
7. The assay was initiated by adding compounds, 3.3 μL of 1 mM in 10% DMSO-90% Buffer; final DMSO concentration is 0.1%.
8. Samples were taken at regular timepoints (0, 5, 10, 20, 40, 60 min) until 60 mins.
9. Sample volume was 40 μL and added to 160 μL of crash solvent (acetonitrile with internal standard) and stored on ice.
10. At the end of the assay, the crash plates were centrifuged at 3500 rpm for 20 mins at 4° C.
11. 80 μL of clear supernatant was removed and mixed with 80 μL of deionised water before being analysed by LC-MS/MS.

Raw LC-MS/MS data were exported to, and analysed in, Microsoft Excel for determination of intrinsic clearance. The percentage remaining of a compound was monitored using the peak area of the initial concentration as 100%. Intrinsic clearance and half-life values were calculated using a graph of the natural log of percentage remaining versus the time of reaction in minutes. Half-life (min) and intrinsic clearance (Cliₙₜ in μL min⁻¹ 10⁻⁶ cells) values were calculated using the gradient of the graph (the elimination rate constant, k) and Equations 1 and 2.

$$\begin{matrix}{t}_{\frac{1}{2}} = \frac{ln 2}{k} & \{ Equation 1 \} \\ {Cl}_{int} = ( \frac{ln 2}{{t}_{\frac{1}{2}}} ) \times ( \frac{350}{0.175} ) & \{ Equation 2 \}\end{matrix}$$

A number of compounds of formula (I) were tested in this assay, and the results are shown in Table 3 below. 4-Octyl itaconate was included as a comparator compound.

TABLE 3
Hepatocyte stability

| Compound | Species | Clᵢₙₜ (μL min⁻¹ 10⁻⁶ cells) | T½ (min) |
|---|---|---|---|
| 4-octyl itaconate | Human | 401 | 4 |
| | Mouse | 351 | 4 |
| Example 1 | Human | 11 | 116 |
| | Mouse | 13 | 151 |
| Example 2 | Human | 8 | 170 |
| | Mouse | 35 | 35 |
| Example 4 | Human | 10 | 147 |
| | Mouse | NT* | NT |
| Example 5 | Human | 15 | 89 |
| | Mouse | 25 | 68 |
| Example 7 | Human | 20 | 66 |
| | Mouse | 14 | 118 |
| Example 8 | Mouse | 26 | 65 |
| | Human | 11 | 124 |
| Example 9 | Human | 15 | 91 |
| | Mouse | 44 | 38 |
| Example 11 | Mouse | 24 | 71 |
| | Human | 6 | 217 |
| Example 12 | Mouse | 42 | 45 |
| | Human | 46 | 37 |
| Example 13 | Human | <3 | >460 |
| | Mouse | 42 | 37 |
| Example 17 | Mouse | 9 | 212 |
| | Human | 11 | 157 |
| Example 18 | Mouse | 50 | 38 |
| | Human | 8 | 218 |
| Example 19 | Mouse | 23 | 81 |
| | Human | 4 | 447 |
| Example 20 | Human | 4 | 447 |
| 4-octyl itaconate | Human | 401 | 4 |
| | Mouse | 351 | 4 |
| | Mouse | 13 | 140 |
| Example 21 | Human | 31 | 55 |
| | Mouse | 71 | 27 |
| Example 22 | Human | <3 | >460 |
| | Mouse | 4 | 532 |
| Example 23 | Human | <3 | >460 |
| | Mouse | 12 | 126 |
| Example 24 | Human | <3 | >460 |
| | Mouse | 11 | 145 |
| Example 25 | Human | 4 | 339 |
| | Mouse | 9 | 177 |
| Example 26 | Human | 238 | 6 |
| | Mouse | >460 | <3 |
| Example 27 | Human | <3 | >460 |
| | Mouse | <3 | >460 |
| Example 29 | Human | 11 | 143 |
| | Mouse | 20 | 79 |
| Example 30 | Human | 41 | 37 |
| | Mouse | 104 | 15 |
| Example 31 | Mouse | 38 | 42 |
| | Human | 11 | 140 |
| Example 32 | Human | 17 | 92 |
| | Mouse | 21 | 76 |
| Example 34 | Human | 26 | 52 |
| | Mouse | 48 | 26 |
| Example 35 | Human | 14 | 86 |
| | Mouse | 73 | 30 |
| Example 36 | Human | 12 | 112 |
| | Mouse | >460 | <3 |
| Example 37 | Human | <3 | >460 |
| | Mouse | 34 | 48 |
| Example 38 | Human | 7 | 183 |
| | Mouse | 13 | 163 |
| 4-octyl itaconate | Human | 401 | 4 |
| | Mouse | 351 | 4 |
| Example 39 | Human | 18 | 65 |
| | Mouse | 45 | 48 |
| Example 40 | Human | 12 | 101 |
| | Mouse | 25 | 89 |
| Example 41 | Human | <3 | >460 |
| | Mouse | 8 | 292 |
| Example 42 | Human | 4 | 448 |
| | Mouse | <3 | >460 |
| Example 44 | Human | <3 | >460 |
| | Mouse | 9 | 140 |
| Example 45 | Human | <3 | >460 |
| | Mouse | 8 | 156 |
| Example 46 | Human | <3 | >460 |
| | Mouse | <3 | >460 |
| Example 47 | Human | <3 | >460 |
| | Mouse | <3 | >460 |
| Example 49 | Human | 91 | 15 |
| | Mouse | 68 | 19 |
| Example 50 | Human | <3 | >460 |
| | Mouse | 4 | 420 |
| Example 51 | Human | <3 | >460 |
| | Mouse | <3 | >460 |
| Example 52 | Human | <3 | >460 |
| | Mouse | 5 | 307 |
| Example 55 | Human | NT | NT |
| | Mouse | 29 | 55 |
| Example 57 | Human | <3 | >460 |
| | Mouse | 10 | 151 |
| Example 58 | Human | 5 | 372 |
| | Mouse | 5 | 327 |
| Example 59 | Human | 11 | 138 |
| | Mouse | <3 | >460 |
| Example 61 | Human | 6 | 215 |
| 4-octyl itaconate | Human | 401 | 4 |
| | Mouse | 351 | 4 |
| | Mouse | 21 | 103 |
| Example 62 | Mouse | 4 | 434 |
| | Human | <3 | >460 |

*NT means not tested in this assay

The results indicate that the compounds of the invention, at least those of Table 3, are expected to have acceptable or improved metabolic stabilities, as shown by their intrinsic clearance (Cliₙₜ) and half-life (T_{1/2}) values, in this assay. All compounds in Table 3 were more stable, i.e., they exhibited lower intrinsic clearance (Cliₙₜ) and/or longer half-life (T_{1/2}) values compared with 4-octyl itaconate in at least human or mouse species. Preferred compounds exhibited lower intrinsic clearance (Cliₙₜ) and longer half-life (T_{1/2}) values compared with 4-octyl itaconate in both human and mouse species.

### REFERENCES

The following publication cited in this specification are herein incorporated by reference in their entirety.
Ackermann et al. Proc. Soc. Exp. Bio. Med. 1949, 72(1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLoS One 2015, 10, e0120254.
BrQck J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Daly R. et al. medRxiv 2019, 19001594; doi: https://doi.org/10.1101/19001594.
Daniels B. P. et al. Immunity 2019, 50(1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020 doi:10.1111/imcb.12316.
Hanke T. et al. Pharmacol. Therapeut. 2016, 157, 163-187.
Hunt T. et al. Consortium of Multiple Sclerosis Centers 2015 Annual Meeting, 27-30 May 2015, Indianapolis, IN, USA: Poster DX37.
Kornberg M. D. et al. Science 2018, 360, 449-453.
Kulkarni R. A. et al. Nat. Chem. Biol. 2019 10.1038/s41589-018-0217-y.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10(1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Yi F. et al. Hepatology 2020, 873, 172989.
Yu X.-H. et al. Immunol. Cell Biol. 2019, 97, 134-141.
Zhang S. et al. Bioorg. Med. Chem. 2012, 20, 6073-6079.
Zhang D. et al. Int. Immunopharmacol. 2019, 77, 105924.
Zhao C. et al. Microb. Pathogen. 2019, 133, 103541.
Zhao G. et al. Biochem. Biophys. Res. Commun. 2014, 448, 303-307.

### Miscellaneous

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1: A compound of formula (I): 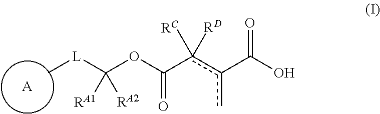
wherein, 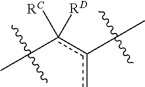
in the compound of formula (I) represents: 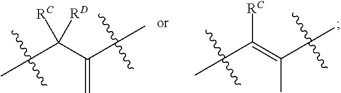
L is a bond, C₁₋₂ alkylene or C≡C;
R^{A1} is H, C₁₋₄ alkyl, C₁₋₄haloalkyl or C≡CH; and
R^{A2} is H, C₁₋₄ alkyl or C₁₋₄ haloalkyl; or
R^{A1} and R^{A2} join to form a C₃₋₆ cycloalkyl ring: 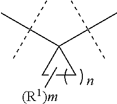
wherein:
R¹ is independently fluoro, methyl, cyano, OCH₃ or CF₃, or two R¹ groups which are attached to the same carbon atom join to form a C₃₋₄ cycloalkyl ring;
m is 0, 1 or 2;
n is 1, 2, 3 or 4; or
R^{A1} and R^{A2} join to form a 4-6 membered heterocyclic ring wherein the 4-6 membered heterocyclic ring is optionally substituted by one or more groups selected from oxo, fluoro or methyl;
A is phenyl, naphthyl or 5-6 membered heteroaryl wherein A is optionally substituted by one or more R², wherein A is other than pyrazolyl;
R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, halo, SF₅, SC₁₋₄ alkyl, SC₁₋₄ haloalkyl, —C≡C—C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring;
wherein when A is phenyl, L is a bond or C≡C, and R² is R^{2a}, R^{2b}, R^{2c}, R^{2d} and R^{2e}, the compound of formula (I) is: 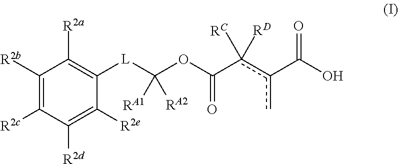
wherein:
R^{2a} is H or halo;
R^{2b} is H or halo;
R^{2c} is C₁₋₄ alkyl, C₁₋₄ alkoxy, C₁₋₄ haloalkyl, C₁₋₄ haloalkoxy, cyano, Cl, Br, SF₅, SC₁₋₄ haloalkyl, —C≡C≡C₁₋₂haloalkyl and phenyl, wherein the phenyl is optionally substituted by halo, or C₁₋₂ haloalkyl, and wherein the C₁₋₄ haloalkyl is optionally substituted by phenyl;
R^{2d} is H or fluoro; and
R^{2e} is H;
wherein when R^{2a}, R^{2d} and R^{2e} are H, R^{2b} is Cl and R^{2c} is CF₃, R^{A2} is other than H;
wherein when A is naphthyl, R² is independently selected from the group consisting of C₁₋₄ alkyl, C₁₋₄ alkoxy, C₁₋₄ haloalkyl, C₁₋₄ haloalkoxy, hydroxy, CO₂H, cyano, methanesulfonyl, chloro, bromo, SF₅, SC₁₋₄ alkyl, SC₁₋₄ haloalkyl, —C—C≡C₁₋₂haloalkyl, and phenyl, wherein the phenyl is optionally substituted by halo or C₁₋₂ haloalkyl and wherein the C₁₋₄ haloalkyl is optionally substituted by phenyl; or, two R² groups are attached to adjacent carbon atoms and are joined to form a C₃₋₈ cycloalkyl or 4-7 membered heterocyclic ring; and
wherein when A is thienyl, R² is C₁₋₄ haloalkyl;
R^{C} and R^{D} are independently selected from the group consisting of H, C₁₋₂ alkyl, hydroxy, methoxy and fluoro or, taken together, R^{C} and R^{D} combine to form a C₃₋₄ cycloalkyl ring;
or a pharmaceutically acceptable salt and/or solvate thereof.

2-12. (canceled)

13: The compound or a pharmaceutically acceptable salt and/or solvate thereof according to claim 1 wherein R^{A1} and R^{A2} join to form a C₃₋₆ cycloalkyl ring: 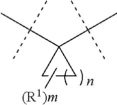
wherein:
R¹ is independently fluoro, methyl or cyano, or two R¹ groups which are attached to the same carbon atom join to form a C₃₋₄ cycloalkyl ring;
m is 0, 1 or 2;
n is 1,2,3 or 4.

14-28. (canceled)

29: The compound or a pharmaceutically acceptable salt and/or solvate thereof according to claim 1 wherein R^{A1} and R^{A2} join to form a 4-6 membered heterocyclic ring.

30-31. (canceled)

32: The compound or a pharmaceutically acceptable salt and/or solvate thereof according to claim 1, wherein A is phenyl optionally substituted by one or more R².

33. (canceled)

34: The compound or a pharmaceutically acceptable salt and/or solvate thereof according to claim 1, wherein A is 5-6 membered heteroaryl optionally substituted by one or more R².

35-41. (canceled)

42: The compound or a pharmaceutically acceptable salt and/or solvate thereof according to any claim 1 wherein one R² is C₁₋₄ haloalkyl, C₁₋₄ haloalkoxy, halo or C₁₋₄ haloalkyl.

43-59. (canceled)

60: The compound or a pharmaceutically acceptable salt and/or solvate thereof according to claim 1 wherein A is phenyl, L is a bond or C≡C, and R² is R^{2a}, R^{2b}, R^{2c}, R^{2d} and R^{2e}, such that the compound of formula (I) is: 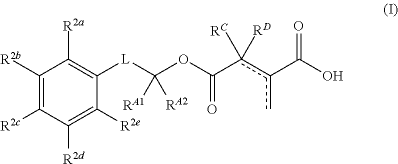
wherein:
R^{2a} is H or halo;
R^{2b} is H or halo;
R^{2c} is C₁₋₄ alkyl, C₁₋₄ alkoxy, C₁₋₄ haloalkyl, C₁₋₄ haloalkoxy, cyano, Cl, Br, SF₅, SC₁₋₄ haloalkyl, —C≡C—C₁₋₂haloalkyl and phenyl, wherein the phenyl is optionally substituted by halo, or C₁₋₂ haloalkyl, and wherein the C₁₋₄ haloalkyl is optionally substituted by phenyl;
R^{2d} is H or fluoro; and
R^{2e} is H;
wherein when R^{2a}, R^{2d} and R^{2e} are H, R^{2b} is Cl and R^{2c} is CF₃, R^{A2} is other than H.

61: The compound or a pharmaceutically acceptable salt and/or solvate thereof according to claim 60 wherein R^{2a} is H.

62-63. (canceled)

64. The compound or a pharmaceutically acceptable salt and/or solvate thereof according to claim 60 wherein R^{2b} is halo.

65-66. (canceled)

67: The compound or a pharmaceutically acceptable salt and/or solvate thereof according to a m claim 60 wherein R^{2c} is C₁₋₄ haloalkyl, C₁₋₄ haloalkoxy, Cl, Br and SC₁₋₄ haloalkyl.

68-77. (canceled)

78: The compound or a pharmaceutically acceptable salt and/or solvate thereof according to claim 1 wherein L is a bond.

79-80. (canceled)

81: The compound or a pharmaceutically acceptable salt and/or solvate thereof according to claim 1, wherein R^{C} is H and R^{D} is H.

82-110. (canceled)

111: The compound or a pharmaceutically acceptable salt and/or solvate thereof according to claim 1 wherein 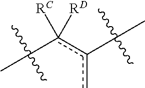
in the compound of formula (I) represents: 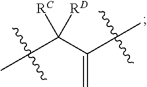
wherein R^{C} and R^{D} are as defined in claim 1.

112. (canceled)

113: The compound or a pharmaceutically acceptable salt and/or solvate thereof according to claim 1 which is selected from the group consisting of:
2-methylene-4-oxo-4-(1-(4-(trifluoromethyl)phenyl)cyclobutoxy)butanoic acid;
2-methylene-4-oxo-4-((2-(4-(trifluoromethyl)phenyl)propan-2-yl)oxy)butanoic acid;
(R)-2-methylene-4-oxo-4-(1-(4-(trifluoromethyl)phenyl)ethoxy)butanoic acid;
(S)-2-methylene-4-oxo-4-(1-(4-(trifluoromethyl)phenyl)ethoxy)butanoic acid;
4-(1-(4-bromophenyl)cyclobutoxy)-2-methylene-4-oxobutanoic acid;
4-(1-(2-chloro-4-(trifluoromethyl)phenyl)cyclobutoxy)-2-methylene-4-oxobutanoic acid;
2-methylene-4-oxo-4-((1-(4-(trifluoromethyl)phenyl)cyclopentyl)oxy)butanoic acid;
4-(1-(4-chlorophenyl)cyclobutoxy)-2-methylene-4-oxobutanoic acid;
2-methylene-4-oxo-4-(1-(4-((trifluoromethyl)thio)phenyl)cyclobutoxy)butanoic acid;
2-methylene-4-oxo-4-(1-(4-(trifluoromethyl)phenyl)propoxy)butanoic acid;
2-methylene-4-oxo-4-(1-(4-(trifluoromethyl)phenyl)cyclopropoxy)butanoic acid;
4-(1-(3-chloro-4-(trifluoromethyl)phenyl)cyclobutoxy)-2-methylene-4-oxobutanoic acid;
2-methylene-4-oxo-4-((4-(trifluoromethyl)benzyl)oxy)butanoic acid;
4-(1-(3-chloro-4-methylphenyl)cyclobutoxy)-2-methylene-4-oxobutanoic acid;
2-methylene-4-oxo-4-(1-(4-(trifluoromethoxy)phenyl)cyclobutoxy)butanoic acid;
4-(1-(4′-fluoro-[1,1′-biphenyl]-4-yl)cyclobutoxy)-2-methylene-4-oxobutanoic acid;
4-(1-(3-fluoro-4-(trifluoromethyl)phenyl)cyclobutoxy)-2-methylene-4-oxobutanoic acid;
2-methylene-4-oxo-4-(1-(4-(pentafluoro-I⁶-sulfaneyl)phenyl)cyclobutoxy)butanoic acid;
4-(1-(4-(1,1-difluoroethyl)phenyl)cyclobutoxy)-2-methylene-4-oxobutanoic acid;
4-(1-(4-(difluoromethyl)phenyl)cyclobutoxy)-2-methylene-4-oxobutanoic acid;
(E)-2-methyl-4-oxo-4-(1-(4-(trifluoromethyl)phenyl)cyclobutoxy)but-2-enoic acid;
2-methylene-4-oxo-4-(1-(6-(trifluoromethyl)pyridin-3-yl)cyclobutoxy)butanoic acid;
4-((3-(4-bromophenyl)oxetan-3-yl)oxy)-2-methylene-4-oxobutanoic acid;
2-methylene-4-oxo-4-((3-(4-(trifluoromethyl)phenyl)oxetan-3-yl)oxy)butanoic acid;
4-(1-(4-cyanophenyl)cyclobutoxy)-2-methylene-4-oxobutanoic acid;
2-methylene-4-oxo-4-(1-(4′-(trifluoromethyl)-[1,1′-biphenyl]-4-yl)cyclobutoxy)butanoic acid;
2-methylene-4-oxo-4-(1-(5-(trifluoromethyl)pyridin-2-yl)cyclobutoxy)butanoic acid;
4-(1-(4-(difluoro(phenyl)methyl)phenyl)cyclobutoxy)-2-methylene-4-oxobutanoic acid;
4-(3,3-difluoro-1-(4-(trifluoromethyl)phenyl)cyclobutoxy)-2-methylene-4-oxobutanoic acid;
3,3-dimethyl-2-methylene-4-oxo-4-(1-(4-(trifluoromethyl)phenyl)cyclobutoxy) butanoic acid;
2-methylene-4-oxo-4-(1-(4-(perfluoroethyl)phenyl)cyclobutoxy)butanoic acid;
2-methylene-4-oxo-4-(1-(5-(trifluoromethyl)thiophen-2-yl)cyclobutoxy)butanoic acid;
(S)-2-methylene-4-oxo-4-(2,2,2-trifluoro-1-(4-(trifluoromethyl)phenyl)ethoxy)butanoic acid;
(R)-2-methylene-4-oxo-4-(2,2,2-trifluoro-1-(4-(trifluoromethyl)phenyl)ethoxy)butanoic acid;
2-methylene-4-oxo-4-((2-(4-(trifluoromethyl)phenyl)spiro[3.3]heptan-2-yl)oxy)butanoic acid;
2-methylene-4-oxo-4-(1-(4-(3,3,3-trifluoroprop-1-yn-1-yl)phenyl)cyclobutoxy)butanoic acid;
2-methylene-4-oxo-4-((1-(4-(trifluoromethyl)phenyl)prop-2-yn-1-yl)oxy)butanoic acid (Enantiomer 1);
2-methylene-4-oxo-4-((1-(4-(trifluoromethyl)phenyl)prop-2-yn-1-yl)oxy)butanoic acid (Enantiomer 2);
2-(1-((1-(4-(trifluoromethyl)phenyl)cyclobutoxy)carbonyl)cyclopropyl)acrylic acid;
2-methylene-4-oxo-4-((3-(4-(trifluoromethyl)phenyl)thietan-3-yl)oxy)butanoic acid;
4-((1s,3s)-3-cyano-1-(4-(trifluoromethyl)phenyl)cyclobutoxy)-2-methylene-4-oxobutanoic acid;
4-(1-(3,5-difluoro-4-(trifluoromethyl)phenyl)cyclobutoxy)-2-methylene-4-oxobutanoic acid;
2-methylene-4-oxo-4-(1-((4-(trifluoromethyl)phenyl)ethynyl) cyclobutoxy)butanoic acid;
2-methylene-4-oxo-4-((5-(4-(trifluoromethyl)phenyl)spiro[2.3]hexan-5-yl)oxy)butanoic acid;
2-methylene-4-oxo-4-((4-(4-(trifluoromethyl)phenyl)tetrahydro-2H-pyran-4-yl)oxy)butanoic acid;
4-((1,1-dioxido-3-(4-(trifluoromethyl)phenyl)thietan-3-yl)oxy)-2-methylene-4-oxobutanoic acid;
4-((1-methyl-3-(4-(trifluoromethyl)phenyl)azetidin-3-yl)oxy)-2-methylene-4-oxobutanoic acid;
2-methylene-4-oxo-4-(3-(5-(trifluoromethyl)pyridin-2-yl)oxetan-3-yloxy)butanoic acid;
4-(3-methyl-1-(4-(trifluoromethyl)phenyl)cyclobutoxy)-2-methylene-4-oxobutanoic acid;
4-(3-methyl-1-(4-(trifluoromethyl)phenyl)cyclobutoxy)-2-methylene-4-oxobutanoic acid;
2-methylene-4-oxo-4-((5-(trifluoromethyl)pyridin-2-yl)methoxy)butanoic acid;
4-(1-(5-chloropyridin-2-yl)cyclobutoxy)-2-methylene-4-oxobutanoic acid;
4-((3-fluoro-4-(trifluoromethyl)benzyl)oxy)-2-methylene-4-oxobutanoic acid;
4-(1-(5-bromopyridin-2-yl)cyclobutoxy)-2-methylene-4-oxobutanoic acid;
4-(1-(6-bromonaphthalen-2-yl)cyclobutoxy)-2-methylene-4-oxobutanoic acid;
2-methylene-4-oxo-4-(3-(trifluoromethyl)-1-(4-(trifluoromethyl)phenyl)cyclobutoxy)butanoic acid;
2-methylene-4-oxo-4-((2-(5-(trifluoromethyl)pyridin-2-yl)propan-2-yl)oxy)butanoic acid;
4-(3-methoxy-1-(4-(trifluoromethyl)phenyl)cyclobutoxy)-2-methylene-4-oxobutanoic acid;
3,3-dimethyl-2-methylene-4-oxo-4-(1-(5-(trifluoromethyl)pyridin-2-yl)cyclobutoxy)butanoic acid;
2-methylene-4-oxo-4-(1-(5-(trifluoromethyl)pyrimidin-2-yl)cyclobutoxy)butanoic acid;
2-methylene-4-oxo-4-(1-(5-(trifluoromethyl)pyridin-2-yl)cyclopropoxy)butanoic acid;2-methylene-4-oxo-4-(1-(6-(trifluoromethyl)pyridazin-3-yl)cyclobutoxy)butanoic acid; and
2-methylene-4-oxo-4-(1-(5-(trifluoromethyl)pyrazin-2-yl)cyclobutoxy)butanoic acid;
or a pharmaceutically acceptable salt and/or solvate of any one thereof.

114: A pharmaceutical composition comprising a compound or a pharmaceutically acceptable salt and/or solvate thereof according to claim 1.

115-117. (canceled)

118: A method of treating or preventing an inflammatory disease or a disease associated with an undesirable immune response, which comprises administering a compound or a pharmaceutically acceptable salt and/or solvate thereof according to claim 1.

119-122. (canceled)

123. The method according to claim 118, wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease autoimmune paraneoplastic retinopathy, transplantation rejection (including antibody-mediated and T cell-mediated forms), multiple sclerosis, transverse myelitis, ischaemia-reperfusion injury, AGE-induced genome damage, an inflammatory bowel disease, primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff s brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders, and renal inflammatory disorders.

124-130. (canceled)

131: The compound or a pharmaceutically acceptable salt and/or solvate thereof according to claim 1, for use in combination with a further therapeutic agent; selected from the group consisting of a corticosteroid (glucocorticoid), retinoid, anthralin, vitamin D analogue, calcineurin inhibitors, phototherapy or photochemotherapy or other form of ultraviolet light irradiation therapy, ciclosporine, a thiopurine, methotrexate, an anti-TNFα agents, phosphodiesterase-4 (PDE4) inhibition anti-IL-17 agent, anti-IL12/IL-23 agent, anti-IL-23 agent, JAK (Janus Kinase) inhibitor, plasma exchange, intravenous immune globulin (IVIG), cyclophosphamide, anti-CD20 B cell depleting agent, anthracycline analogue, cladribine, sphingosine 1-phosphate receptor modulator or sphingosine analogue, interferon beta preparation (including interferon beta 1b/1a), glatiramer, anti-CD3 therapy, anti-CD52 targeting agent, leflunomide, teriflunomide, gold compound, laquinimod, potassium channel blocker, mycophenolic acid, mycophenolate mofetil, purine analogue, mTOR (mechanistic target of rapamycin) pathway inhibitor, anti-thymocyte globulin (ATG), IL-2 receptor (CD25) inhibitor, anti-IL-6 receptor or anti-IL-6 agent, Bruton's tyrosine kinase (BTK) inhibitor, tyrosine kinase inhibitor, ursodeoxycholic acid, hydroxychloroquine, chloroquine, B cell activating factor (BAFF, also known as BLyS, B lymphocyte stimulator) inhibitor, other B cell targeted therapy including a fusion protein targeting both APRIL (A PRoliferation-Inducing Ligand) and BLyS, PI3K inhibitor including pan-inhibitor or one targeting the p110δ and/or p110γ containing isoforms, an interferon α receptor inhibitor, T cell co-stimulation blocker, thalidomide and its derivatives, dapsone, clofazimine, a leukotriene antagonist, theophylline, anti-IgE therapy, an anti-IL-5 agent, a long-acting muscarinic agent, a PDE4 inhibitor, riluzole, a free radical scavenger, a proteasome inhibitor, a complement cascade inhibitor including one directed against C5, immunoadsor, antithymocyte globulin, 5-aminosalicylates and their derivatives, an anti-integrin agent including one targeting α4β1 and/or α4β7 integrins, an anti-CD11-α agent, a non-steroidal anti-inflammatory drug (NSAID) including a salicylate, a propionic acid, an acetic acid, an oxicam, a fenamate, a selective or relatively selective COX-2 inhibitor, colchicine, an IL-4 receptor inhibitor, topical/contact immunotherapy, anti-IL-1 receptor therapy, IL-1β inhibitor, IL-1 neutralising therapy, chlorambucil, a specific antibiotic with immunomodulatory properties and/or ability to modulate NRF2, anti-androgenic therapy, pentoxifylline, ursodeoxycholic acid, obeticholic acid, fibrate, a cystic fibrosis transmembrane conductance regulator (CFTR) modulator, a VEGF (vascular endothelial growth factor), pirfenidone and mizoribine.

132: A compound which is selected from the group consisting of:
(a) a compound of formula (II): 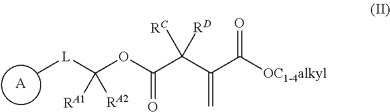
or a salt thereof, wherein the C₁₋₄alkyl group is optionally substituted by halo, and A, L, R^{A1}, R^{A2}, R^{C} and R^{D} are defined in claim 1;
(b) a compound of formula (III): 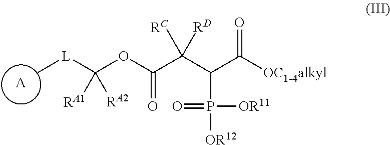
or a salt thereof, wherein the C₁₋₄alkyl group is optionally substituted by halo, A, L, R^{A1}, R^{A2}, R^{C} and R^{D} are defined in claim 1, and R¹¹ and R¹² independently represent C₁₋₄ alkyl optionally substituted with halo;
(c) a compound of formula (V): 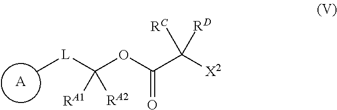
or a salt thereof, wherein A, L, R^{A1}, R^{A2}, R^{C} and R^{D} are defined in claim 1, and X² is a leaving group;
(d) a compound of formula (X); 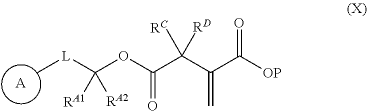
or a salt thereof, wherein A, L, R^{A1}, R^{A2}, R^{C} and R^{D} are defined in claim 1, and P is a carboxylic acid protecting group; and
(e) a compound of formula (XII): 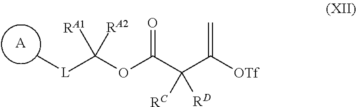
or a salt thereof, wherein A, L, R^{A1}, R^{A2}, R^{C} and R^{D} are as defined in claim 1.

133-139. (canceled)

140: A process for preparing a compound of formula (I) or a salt thereof, which comprises:
(a) hydrolysing a compound of formula (II): 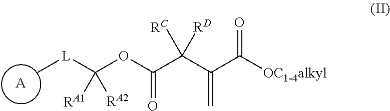
or a salt thereof,
wherein the C₁₋₄alkyl group is optionally substituted by halo, and A, L, R^{A1}, R^{A2}, R^{C} and R^{D} are defined in claim 1;
(b) deprotecting a compound of formula (X): 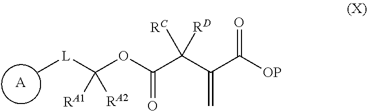
or a salt thereof;
wherein A, L, R^{A1}, R^{A2}, R^{C} and R^{D} are defined in claim 1, and P is a carboxylic acid protecting group; or
(c) reacting a compound of formula (XII): 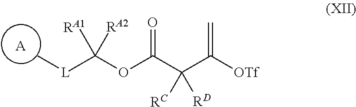
or a salt thereof;
with carbon monoxide under transition metal-catalysed conditions;
wherein A, L, R^{A1}, R^{A2}, R^{C} and R^{D} are as defined in claim 1.

141-143. (canceled)
